(19) United States
(12) Patent Application Publication
(10) Pub. No.: US 2024/0150315 A1
(43) Pub. Date: May 9, 2024

(54) BETA-2 ADRENORECEPTOR MODULATORS AND METHODS OF USING SAME

(71) Applicants: University of Maryland, Baltimore, Baltimore, MD (US); Thomas Jefferson University, Philadelphia, PA (US)

(72) Inventors: Alexander D. MacKerell, Baltimore, MD (US); Deepak Deshpande, North Wales, PA (US); Christopffer Lind, Headington (GB); Sushrut D. Shah, Bensalem, PA (US)

(21) Appl. No.: 18/482,497

(22) Filed: Oct. 6, 2023

Related U.S. Application Data
(60) Provisional application No. 63/378,777, filed on Oct. 7, 2022.

(51) Int. Cl. C07D 401/06; A61P 11/06; C07D 403/04; C07D 403/06

(52) U.S. Cl. CPC C07D 401/06; A61P 11/06; C07D 403/04; C07D 403/06

(57) ABSTRACT

The disclosure relates to allosteric modulators of β₂-adrenoceptor, pharmaceutical compositions thereof, and use thereof for the treatment of disease.

### CROSS-REFERENCE TO RELATED APPLICATIONS

This application claims priority to and benefit of U.S. Provisional Patent Application No. 63/378,777, filed Oct. 7, 2022, which is incorporated by reference herein in its entirety.

### STATEMENT AS TO FEDERALLY SPONSORED RESEARCH

This invention was made with government support under Grant Numbers GM131710 and AI135082 awarded by the National Institutes of Health. The government has certain rights in the invention.

### FIELD

The disclosure relates generally to compounds that are allosteric modulators of β₂-adrenoceptor, and methods of using such compounds as treatments for disease.

### BACKGROUND

Activation of β₂-adrenoceptors (β₂ARs) causes airway smooth muscle (ASM) relaxation and bronchodilation, and β₂AR agonists (β-agonists) are front-line treatments for asthma and other obstructive lung diseases. However, the therapeutic efficacy of β-agonists is limited by agonist-induced β₂AR desensitization and activation of non-canonical β₂AR signaling involving β-arrestin shown to promote asthma pathophysiology. There is a need for further improvement of current treatment regimens.

### SUMMARY

In one embodiment, the disclosure provides a compound of formula (I), or a pharmaceutically acceptable salt, solvate, hydrate, cocrystal, or prodrug thereof:

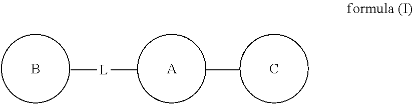

wherein in formula (I), A is an optionally substituted 6-membered heteroaryl or optionally substituted 6-membered heterocyclyl, provided that the optionally substituted 6-membered heteroaryl or optionally substituted 6-membered heterocyclyl comprises two or more nitrogen atoms; B is an optionally substituted monocyclic aryl or optionally substituted monocyclic or bicyclic heteroaryl; C is an optionally substituted heteroaryl or optionally substituted cycloalkyl; L is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —CR^{a}═N—NR^{a}—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and R^{a} is each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl.

In some embodiments, A is selected from optionally substituted pyrimidine, optionally substituted pyridazine, optionally substituted pyrazine, and optionally substituted piperazine. In some embodiments, A is selected from:

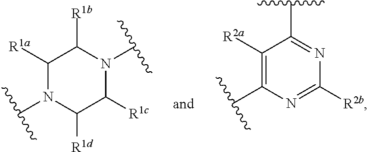

wherein R^{1a}, R^{1b}, R^{1c}, R^{1d}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)N(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl; R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In some embodiments, B is optionally substituted aryl, optionally substituted pyridyl, or optionally substituted quinoxaline. In some embodiments, C is optionally substituted 3- to 7-membered cycloalkyl, optionally substituted pyrrole, optionally substituted imidazole, optionally substituted pyrazole, or optionally substituted triazole.

In some embodiments, the disclosure provides a compound of formula (1), or a pharmaceutically acceptable salt, solvate, hydrate, cocrystal, or prodrug thereof:

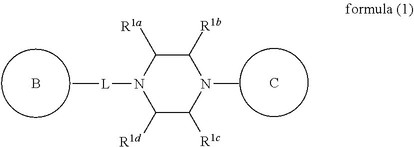

wherein in formula (1), B is an optionally substituted monocyclic heteroaryl; and C is an optionally substituted 3- to 7-membered cycloalkyl.

In some embodiments, the disclosure provides a compound of formula (2), or a pharmaceutically acceptable salt, solvate, hydrate, cocrystal, or prodrug thereof:

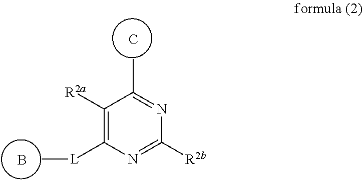

wherein in formula (2), B is an optionally substituted monocyclic aryl; and C is an optionally heteroaryl.

In some embodiments, B is selected from:

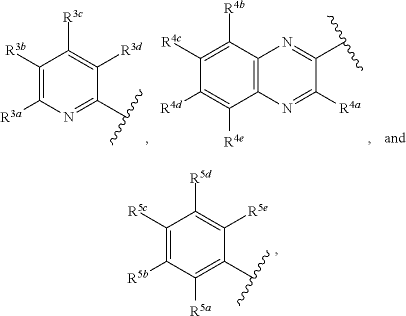

wherein R^{3a}, R^{3b}, R^{3c}, R^{3d}, R^{4a}, R^{4b}, R^{4c}, R^{4d}, R^{4e}, R^{5a}, R^{5b}, R^{5c}, R^{5d}, and R^{5e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl; R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In some embodiments, the disclosure provides a compound of formula (10), or a pharmaceutically acceptable salt, solvate, hydrate, cocrystal, or prodrug thereof:

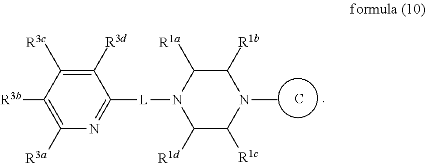

In some embodiments, the disclosure provides a compound of formula (11), or a pharmaceutically acceptable salt, solvate, hydrate, cocrystal, or prodrug thereof:

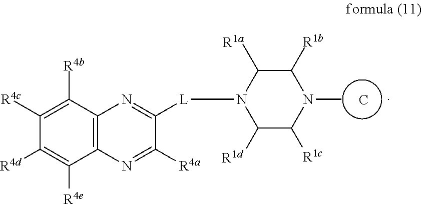

In some embodiments, R^{1a}, R^{1b}, and R^{1d} are each H. In some embodiments, R^{1c} is substituted C₁₋₆ alkyl, optionally substituted ethyl, optionally —(CH₂)₂—OH. In some embodiments, R^{3a}, R^{3b}, R^{3c}, and R^{3d} are each H. In some embodiments, R^{4b}, R^{4c}, R^{4d}, and R^{4e} are each H. In some embodiments, R^{4a} is C₁₋₆ alkyl, optionally —CH₃.

In some embodiments, C is selected from:

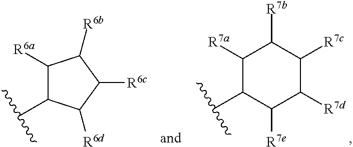

wherein R^{6a}, R^{6b}, R^{6C}, R^{6d}, R^{7a}, R^{7b}, R^{7c}, R^{7d}, and R^{7e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl; R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In some embodiments, C is:

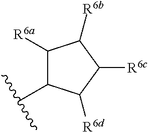

and
R^{6a}, R^{6b}, R^{6c}, and R^{6d} are each H. In other embodiments, C is:

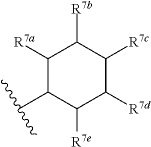

and
R^{7a}, R^{7b}, R^{7c}, R^{7d}, and R^{7e} are each H.

In some embodiments, L is —(CH₂)₁₋₆—, optionally L is —(CH₂)—.

In some embodiments, the disclosure provides a compound of formula (3), or a pharmaceutically acceptable salt, solvate, hydrate, cocrystal, or prodrug thereof:

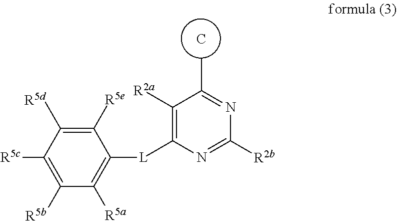

wherein in formula (3), B is an optionally substituted monocyclic aryl; and C is an optionally heteroaryl.

In some embodiments, R^{2a} and R^{2b} are each H. In some embodiments, R^{5b}, R^{5c}, R^{5d}, and R^{5e} are each H. In some embodiments, R^{5a} is —OR^{a}, optionally —OH. In some embodiments, C is:

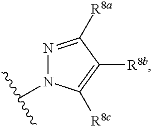

wherein R^{8a}, R^{8b}, and R^{8c} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R)S(O)R^{a}, —C(O)N(R^{a})S(OR^{a}, —S(O)OR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl; R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and t is 1 or 2.

In some embodiments, R^{8a} and R^{8c} are each H. In some embodiments, R^{8b} is C₁₋₆ alkyl, optionally —CH₃. In some embodiments, L is —CR^{a}═N—NR^{a}—, optionally L is —CH═N—NH—.

In some embodiments, the compound is an allosteric activator of the β₂-adrenoceptor. In other embodiments, the compound is an allosteric inhibitor of the β₂-adrenoceptor.

In some embodiments, the compound is not a compound of any one of formula 1001-1008:

| Compound # | Structure |
|---|---|
| 1001 | 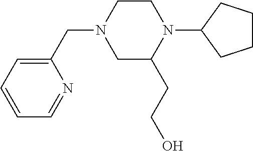 [SMILES: OCCC1CN(Cc2ccccn2)CCN1C1CCCC1] |
| 1002 | 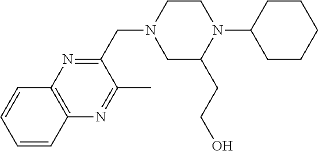 [SMILES: Cc1nc2ccccc2nc1CN1CCN(C2CCCCC2)C(CCO)C1] |
| 1003 | 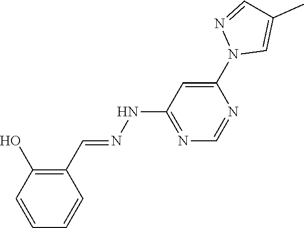 [SMILES: Cc1cnn(-c2cc(N/N=C/c3ccccc3O)ncn2)c1] |
| 1004 | 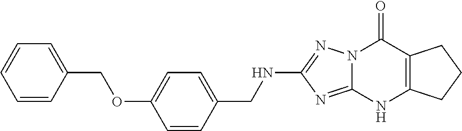 [SMILES: O=c1c2c([nH]c3nc(NCc4ccc(OCc5ccccc5)cc4)nn13)CCC2] |
| 1005 | 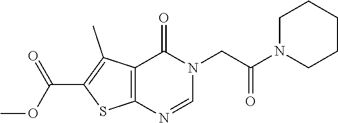 [SMILES: COC(=O)c1sc2ncn(CC(=O)N3CCCCC3)c(=O)c2c1C] |
| 1006 | 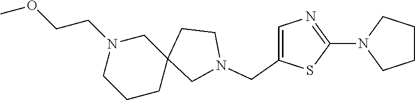 [SMILES: COCCN1CCCC2(CCN(Cc3cnc(N4CCCC4)s3)C2)C1] |
| 1007 | 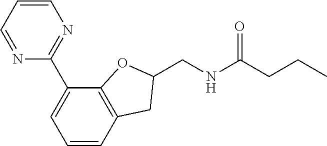 [SMILES: CCCC(=O)NCC1Cc2cccc(-c3ncccn3)c2O1] |
| 1008 | 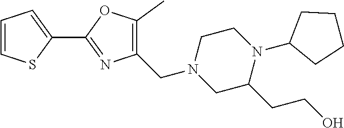 [SMILES: Cc1oc(-c2cccs2)nc1CN1CCN(C2CCCC2)C(CCO)C1] |

In one embodiment, the disclosure provides a pharmaceutical composition comprising a compound of formula (I), formula (1), formula (2), formula (10), formula (11), formula (3), or a pharmaceutically acceptable salt, solvate, hydrate, cocrystal, or prodrug thereof, and a physiologically compatible carrier medium.

In one embodiment, the disclosure provides a method of treating a disease or disorder alleviated by allosterically modulating β₂-adrenoceptor in a patient in need thereof, the method comprising administering to the patient a therapeutically effective amount of a compound of formula (I), formula (1), formula (2), formula (10), formula (11), formula (3), or a pharmaceutically acceptable salt, solvate, hydrate, cocrystal, or prodrug thereof.

In one embodiment, the disclosure provides a method of treating a disease or disorder alleviated by allosterically modulating β₂-adrenoceptor in a patient in need thereof, the method comprising administering to the patient a pharmaceutical composition comprising a compound of formula (I), formula (1), formula (2), formula (10), formula (11), formula (3), or a pharmaceutically acceptable salt, solvate, hydrate, cocrystal, or prodrug thereof.

In some embodiments, the method further comprises allosterically activating β₂-adrenoceptor. In some embodiments, the disease or disorder is an inflammatory disease. In some embodiments, the inflammatory disease is selected from the group consisting of chronic obstructive pulmonary disease (COPD), asthma, acute respiratory distress syndrome (ARDS), and acute lung injury (ALI), optionally wherein the inflammatory disease is asthma or COPD. In some embodiments, the method further comprises allosterically inhibiting β₂-adrenoceptor. In some embodiments, the disease or disorder is a cardiovascular disease. In some embodiments, the cardiovascular disease is selected from the group consisting of cardiac arrhythmia, cardiomyopathy, myocardial infarction, heart valve disease, and heart failure, optionally wherein the cardiovascular disease is heart failure.

In some embodiments, the method comprises administering to the patient a therapeutically effective amount of a compound of formula 1001, or a pharmaceutically acceptable salt, solvate, hydrate, cocrystal, or prodrug thereof:

| Compound # | Structure |
|---|---|
| 1001 | 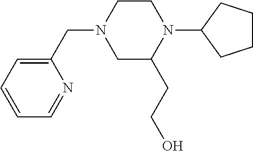 [SMILES: OCCC1CN(Cc2ccccn2)CCN1C1CCCC1] |

In some embodiments, the method comprises administering to the patient a therapeutically effective amount of a compound of formula 1002, or a pharmaceutically acceptable salt, solvate, hydrate, cocrystal, or prodrug thereof:

| Compound # | Structure |
|---|---|
| 1002 | 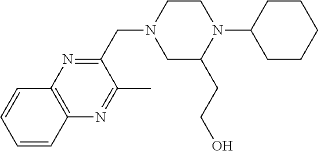 [SMILES: Cc1nc2ccccc2nc1CN1CCN(C2CCCCC2)C(CCO)C1] |

In some embodiments, the method comprises administering to the patient a therapeutically effective amount of a compound of formula 1003, or a pharmaceutically acceptable salt, solvate, hydrate, cocrystal, or prodrug thereof:

| Compound # | Structure |
|---|---|
| 1003 | 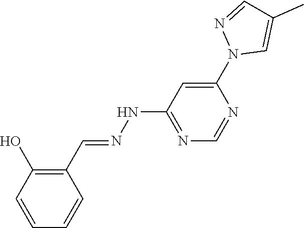 [SMILES: Cc1cnn(-c2cc(N/N=C/c3ccccc3O)ncn2)c1] |

In some embodiments, the method comprises administering the compound, or pharmaceutically acceptable salt, solvate, hydrate, cocrystal, or prodrug thereof in a dosage unit form. In some embodiments, the dosage unit form comprises a physiologically compatible carrier medium.

### BRIEF DESCRIPTION OF THE DRAWINGS

The foregoing summary, as well as the following detailed description of embodiments of the disclosure, are better understood when read in conjunction with the appended drawings and figures.

FIGS. 1A-1E show results from molecular dynamics (MD) simulations used to identify the β₂AR intermediate conformational states. FIG. 1A is a graph of Climber Energy (kcal/mol) (blue solid line) and RMSD with respect to inactive (red dashed line) and active (red dash-dotted line) conformations (PDBIDs: 5X7D and 3SN6, respectively) during the morphing from the active to inactive form of β₂AR. Black dots display the local energy minima along the morphing pathway selected for the two starting intermediate conformations. FIG. 1B shows the time evolution of the ionic lock distance from the MD simulations in the presence of BI-167107. Green vertical dashed lines represent the ionic lock alpha carbon distance measured from the inactive (˜11 Å) and active (˜19.5 Å) receptor. FIG. 1C shows the time evolution of the ionic lock distance from the MD simulations in the presence of carazolol. Green vertical dashed lines represent the ionic lock alpha carbon distance measured from the inactive (˜11 Å) and active (˜19.5 Å) receptor. FIG. 1D shows the density distributions of the ionic lock distance from the MD simulations in the presence of BI-167107. FIG. 1E shows the density distributions of the ionic lock distance from the MD simulations in the presence of carazolol. The simulations in FIG. 1B and FIG. 1C were initiated from the Step74 conformation.

FIGS. 2A-2H illustrate the SILCS simulations used to identify allosteric binding sites on the β₂AR. FIG. 2A shows the SILCS FragMaps as mesh representations overlaid on the structure of the Step74 conformation used to initiate the SILCS simulations [SILCS FragMaps: apolar (green, −0.9), hydrogen bond donor (blue, −0.6), hydrogen bond acceptor (red, −0.6), negative (orange, −1.2), and positive (cyan, −1.2), where the energy indicates the isocontour cutoff in kcal/mol; Step74 structure: blue, cartoon]. FIG. 2B shows the SILCS Hotspots throughout the entire protein (VDW spheres, scaled by 0.7, colored by LGFE score from red to blue for the most favorable to least favorable ranked Hotspots). FIG. 2C shows 3 HotSpots selected that define the identified allosteric site (red transparent oval) located below the orthosteric site (cyan transparent rectangle) between helices 3, 5, 6 and 7 (TM helix numbers shown except 4, which is on the back of the image). FIG. 2D is the Step74 structure in the region of the allosteric binding site showing residues defining the site (sticks, atom colored) and 3 selected Hotspots in the presence of the Solvent Accessible Surface (white). FIG. 2E is the Step74 structure in the region of the allosteric binding site showing residues defining the site (sticks, atom colored) and 3 selected Hotspots in the presence of the SILCS Exclusion Map (tan). Compounds shown in FIGS. 2F-2H are the SILCS docked orientations of positive or negative allosteric modulators 1003 (FIG. 2F), 1001 (FIG. 2G), and 1002 (FIG. 2G) (CPK, atom colored) overlaid on the side chains of the residues defining the allosteric binding site (stick, atom colored) and the pharmacophores used to select the respective compounds (VDW spheres, hydrophobic (cyan), hydrogen bond acceptor (red), and hydrogen bond donor (blue)). The pharmacophores in FIG. 2G and FIG. 2H are identical. Residue numbers in FIG. 2F are based on the Ballesteros nomenclature (PDB IDs shown in FIG. 3).

FIG. 3 shows the list of residues defining the allosteric binding site and helix number.

FIG. 4 shows the chemical structures of the allosteric modulators.

FIGS. 5A-5F illustrate the effect of β₂AR modulators on agonist-induced cAMP accumulation. Cells were plated into a 24-well plate and stimulated with vehicle or β₂AR modulators in combination with isoproterenol (ISO) for 10 minutes and cAMP accumulation was measured by ELISA as described in Methods. Isoproterenol (ISO)/β₂AR modulators dose-response curve was generated using a non-linear (4-parameter) logistic curve from multiple experiments (n=6). All statistical significances were determined using two-way ANOVA with Bonferroni post hoc analysis. FIG. 5A is a graph of experimental data illustrating the stimulation of HEK293 cells expressing β₂AR with different concentrations of allosteric modulators (AMs) or ISO. FIG. 5B is a graph of experimental data illustrating the stimulation of HEK293 cells expressing β₂AR with different concentrations of AMs and 100 nM ISO. FIG. 5C is a graph of experimental data illustrating the co-stimulation of HEK293 cells with the top ranking PAM and NAMs (100 nM) combined with different concentrations of ISO. n=5-8, * p<0.05 (compound vs. ISO). FIG. 5D is a graph of experimental data illustrating the co-stimulation of human ASM cells with the top ranking PAM and NAMs (100 nM) combined with different concentrations of ISO. n=5-8, * p<0.05 (compound vs. ISO). FIG. 5E is a graph of experimental data illustrating the co-stimulation of HEK293 cells expressing human β₁AR with AMs (100 nM) combined with varying concentrations of ISO. n=6. FIG. 5F is a graph of experimental data illustrating the stimulation of ASM cells with different concentrations of prostaglandin E₂ with vehicle or 100 nM AMs. n=5. Error bars represent±SEM.

FIGS. 6A-6B illustrate the effect of β₂AR modulators on isoproterenol-induced cAMP accumulation. Cells were plated into a 24 well plate and co-stimulated with vehicle or β₂AR modulators (100 nM) and isoproterenol (ISO) for 10 minutes and cAMP accumulation was measured by ELISA as described in Example 1. Isoproterenol dose-response curve was generated using non-linear (4-parameter) logistic curve from multiple experiments (n=6). All statistical significances were determined using two-way ANOVA with Bonferroni post hoc analysis. FIG. 6A is a graph of experimental data illustrating the co-stimulation of HEK293 cells overexpressing β₂AR with vehicle and ISO, or β₂AR modulators (100 nM) and ISO. n=5-8, * p<0.05 (compound vs. ISO). FIG. 6B is a graph of experimental data illustrating the co-stimulation of human ASM cells with either vehicle and ISO, or β₂AR modulators (100 nM) and ISO. n=5-8, * p<0.05 (compound vs. ISO). All compounds that modulated isoproterenol-induced cAMP generation in HEK293 cells overexpressing β₂AR (FIG. 6A) and human ASM cells (FIG. 6B). Error bars represent ±SEM.

FIGS. 7A-7B show the effect of β₂AR modulators on cAMP accumulation. FIG. 7A shows the calculated EC₅₀ and Eₘₐₓ for cAMP accumulation in HEK293 cells stimulated with different concentrations of ISO in the presence of vehicle or 100 nM AMs. FIG. 7B shows the calculated EC₅₀ and Eₘₐₓ for cAMP accumulation in human ASM cells stimulated with different concentrations of ISO in the presence of vehicle or 100 nM AMs.

FIGS. 8A-8C illustrate the effect of β₂AR modulators on ISO-induced β-arrestin recruitment and phosphorylation of ERK1/2. FIG. 8A is a graph of experimental data illustrating cells expressing BRET reporters (Rluc-β₂AR and GFP-β-arrestin2) treated with increasing concentrations of ISO and vehicle or 100 nM AMs; change in fluorescence was measured using a plate reader. ISO treatment enhanced recruitment of β-arrestin to the receptor and co-treatment of cells with AMs did not increase ISO-induced β-arrestin recruitment to the β₂AR (n=5). Error bars represent±SEM. Graphpad Prism was used to generate non-linear regression curves (4-PL) and two-way ANOVA with Bonferroni post hoc was used for statistical analysis. FIG. 8B shows ISO-induced activation of β-arrestin-ERK1/2 assessed by western blotting. FIG. 8C is a graph of experimental data illustrating the band intensities from the western blots of FIG. 8B normalized to β-actin. Data are expressed as fold change from vehicle-treated cells. AMs (100 nM) did not further modulate 100 nM ISO-induced phosphorylation of ERK in HEK293 cells. n=4 * p<0.05 ISO vs. AMs+ISO using Student's t-test. Error bars represent±SEM (FIG. 8B and FIG. 8C).

FIGS. 9A-9B illustrate the effect of β₂AR modulators on activation of ISO-induced β-arrestin signaling in human ASM cells. FIG. 9A shows ISO-induced β-arrestin-mediated phosphorylation of ERK1/2 in human ASM cells assessed by western blotting. FIG. 9B is a graph of experimental data illustrating the band intensities from the western blots of FIG. 9A normalized to β-actin. The data are represented as fold change from basal. AMs (100 nM) did not modulate 100 nM ISO-induced phosphorylation of ERK1/2. n=4 * p<0.05 ISO vs. AMs+ISO using Student's t-test. Error bars represent±SEM.

FIGS. 10A-10D illustrate the effect of AMs on the binding affinity of ISO to the β₂AR. Cell membranes expressing the β₂AR were incubated with 3 nM [³H]DHA and with increasing concentrations of ISO in the presence or absence of AMs. Non-specific binding was assessed with 10 mM alprenolol. Binding competition curves were generated using the function log(inhibitor) vs. response (three parameters) of the non-linear regression curve fitting in GraphPad Prism and are represented as a percentage of the maximal [³H]DHA specific binding. FIG. 10A is a graph of experimental data showing the effect of AM compounds 1001, 1003, and 1002 (100 nM) on the binding affinity of ISO to β₂AR. FIG. 10B is a graph of experimental data showing the effect of compound 1001 (1 μM, 3 μM, and 10 μM) on the binding affinity of ISO to β₂AR. FIG. 10C is a graph of experimental data showing the effect of compound 1003 (1 μM, 3 μM, and 10 μM) on the binding affinity of ISO to β₂AR. FIG. 10D is a graph of experimental data showing the effect of compound 1002 (1 μM, 3 IM, and 10 μM) on the binding affinity of ISO to β₂AR. n=4. Error bars represent ±SEM

FIGS. 11A-11E illustrate the experimental studies used to validate the identification of the allosteric modulator binding site on the β₂AR. FIG. 11A shows the amino acids that form the putative binding pocket for the AMs on β₂AR identified by SILCS computational modeling.

FIG. 11B is a graph of experimental data illustrating the stimulation of HEK293 cells expressing Wild Type (WT) and mutant β₂AR with 1 μM ISO (cAMP accumulation was measured upon stimulation). FIG. 11C is a graph of experimental data illustrating the co-stimulation of cells expressing WT and Y219 mutant β₂AR with either vehicle and ISO (1 μM), or β₂AR modulators (100 nM) and ISO (1 μM). cAMP accumulation was measured after 10 min. of stimulation (n=3-4, * p<0.05 Compound+ISO vs. Veh+ISO, #p<0.05 WT vs. β₂AR mutant). FIG. 11D is a graph of experimental data illustrating the co-stimulation of cells expressing WT and R131 mutant β₂AR with either vehicle and ISO (1 μM), or β₂AR modulators (100 nM) and ISO (1 μM). cAMP accumulation was measured after 10 min. of stimulation (n=3-4, * p<0.05 Compound+ISO vs. Veh+ISO, #p<0.05 WT vs. β₂AR mutant). FIG. 11E is a graph of experimental data illustrating the co-stimulation of cells expressing WT and F282 mutant β₂AR with either vehicle and ISO (1 μM), or β₂AR modulators (100 nM) and ISO (1 IM). cAMP accumulation was measured after 10 min. of stimulation (n=3-4, * p<0.05 Compound+ISO vs. Veh+ISO, #p<0.05 WT vs. β₂AR mutant). Student's t-test was used to determine statistical significances. Error bars represent f SEM.

FIGS. 12A-12D show cAMP generation by different concentrations of ISO in HEK293 cells expressing mutant β₂AR. FIG. 12A is a graph of experimental data illustrating the stimulation of HEK293 cells expressing Wild Type (WT) and mutant β₂AR with different concentrations of ISO (cAMP accumulation was measured upon stimulation). FIG. 12B is a graph of experimental data illustrating the co-stimulation of cells expressing WT and Y219A mutant β₂AR with either vehicle and different concentrations of ISO, or β₂AR AMs (100 nM) and different concentrations of ISO. cAMP accumulation was measured after 10 min. of stimulation. FIG. 12C is a graph of experimental data illustrating the co-stimulation of cells expressing WT and R131A mutant β₂AR with either vehicle and different concentrations of ISO, or β₂AR AMs (100 nM) and different concentrations of ISO. cAMP accumulation was measured after 10 min. of stimulation. FIG. 12D is a graph of experimental data illustrating the co-stimulation of cells expressing WT and F282A mutant β₂AR with either vehicle and different concentrations of ISO, or β₂AR AMs (100 nM) and different concentrations of ISO. cAMP accumulation was measured after 10 min. of stimulation. Regression curves were generated using four-parameter non-linear regression curve fit in Graphpad Prism and two-way ANOVA was employed to assess statistical differences. n=6, * p<0.05 (WT vs. mutants). Error bars represent±SEM.

FIGS. 13A-13B show the effect of allosteric modulators on cell surface expression of β₂AR. FIG. 13A is a graph of experimental data illustrating the cell surface expression of β₂AR (measured by ELISA) in HEK293 cells overexpressing wild-type (WT) or mutant β₂AR with an N-terminal 3×-HA tag that were briefly fixed with 10% formalin-buffered saline (n=6). FIG. 13B is a graph of experimental data illustrating the cell surface expression of β₂AR (measured by cell surface ELISA) in HEK293 cells overexpressing wild-type (WT) or mutant β₂AR that were stimulated with ISO (100 nM) with vehicle or compound 1001, 1003, or 1002 (100 nM) for 15 min (n=6). No significance between WT vs β₂AR and Veh+ISO vs 37+Iso. HA, hemagglutinin.

FIGS. 14A-14C show the effect of allosteric modulators on β-agonist-mediated inhibition of contraction of human ASM cells and augmentation of bronchodilation in human and murine lung slices. FIG. 14A is a graph of experimental data collected from images of collagen gels containing human ASM cells that were pre-treated with veh or AMs (100 nM) with different concentrations of ISO for 10 min and stimulated with histamine (10 μM). Images of collagen gels were taken at 10-minute intervals over 30 minutes and normalized to the basal area for each measure. Data are representative of n=6-10 independent donors. * p<0.05 His vs. ISO+His, #p<0.05 Veh+ISO vs. Compound+ISO. His, histamine. FIG. 14B is a graph of experimental data illustrating human lung slices treated with methacholine (1 μM) for 10 min and then treated with vehicle or β₂AR modulators (100 nM) for 10 min, followed by treatment with an increasing concentration of ISO. FIG. 14C is a graph of experimental data illustrating murine lung slices treated with methacholine (1 μM) for 10 min and then treated with vehicle or β₂AR modulators (100 nM) for 10 min, followed by treatment with an increasing concentration of ISO. Change in lumen area of airways was used to determine the magnitude of bronchodilation. n=8, * p<0.05 Veh+ISO vs. Compounds+ISO. Non-linear curve fitting (Inhibitor vs three-parameter for FIG. 14C and agonist vs four-parameter logistic curve fit for FIG. 14B and FIG. 14C) was generated using Graphpad Prism software. All statistical differences were analyzed using two-way ANOVA with Bonferroni post hoc analysis. Error bars represent f SEM.

FIG. 15 shows the effect of allosteric modulators on bronchodilation in murine precision-cut lung slices. Murine lung slices were contracted with methacholine (1 μM) for 10 min. Pre-contracted lung slices were treated with vehicle or 100 nM β₂AR modulators for 10 min followed by treatment with increasing concentrations of isoproterenol. Change in lumen area of airways was used to determine magnitude of bronchodilation. All the lead modulators were tested in lung slices. n=8, * p<0.05 Veh+ISO vs. Compounds+ISO. Non-linear curve fitting (agonist vs four-parameter logistic curve fit) was generated using Graphpad Prism software. All statistical differences were analyzed using two-way ANOVA with Bonferroni post hoc analysis. Error bars represent±SEM.

FIGS. 16A-16B show the effect of β₂AR modulators on ISO-mediated relaxation of precision cut lung slices. FIG. 16A shows the calculated EC₅₀ and Eₘₐₓ of ISO for bronchodilation of human precision cut lung slices in the presence of vehicle or AMs (100 nM).

FIG. 16B shows the calculated EC₅₀ and Eₘₐₓ of ISO for bronchodilation of murine precision cut lung slices in the presence of vehicle or AMs (100 nM).

FIGS. 17A-17C illustrate predicted bound orientations of compounds 1003, 1001, and 1002. FIG. 17A shows compound 1003 overlaid on the SILCS FragMaps. FIG. 17B shows compound 1001 overlaid on the SILCS FragMaps. FIG. 17C shows compound 1002 overlaid on the SILCS FragMaps. Selected residues in the allosteric binding site are included in FIG. 17C. Compounds in atom-colored CPK and residue sidechain non-hydrogen atoms in atom-colored licorice representations. Shown are apolar (green), hydrogen bond donor (blue), hydrogen bond acceptor (red) and negative (orange) SILCS FragMaps at −0.9 kcal/mol free energy contours and the positive (cyan) FragMaps at −0.6 kcal/mol contour. Apolar fragmaps indicated by green arrows overlap with the terminal rings of the compounds indicating the contribution of the rings to binding. In the central region of the binding site positive (indicated by cyan arrow) and both hydrogen bond donor (blue arrows) and acceptor (red arrow) FragMaps are present indicating how both the positive piperazine in 1001 and 1002 and hydrazine and pyrimidine moieties in 1003 have favorable interactions in the central region of the binding site. The FragMaps are identical in all three panels and arrows indicating important FragMaps are presented in FIG. 17A and FIG. 17B for clarity.

FIG. 18 is a graph of experimental data illustrating bronchodilation in response to inhaled isoproterenol (ISO) or ISO+compound 1001 was assessed in mice after methacholine (MCh)-induced bronchoconstriction by flexiVent (n=12).

FIGS. 19A-19E illustrate experimental data demonstrating that s-agonist augments HDM-induced features of asthma in mice. FIG. 19A illustrates an image of hematoxylin and eosin (H&E) staining demonstrating that intranasal treatment of mice with formoterol enhanced HDM-induced airway inflammation as assessed by H&E staining (top row), while formoterol enhanced HDM-induced mucus production (bottom row, Periodic acid-Schiff (PAS) staining). FIGS. 19B and 19C are graphs of experimental data demonstrating the quantification of histology images was carried out by a deconvolution method using Image J. FIG. 19D is a table illustrating total and differential (flow cytometric evaluation) BAL cell count. FIG. 19E is a graph of experimental data AHR assessed by flexiVent (C). Secretion of multiple cytokines was also significantly enhanced by formoterol treatment (data not shown). n=3-4 * p<0.05 PBS vs veh+HDM, #p<0.05 veh+HDM vs F100+HDM.

FIG. 20 is a scheme illustrating a non-limiting example of an allergen challenge and ligand treatment regimen. Animals are treated with allosteric modulators (AMs) and β-agonists in a prophylactic and treatment model.

### DETAILED DESCRIPTION

Obstructive lung diseases such as asthma and chronic obstructive pulmonary disease are characterized by increased bronchoconstriction and difficulty in breathing. Augmented airway smooth muscle (ASM) contraction contributes significantly to the narrowing of the airway lumen and various G protein-coupled receptors (GPCRs) expressed on the cell surface of ASM regulate contraction or relaxation of ASM, and thus airway patency. Activation of β₂-adrenoceptors (β₂AR) on ASM by endogenous (epinephrine and norepinephrine) and exogenous agonists promotes ASM relaxation and bronchodilation. Thus, synthetic agonists of the β₂AR (β-agonists) have been the principal therapeutic means of relief of acute bronchoconstriction for decades. However, chronic β-agonist use has multiple deleterious effects, including worsening asthma control associated with increased airway hyperesponsiveness and a loss of bronchoprotective effect as well as increased mucus production and indices of airway inflammation. Of particular note, the findings from the Salmeterol Multi-center Asthma Research Trial (SMART) study raised mortality concerns that ultimately led to a black box warning for long-acting β-agonists and more stringent guidelines for their clinical use. These studies underscore the need for drug development strategies for β₂AR ligands/modulators to effectively promote beneficial therapeutic effects while minimizing the deleterious effects of β-agonists.

β-agonists bind to β₂AR at the orthosteric site of the receptor and promote the interaction of the β₂AR with multiple signaling protein assemblies, including those of Gs-protein, G protein receptor kinases, and β-arrestins. These signaling proteins are capable of promoting multiple downstream signaling pathways resulting in ASM relaxation, negative feedback regulation (desensitization, receptor internalization), gene expression, and regulation of cell proliferation. Numerous cell, tissue, and in vivo studies have attributed the therapeutic effects of β-agonists in asthma to canonical Gs signaling by the β₂AR, while the regulatory effects of β-arrestins constrain such therapeutic efficacy by promoting ASM β₂AR desensitization, and reducing canonical Gs-cAMP-PKA signaling and the pro-relaxant effect of β-agonists on ASM. More recent studies have implicated β-arrestins in β-agonist-induced pro-inflammatory and pathogenic effects in murine models of asthma.

Development of allosteric modulators of the β₂AR, particularly those capable of augmenting and biasing β₂AR downstream signaling towards (therapeutic) signaling capabilities may overcome the limitations of those β-agonists currently used to manage asthma. Allosteric modulators bind to a receptor at sites distinct from those bound by orthosteric ligands, to either augment (Positive Ailosteric Modulator (PAM)) or reduce (Negative Allosteric Modulator (NAM)) the signaling transduced by the orthosteric ligand. Allosteric modulators (AMs) of GPCRs have potential advantages over orthosteric ligands as therapeutic drugs. In the absence of an orthosteric ligand, they lack activity and thus are able to maintain activity dependence of physiological GPCR signaling. Moreover, allosteric modulators offer the potential of greater specificity of targeting, given the greater sequence diversity of allosteric modulator binding sites relative to those typically engaged by orthosteric ligands. To date, a few allosteric modulators of β₂AR have been identified, with their effects on modulating signaling and arrestin recruitment characterized in HEK293 cells expressing recombinant β₂AR. Experimental screening using DNA-encoded libraries identified a β₂AR NAM and a PAM (Cmpd 6), with both compounds having similar (negative and positive) modulation of β-agonist-induced cAMP generation and arrestin recruitment. A weak β₂AR NAM was identified using in silico docking, and chemical optimization resulted in a balanced NAM for both Gs activation and arrestin recruitment. Recent studies report PAM Cmpd 6 acts as a positive modulator of the beta blocker carvedilol, through enhanced binding affinity of carvedilol to β₂AR and enhanced β-arrestin binding. Cmpd 6 can also increase the activity of carvedilol (but not of β₁AR agonists) at the β₁AR and β₁AR-arrestin-mediated signaling to confer cardioprotection.

In one aspect, the disclosure describes an approach to rationally identify additional allosteric binding sites and small molecules capable of modulating signaling by β₂AR using the SILCS (site identification by ligand competitive saturation) technology, targeting a conformation intermediate to the active and inactive states of the β₂AR modeled through the program Climber. SILCS is a co-solvent molecular simulation approach that maps the functional group affinity pattern of a protein, including the protein interior, taking into account protein flexibility as well as protein and functional group desolvation contributions. The SILCS-Hotspots method allows for docking a collection of fragments on the entire protein to identify putative binding sites suitable for drug-like molecules. The SILCS approach has previously been used to identify allosteric sites on the proteins heme oxygenase and β-Glucosidase A. In addition, the SILCS-Pharmacophore and Monte Carlo (SILCS-MC) methods are of utility for identifying drug-like molecules that bind to the targeted sites, an approach previously used to identify novel agonists of the β₂AR. In embodiments, the disclosure provides the use of SILCS toolbox and molecular dynamics (MD) simulations to identify a previously unidentified allosteric site on β₂AR along with putative AMs that interact with the identified site, which were then subjected to robust signaling and functional characterization.

### I. Definitions

Unless defined otherwise, all technical and scientific terms used herein have the same meaning as is commonly understood by one of skill in the art to which this disclosure belongs. All patents and publications referred to herein are incorporated by reference in their entireties.

“Alkyl” refers to a straight or branched hydrocarbon chain radical consisting solely of carbon and hydrogen atoms, containing no unsaturation, having from one to ten carbon atoms (e.g., (C₁₋₁₀)alkyl or C₁₋₁₀ alkyl). Whenever it appears herein, a numerical range such as “1 to 10” refers to each integer in the given range, e.g., “1 to 10 carbon atoms” means that the alkyl group may consist of 1 carbon atom, 2 carbon atoms, 3 carbon atoms, etc., up to and including 10 carbon atoms, although the definition is also intended to cover the occurrence of the term “alkyl” where no numerical range is specifically designated. Typical alkyl groups include, but are in no way limited to, methyl, ethyl, propyl, isopropyl, n-butyl, isobutyl, sec-butyl isobutyl, tertiary butyl, pentyl, isopentyl, neopentyl, hexyl, septyl, octyl, nonyl and decyl. The alkyl moiety may be attached to the rest of the molecule by a single bond, such as for example, methyl (Me), ethyl (Et), n-propyl (nPr), 1-methylethyl (isopropyl), n-butyl, n-pentyl, 1,1-dimethylethyl (t-butyl) and 3-methylhexyl. Unless stated otherwise specifically in the specification, an alkyl group is optionally substituted by one or more of substituents which are independently heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a}), —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂ where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkylaryl” refers to an -(alkyl)aryl radical where aryl and alkyl are as disclosed herein and which are optionally substituted by one or more of the substituents described as suitable substituents for aryl and alkyl respectively.

“Alkylheteroaryl” refers to an -(alkyl)heteroaryl radical where heteroaryl and alkyl are as disclosed herein and which are optionally substituted by one or more of the substituents described as suitable substituents for aryl and alkyl respectively.

“Alkylheterocycloalkyl” refers to an -(alkyl) heterocyclic radical where alkyl and heterocycloalkyl are as disclosed herein and which are optionally substituted by one or more of the substituents described as suitable substituents for heterocycloalkyl and alkyl respectively.

An “alkene” moiety refers to a group consisting of at least two carbon atoms and at least one carbon-carbon double bond, and an “alkyne” moiety refers to a group consisting of at least two carbon atoms and at least one carbon-carbon triple bond. The alkyl moiety, whether saturated or unsaturated, may be branched, straight chain, or cyclic.

“Alkenyl” refers to a straight or branched hydrocarbon chain radical group consisting solely of carbon and hydrogen atoms, containing at least one double bond, and having from two to ten carbon atoms (i.e., (C₂₋₁₀)alkenyl or C₂₋₁₀ alkenyl). Whenever it appears herein, a numerical range such as “2 to 10” refers to each integer in the given range—e.g., “2 to 10 carbon atoms” means that the alkenyl group may consist of 2 carbon atoms, 3 carbon atoms, etc., up to and including 10 carbon atoms. The alkenyl moiety may be attached to the rest of the molecule by a single bond, such as for example, ethenyl (i.e., vinyl), prop-1-enyl (i.e., allyl), but-1-enyl, pent-1-enyl and penta-1,4-dienyl. Unless stated otherwise specifically in the specification, an alkenyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)R^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkenyl-cycloalkyl” refers to an -(alkenyl)cycloalkyl radical where alkenyl and cycloalkyl are as disclosed herein and which are optionally substituted by one or more of the substituents described as suitable substituents for alkenyl and cycloalkyl respectively.

“Alkynyl” refers to a straight or branched hydrocarbon chain radical group consisting solely of carbon and hydrogen atoms, containing at least one triple bond, having from two to ten carbon atoms (i.e., (C₂₋₁₀)alkynyl or C₂₋₁₀ alkynyl). Whenever it appears herein, a numerical range such as “2 to 10” refers to each integer in the given range—e.g., “2 to 10 carbon atoms” means that the alkynyl group may consist of 2 carbon atoms, 3 carbon atoms, etc., up to and including 10 carbon atoms. The alkynyl may be attached to the rest of the molecule by a single bond, for example, ethynyl, propynyl, butynyl, pentynyl and hexynyl. Unless stated otherwise specifically in the specification, an alkynyl group is optionally substituted by one or more substituents which independently are: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R)C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)NR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Alkynyl-cycloalkyl” refers to an -(alkynyl)cycloalkyl radical where alkynyl and cycloalkyl are as disclosed herein and which are optionally substituted by one or more of the substituents described as suitable substituents for alkynyl and cycloalkyl respectively.

“Acylsulfonamide” refers to the group —C(═O)NR^{a}—S(═O)₂R^{a}, where each R^{a} is independently hydrogen, alkyl, fluoroalkyl, carbocyclyl, carbocyclylalkyl, aryl, aralkyl, heterocycloalkyl, heterocycloalkylalkyl, heteroaryl, or heteroarylalkyl.

“Carboxaldehyde” refers to a —(C═O)H radical.

“Carbonyl” refers to the group —C(═O)—. Carbonyl groups may be substituted with the following exemplary substituents: alkyl, heteroalkyl, alkenyl, alkynyl, cycloalkyl, heterocycloalkyl, hydroxamate, acylsulfonamido, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —NR^{a}—OR^{a}—, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)NR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Carboxyl” refers to a —(C═O)OH radical.

“Cyano” refers to a —CN radical.

“Cycloalkyl” refers to a monocyclic or polycyclic radical that contains only carbon and hydrogen, and may be saturated, or partially unsaturated. Cycloalkyl groups include groups having from 3 to 10 ring atoms (i.e., (C₃₋₁₀)cycloalkyl or C₃₋₁₀ cycloalkyl). Whenever it appears herein, a numerical range such as “3 to 10” refers to each integer in the given range—e.g., “3 to 10 carbon atoms” means that the cycloalkyl group may consist of 3 carbon atoms, etc., up to and including 10 carbon atoms. Illustrative examples of cycloalkyl groups include, but are not limited to the following moieties: cyclopropyl, cyclobutyl, cyclopentyl, cyclopentenyl, cyclohexyl, cyclohexenyl, cycloheptyl, cyclooctyl, cyclononyl, cyclodecyl, norbornyl, and the like. Unless stated otherwise specifically in the specification, a cycloalkyl group is optionally substituted by one or more substituents which independently are: alkyl, heteroalkyl, alkenyl, alkynyl, cycloalkyl, acylsulfonamido, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —S(O)R^{a}— (where t is 1 or 2), —OC(O)—R^{a}, —N(R)₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)NR^{a} (where t is 1 or 2), —S(O)R^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Cycloalkyl-alkenyl” refers to a -(cycloalkyl)alkenyl radical where cycloalkyl and alkenyl are as disclosed herein and which are optionally substituted by one or more of the substituents described as suitable substituents for cycloalkyl and alkenyl, respectively.

“Cycloalkyl-heterocycloalkyl” refers to a -(cycloalkyl)heterocycloalkyl radical where cycloalkyl and heterocycloalkyl are as disclosed herein and which are optionally substituted by one or more of the substituents described as suitable substituents for cycloalkyl and heterocycloalkyl, respectively.

“Cycloalkyl-heteroaryl” refers to a -(cycloalkyl)heteroaryl radical where cycloalkyl and heteroaryl are as disclosed herein and which are optionally substituted by one or more of the substituents described as suitable substituents for cycloalkyl and heteroaryl, respectively.

The term “alkoxy” refers to the group —O-alkyl, including from 1 to 8 carbon atoms of a straight, branched, cyclic configuration and combinations thereof attached to the parent structure through an oxygen. Examples include, but are not limited to, methoxy, ethoxy, propoxy, isopropoxy, cyclopropyloxy and cyclohexyloxy. “Lower alkoxy” refers to alkoxy groups containing one to six carbons.

The term “substituted alkoxy” refers to alkoxy wherein the alkyl constituent is substituted (i.e., —O-(substituted alkyl)). Unless stated otherwise specifically in the specification, the alkyl moiety of an alkoxy group is optionally substituted by one or more substituents which independently are: alkyl, heteroalkyl, alkenyl, acylsulfonamido, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)NR^{a}— (where t is 1 or 2), —OC(O)—R^{a}, —N(R^{a}), —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)NR^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “alkoxycarbonyl” refers to a group of the formula (alkoxy)(C═O)— attached through the carbonyl carbon wherein the alkoxy group has the indicated number of carbon atoms. Thus a (C₁₋₆)alkoxycarbonyl group is an alkoxy group having from 1 to 6 carbon atoms attached through its oxygen to a carbonyl linker. “Lower alkoxycarbonyl” refers to an alkoxycarbonyl group wherein the alkoxy group is a lower alkoxy group.

The term “substituted alkoxycarbonyl” refers to the group (substituted alkyl)-O—C(O)— wherein the group is attached to the parent structure through the carbonyl functionality. Unless stated otherwise specifically in the specification, the alkyl moiety of an alkoxycarbonyl group is optionally substituted by one or more substituents which independently are: alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)NR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R³, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyl” refers to the groups (alkyl)-C(O)—, (aryl)-C(O)—, (heteroaryl)-C(O)—, (heteroalkyl)-C(O)— and (heterocycloalkyl)-C(O)—, wherein the group is attached to the parent structure through the carbonyl functionality. If the R radical is heteroaryl or heterocycloalkyl, the hetero ring or chain atoms contribute to the total number of chain or ring atoms. Unless stated otherwise specifically in the specification, the alkyl, aryl or heteroaryl moiety of the acyl group is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R)₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Acyloxy” refers to a R(C═O)O— radical wherein R is alkyl, aryl, heteroaryl, heteroalkyl or heterocycloalkyl, which are as described herein. If the R radical is heteroaryl or heterocycloalkyl, the hetero ring or chain atoms contribute to the total number of chain or ring atoms. Unless stated otherwise specifically in the specification, the R of an acyloxy group is optionally substituted by one or more substituents which independently are: alkyl, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SRI, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R)C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)NR^{a} (where t is 1 or 2), —S(O)ₜOR^{a} (where t is 1 or 2), —S(O)N(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl, unless stated otherwise specifically in the specification. When a —N(R^{a})₂ group has two R^{a} substituents other than hydrogen, they is combined with the nitrogen atom to form a 4-, 5-, 6- or 7-membered ring. For example, —N(R^{a})₂ is intended to include, but is not limited to, 1-pyrrolidinyl and 4-morpholinyl. Unless stated otherwise specifically in the specification, an amino group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)NR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

The term “substituted amino” also refers to N-oxides of the groups —NHR^{d}, and NR^{d}R^{d} each as described above. N-oxides is prepared by treatment of the corresponding amino group with, for example, hydrogen peroxide or m-chloroperoxybenzoic acid.

“Amide” or “amido” refers to a chemical moiety with formula —C(O)NR^{a}R^{b} or —NR^{a}C(O)R^{b}, where R^{a} and R^{b} are selected from the group consisting of hydrogen, alkyl, cycloalkyl, aryl, heteroaryl (bonded through a ring carbon) and heteroalicyclic (bonded through a ring carbon), each of which moiety may itself be optionally substituted. The R^{a} and R^{b} of —C(O)NR^{a}R^{b} amide may optionally be taken together with the nitrogen to which they are attached to form a 4-, 5-, 6- or 7-membered ring. Unless stated otherwise specifically in the specification, an amido group is optionally substituted independently by one or more of the substituents as described herein for alkyl, amino, cycloalkyl, aryl, heteroaryl, or heterocycloalkyl. An amide may be an amino acid or a peptide molecule attached to a compound disclosed herein, thereby forming a prodrug. The procedures and specific groups to make such amides are known to those of skill in the art and can readily be found in seminal sources such as Greene and Wuts, Protective Groups in Organic Synthesis, 3^{rd} Ed., John Wiley & Sons, New York, N.Y., 1999, which is incorporated herein by reference in its entirety.

“Aromatic” or “aryl” or “Ar” refers to an aromatic radical with six to ten ring atoms (e.g., C₆-C₁₀ aromatic or C₆-C₁₀ aryl) which has at least one ring having a conjugated pi electron system which is carbocyclic (e.g., phenyl, fluorenyl, and naphthyl). Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Whenever it appears herein, a numerical range such as “6 to 10” refers to each integer in the given range; e.g., “6 to 10 ring atoms” means that the aryl group may consist of 6 ring atoms, 7 ring atoms, etc., up to and including 10 ring atoms. The term includes monocyclic or fused-ring polycyclic (i.e., rings which share adjacent pairs of ring atoms) groups. Unless stated otherwise specifically in the specification, an aryl moiety is optionally substituted by one or more substituents which are independently alkyl, heteroalkyl, acylsulfonamido, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R³, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)R^{a} (where t is 1 or 2), —S(O)ₜR^{a} (where t is 1 or 2), —S(O)ₜOR^{a} (where t is 1 or 2), —S(O)N(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Aralkyl” or “arylalkyl” refers to an (aryl)alkyl-radical where aryl and alkyl are as disclosed herein and which are optionally substituted by one or more of the substituents described as suitable substituents for aryl and alkyl respectively.

“Ester” refers to a chemical radical of formula —COOR, where R is selected from the group consisting of alkyl, cycloalkyl, aryl, heteroaryl (bonded through a ring carbon) and heteroalicyclic (bonded through a ring carbon). The procedures and specific groups to make esters are known to those of skill in the art and can readily be found in seminal stheces such as Greene and Wuts, Protective Groups in Organic Synthesis, 3^{rd} Ed., John Wiley & Sons, New York, N.Y., 1999, which is incorporated herein by reference in its entirety. Unless stated otherwise specifically in the specification, an ester group is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, trifluoromethyl, trifluoromethoxy, nitro, trimethylsilanyl, —OR^{a}, —SR^{a}, —S(O)ₜR^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)OR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Fluoroalkyl” refers to an alkyl radical, as defined above, that is substituted by one or more fluoro radicals, as defined above, for example, trifluoromethyl, difluoromethyl, 2,2,2-trifluoroethyl, 1-fluoromethyl-2-fluoroethyl, and the like. The alkyl part of the fluoroalkyl radical may be optionally substituted as defined above for an alkyl group.

“Halo,” “halide,” or, alternatively, “halogen” is intended to mean fluoro, chloro, bromo or iodo. The terms “haloalkyl,” “haloalkenyl,” “haloalkynyl,” and “haloalkoxy” include alkyl, alkenyl, alkynyl and alkoxy structures that are substituted with one or more halo groups or with combinations thereof. For example, the terms “fluoroalkyl” and “fluoroalkoxy” include haloalkyl and haloalkoxy groups, respectively, in which the halo is fluorine.

“Heteroalkyl,” “heteroalkenyl,” and “heteroalkynyl” refer to optionally substituted alkyl, alkenyl and alkynyl radicals and which have one or more skeletal chain atoms selected from an atom other than carbon, e.g., oxygen, nitrogen, sulfur, phosphorus or combinations thereof. A numerical range may be given—e.g., C₁-C₄ heteroalkyl which refers to the chain length in total, which in this example is 4 atoms long. A heteroalkyl group may be substituted with one or more substituents which independently are: alkyl, heteroalkyl, alkenyl, alkynyl, acylsulfonamido, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)R^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)R^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heteroalkylaryl” refers to an -(heteroalkyl)aryl radical where heteroalkyl and aryl are as disclosed herein and which are optionally substituted by one or more of the substituents described as suitable substituents for heteroalkyl and aryl, respectively.

“Heteroalkylheteroaryl” refers to an -(heteroalkyl)heteroaryl radical where heteroalkyl and heteroaryl are as disclosed herein and which are optionally substituted by one or more of the substituents described as suitable substituents for heteroalkyl and heteroaryl, respectively.

“Heteroalkylheterocycloalkyl” refers to an -(heteroalkyl)heterocycloalkyl radical where heteroalkyl and heterocycloalkyl are as disclosed herein and which are optionally substituted by one or more of the substituents described as suitable substituents for heteroalkyl and heterocycloalkyl, respectively.

“Heteroalkylcycloalkyl” refers to an -(heteroalkyl)cycloalkyl radical where heteroalkyl and cycloalkyl are as disclosed herein and which are optionally substituted by one or more of the substituents described as suitable substituents for heteroalkyl and cycloalkyl, respectively.

“Heteroaryl” or “heteroaromatic” or “HetAr” refers to a 5- to 18-membered aromatic radical (e.g., C₅-C₁₃ heteroaryl) that includes one or more ring heteroatoms selected from nitrogen, oxygen and sulfur, and which may be a monocyclic, bicyclic, tricyclic or tetracyclic ring system. Whenever it appears herein, a numerical range such as “5 to 18” refers to each integer in the given range—e.g., “5 to 18 ring atoms” means that the heteroaryl group may consist of 5 ring atoms, 6 ring atoms, etc., up to and including 18 ring atoms. Bivalent radicals derived from univalent heteroaryl radicals whose names end in “-yl” by removal of one hydrogen atom from the atom with the free valence are named by adding “-idene” to the name of the corresponding univalent radical—e.g., a pyridyl group with two points of attachment is a pyridylidene. A N-containing “heteroaromatic” or “heteroaryl” moiety refers to an aromatic group in which at least one of the skeletal atoms of the ring is a nitrogen atom. The polycyclic heteroaryl group may be fused or non-fused. The heteroatom(s) in the heteroaryl radical are optionally oxidized. One or more nitrogen atoms, if present, are optionally quaternized. The heteroaryl may be attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzoxazolyl, benzopyranyl, benzopyranonyl, benzofuranyl, benzofuranonyl, benzofurazanyl, benzothiazolyl, benzothienyl(benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, isoxazol-3-one, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl). Unless stated otherwise specifically in the specification, a heteroaryl moiety is optionally substituted by one or more substituents which are independently: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)ₜR^{a}— (where t is 1 or 2), —OC(O)—R^{a}, —N(R)₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)ₜOR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

Substituted heteroaryl also includes ring systems substituted with one or more oxide (—O—) substituents, such as, for example, pyridinyl N-oxides.

“Heteroarylalkyl” refers to a moiety having an aryl moiety, as described herein, connected to an alkylene moiety, as described herein, wherein the connection to the remainder of the molecule is through the alkylene group.

“Heterocycloalkyl” or “heterocyclyl” refer to a stable 3- to 18-membered non-aromatic ring radical that comprises two to twelve carbon atoms and from one to six heteroatoms selected from nitrogen, oxygen and sulfur. Whenever it appears herein, a numerical range such as “3 to 18” refers to each integer in the given range—e.g., “3 to 18 ring atoms” means that the heterocycloalkyl group and/or heterocyclyl group may consist of 3 ring atoms, 4 ring atoms, etc., up to and including 18 ring atoms. Unless stated otherwise specifically in the specification, the heterocycloalkyl radical and/or heterocyclyl radical is a monocyclic, bicyclic, tricyclic or tetracyclic ring system, which may include fused or bridged ring systems. The heteroatoms in the heterocycloalkyl radical and/or heterocyclyl radical may be optionally oxidized. One or more nitrogen atoms, if present, are optionally quaternized. The heterocycloalkyl radical is partially or fully saturated. The heterocycloalkyl and/or heterocyclyl may be attached to the rest of the molecule through any atom of the ring(s). Examples of such heterocycloalkyl radicals and/or heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocycloalkyl moiety is optionally substituted by one or more substituents which independently are: alkyl, acylsulfonamido, heteroalkyl, alkenyl, alkynyl, cycloalkyl, heterocycloalkyl, hydroxamate, aryl, arylalkyl, heteroaryl, heteroarylalkyl, hydroxy, halo, cyano, nitro, oxo, thioxo, trimethylsilanyl, —OR^{a}, —SR^{a}, —S(O)R^{a}— (where t is 1 or 2), —OC(O)—R^{a}, —N(R^{a})₂, —C(O)R^{a}, —C(O)OR^{a}, —OC(O)N(R^{a})₂, —C(O)N(R^{a})₂, —N(R^{a})C(O)OR^{a}, —N(R^{a})C(O)R^{a}, —N(R^{a})C(O)N(R^{a})₂, N(R^{a})C(NR^{a})N(R^{a})₂, —N(R^{a})S(O)ₜR^{a} (where t is 1 or 2), —S(O)ₜR^{a} (where t is 1 or 2), —S(O)OR^{a} (where t is 1 or 2), —S(O)ₜN(R^{a})₂ (where t is 1 or 2), or PO(OR^{a})₂, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Heterocycloalkyl” and/or “heterocyclyl” also includes bicyclic ring systems wherein one non-aromatic ring, usually with 3 to 7 ring atoms, contains at least 2 carbon atoms in addition to 1-3 heteroatoms independently selected from oxygen, sulfur, and nitrogen, as well as combinations comprising at least one of the foregoing heteroatoms; and the other ring, usually with 3 to 7 ring atoms, optionally contains 1-3 heteroatoms independently selected from oxygen, sulfur, and nitrogen and is not aromatic.

“Hydroxamate” refers to the —C(O)NR^{a}OR^{a} moiety, where each R^{a} is independently hydrogen, alkyl, fluoroalkyl, carbocyclyl, carbocyclylalkyl, aryl, aralkyl, heterocycloalkyl, heterocycloalkylalkyl, heteroaryl or heteroarylalkyl.

“Nitro” refers to the —NO₂ radical.

“Oxa” refers to the —O— radical.

“Oxo” refers to the ═O radical.

“Isomers” are different compounds that have the same molecular formula. “Stereoisomers” are isomers that differ only in the way the atoms are arranged in space—i.e., having a different stereochemical configuration. “Enantiomers” are a pair of stereoisomers that are non-superimposable mirror images of each other. A 1:1 mixture of a pair of enantiomers is a “racemic” mixture. The term “(+)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer the stereochemistry at each chiral carbon is specified by either (R) or (S). Resolved compounds whose absolute configuration is unknown is designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that is defined, in terms of absolute stereochemistry, as (R) or (S). The present chemical entities, pharmaceutical compositions and methods are meant to include all such possible isomers, including racemic mixtures, optically pure forms and intermediate mixtures. Optically active (R)- and (S)-isomers is prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which may potentially have an (R)- or an (S)-isomeric configuration, is present as a racemic mixture, the enantiomeric purity is about 50% with respect to either the (R)- or (S)-isomer. If that compound has one isomeric form predominant over the other, for example, 80% (S)-isomer and 20% (R)-isomer, the enantiomeric purity of the compound with respect to the (S)-isomeric form is 80%. The enantiomeric purity of a compound is determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or Pirkle's reagents, or derivatization of a compounds using a chiral compound such as Mosher's acid followed by chromatography or nuclear magnetic resonance spectroscopy.

In some embodiments, the enantiomerically enriched composition has a higher potency with respect to therapeutic utility per unit mass than does the racemic mixture of that composition. Enantiomers can be isolated from mixtures by methods known to those skilled in the art, including chiral high pressure liquid chromatography (HPLC) and the formation and crystallization of chiral salts; preferred enantiomers can be prepared by asymmetric syntheses. See, for example, Jacques, et al., Enantiomers, Racemates and Resolutions, Wiley Interscience, New York (1981); E. L. Eliel, Stereochemistry of Carbon Compounds, McGraw-Hill, New York (1962); and E. L. Eliel and S. H. Wilen, Stereochemistry of Organic Compounds, Wiley-Interscience, New York (1994).

The terms “enantiomerically enriched” and “non-racemic,” as used herein, refer to compositions in which the percent by weight of one enantiomer is greater than the amount of that one enantiomer in a control mixture of the racemic composition (e.g., greater than 1:1 by weight). For example, an enantiomerically enriched preparation of the (S)-enantiomer, means a preparation of the compound having greater than 50% by weight of the (S)-enantiomer relative to the (R)-enantiomer, such as for example, and without limitation, at least 75% by weight, at least 80% by weight, or the like. In some embodiments, the enrichment is significantly greater than 80% by weight, providing a “substantially enantiomerically enriched” or a “substantially non-racemic” preparation, which refers to preparations of compositions which have at least 85% by weight of one enantiomer relative to other enantiomer, such as at least 90% by weight, at least 95% by weight, or the like. The terms “enantiomerically pure” or “substantially enantiomerically pure” refer to compositions comprising at least 98% of a single enantiomer and less than 2% of the opposite enantiomer.

“Moiety” refers to a specific segment or functional group of a molecule. Chemical moieties are often recognized chemical entities embedded in or appended to a molecule.

“Tautomers” are structurally distinct isomers that interconvert by tautomerization. “Tautomerization” is a form of isomerization and includes prototropic or proton-shift tautomerization, which is considered a subset of acid-base chemistry. “Prototropic tautomerization” or “proton-shift tautomerization” involves the migration of a proton accompanied by changes in bond order, often the interchange of a single bond with an adjacent double bond. Where tautomerization is possible (e.g., in solution), a chemical equilibrium of tautomers is reached. An example of tautomerization is keto-enol tautomerization. A specific example of keto-enol tautomerization is the interconversion of pentane-2,4-dione and 4-hydroxypent-3-en-2-one tautomers. Another example of tautomerization is phenol-keto tautomerization. A specific example of phenol-keto tautomerization is the interconversion of pyridin-4-ol and pyridin-4(1H)-one tautomers.

A “leaving group or atom” is any group or atom that will, under selected reaction conditions, cleave from the starting material, thus promoting reaction at a specified site. Examples of such groups, unless otherwise specified, include halogen atoms and mesyloxy, p-nitrobenzensulphonyloxy and tosyloxy groups.

“Protecting group” is intended to mean a group that selectively blocks one or more reactive sites in a multifunctional compound such that a chemical reaction is carried out selectively on another unprotected reactive site and the group can then be readily removed or deprotected after the selective reaction is complete. A variety of protecting groups are disclosed, for example, in T. H. Greene and P. G. M. Wuts, Protective Groups in Organic Synthesis, 3rd Edition, John Wiley & Sons, New York (1999).

“Solvate” refers to a compound in physical association with one or more molecules of a pharmaceutically acceptable solvent.

“Substituted” means that the referenced group may have one or more hydrogen atoms replaced by one or more additional groups, radicals or moieties individually and independently selected from, for example, acyl, alkyl, alkylaryl, cycloalkyl, aralkyl, aryl, carbohydrate, carbonate, heteroaryl, heterocycloalkyl, hydroxamate, hydroxy, alkoxy, aryloxy, mercapto, alkylthio, arylthio, cyano, halo, carbonyl, ester, thiocarbonyl, isocyanato, thiocyanato, isothiocyanato, nitro, oxo, perhaloalkyl, perfluoroalkyl, phosphate, silyl, sulfinyl, sulfonyl, sulfonamidyl, sulfoxyl, sulfonate, urea, and amino, including mono- and di-substituted amino groups, and protected derivatives thereof. The substituents themselves may be substituted, for example, a cycloalkyl substituent may itself have a halide substituent at one or more of its ring carbons. The term “optionally substituted” means optional substitution with the specified groups, radicals or moieties.

“Sulfanyl” refers to groups that include —S-(optionally substituted alkyl), —S-(optionally substituted aryl), —S-(optionally substituted heteroaryl) and —S-(optionally substituted heterocycloalkyl).

“Sulfinyl” refers to groups that include —S(O)—H, —S(O)-(optionally substituted alkyl), —S(O)-(optionally substituted amino), —S(O)-(optionally substituted aryl), —S(O)-(optionally substituted heteroaryl) and —S(O)-(optionally substituted heterocycloalkyl).

“Sulfonyl” refers to groups that include —S(O₂)—H, —S(O₂)-(optionally substituted alkyl), —S(O₂)-(optionally substituted amino), —S(O₂)-(optionally substituted aryl), —S(O₂)-(optionally substituted heteroaryl), and —S(O₂)-(optionally substituted heterocycloalkyl).

“Sulfonamidyl” or “sulfonamido” refers to a —S(═O)₂—NRR radical, where each R is selected independently from the group consisting of hydrogen, alkyl, cycloalkyl, aryl, heteroaryl (bonded through a ring carbon) and heteroalicyclic (bonded through a ring carbon). The R groups in —NRR of the —S(═O)₂—NRR radical may be taken together with the nitrogen to which it is attached to form a 4-, 5-, 6- or 7-membered ring. A sulfonamido group is optionally substituted by one or more of the substituents described for alkyl, cycloalkyl, aryl, heteroaryl, respectively.

“Sulfoxyl” refers to a —S(═O)₂OH radical.

“Sulfonate” refers to a —S(═O)₂—OR radical, where R is selected from the group consisting of alkyl, cycloalkyl, aryl, heteroaryl (bonded through a ring carbon) and heteroalicyclic (bonded through a ring carbon). A sulfonate group is optionally substituted on R by one or more of the substituents described for alkyl, cycloalkyl, aryl, heteroaryl, respectively.

The terms “active pharmaceutical ingredient” and “drug” include the compounds of formula (I), formula (1), formula (2), formula (10), formula (11), formula (3), formulas 1001-1003, or formulas 1004-1008, that allosterically modulate β₂-adrenoceptor, as described herein. The terms “active pharmaceutical ingredient” and “drug” may also include other additional compounds that either bind to and activate, including allosterically binding to and activating, β₂-adrenoceptor (i.e., β₂-adrenoceptor agonists) or bind to and inhibit, including allosterically binding to and inhibiting, β₂-adrenoceptor (i.e., β₂-adrenoceptor antagonists), as described herein.

Compounds of the disclosure also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form” and “polymorph” are intended to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

As used herein, the terms “administer,” “administration,” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

The terms “combination,” “pharmaceutical combination,” “co-administration,” “co-administering,” “administered in combination with,” “administering in combination with,” “simultaneous,” and “concurrent,” as used herein, encompass administration of two or more active pharmaceutical ingredients to a subject so that both active pharmaceutical ingredients and/or their metabolites are present in the subject at the same time. Co-administration includes concurrent administration and sequential administration. The simultaneous administration of two or more separate compositions is an example of concurrent co-administration. Another example of concurrent co-administration is the administration of a single composition in which two or more active pharmaceutical ingredients are present. The administration of two or more separate compositions at different times is an example of sequential co-administration.

Generally, the terms “modulate” and “modulation” refer to a change in biological activity for a biological molecule (e.g., a protein, gene, peptide, antibody, and the like), where such change may relate to an increase in biological activity (e.g., increased activity, agonism, activation, expression, upregulation, and/or increased expression) or decrease in biological activity (e.g., decreased activity, antagonism, suppression, deactivation, downregulation, and/or decreased expression) for the biological molecule. The terms “allosteric modulator” or “allosteric modulation,” as used herein, refer to a compound that does not bind to an orthosteric binding site of β₂-adrenoceptor, but instead acts elsewhere to modulate β₂-adrenoceptor function or activity. Binding by an allosteric modulator does not trigger a functional activity in the absence of the orthosteric ligand (e.g., a β₂-adrenoceptor agonist or β₂-adrenoceptor antagonist). A positive allosteric modulator (PAM) is a compound that amplifies or augments the effect of an orthosteric β₂-adrenoceptor agonist or β₂-adrenoceptor antagonist. A negative allosteric modulator (NAM) is a compound that reduces or diminishes the effect of an orthosteric β₂-adrenoceptor agonist or β₂-adrenoceptor antagonist.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, the manner of administration, etc., which can readily be determined by one of ordinary skill in the art. The term also applies to a dose that induces a particular response in target cells (e.g., the reduction of platelet adhesion and/or cell migration). The specific dose varies depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the terms “treat,” “treatment,” and/or “treating” may refer to the management of a disease, disorder, or pathological condition, or symptom thereof with the intent to cure, ameliorate, stabilize, and/or control the disease, disorder, pathological condition or symptom thereof. Regarding control of the disease, disorder, or pathological condition more specifically, “control” may include the absence of condition progression, as assessed by the response to the methods recited herein, where such response may be complete (e.g., placing the disease in remission) or partial (e.g., lessening or ameliorating any symptoms associated with the condition). As used herein, the terms “prevent,” “preventing,” and/or “prevention” may refer to reducing the risk of developing a disease, disorder, or pathological condition.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The terms “subject” and “patient” are used interchangeably herein to refer to a warm blooded animal such as a mammal, preferably a human, or a human child, which is afflicted with, or has the potential to be afflicted with one or more diseases and/or conditions described herein.

The term “synergistic,” “synergistic effect,” “synergistic combination,” or “synergism” as used herein, generally refers to an effect such that the one or more effects of the combination of components is greater than the one or more effects of each component alone, or they can be greater than the sum of the one or more effects of each component alone. The synergistic effect can be greater than about 10%, 20%, 30%, 40%, 50%, 60%, 75%, 100%, 110%, 120%, 150%, 200%, 250%, 350%, or 500% or more than the effect on a subject with one of the components alone, or the additive effects of each of the components when administered individually. The effect can be any of the measurable effects described herein. Synergistic combinations of the disclosure include a combination of i.) one or more active pharmaceutical ingredients of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof; and ii.) a β₂-adrenoceptor agonist or a β₂-adrenoceptor antagonist. Advantageously, such synergy between the active pharmaceutical ingredients when combined, may allow for the use of smaller doses of one or both active pharmaceutical ingredients, may provide greater efficacy at the same doses, and may prevent or delay the build-up of multi-drug resistance and/or toxicity. The combination index (CI) method of Chou and Talalay may be used to determine the synergy, additive or antagonism effect of the agents used in combination. When the CI value is less than 1, there is synergy between the drugs used in the combination; when the CI value is equal to 1, there is an additive effect between the drugs used in the combination and when CI value is more than 1, there is an antagonistic effect. The synergistic effect may be attained by co-formulating the active pharmaceutical ingredients of the pharmaceutical combination. The synergistic effect may be attained by administering two or more active pharmaceutical ingredients as separate formulations administered simultaneously or sequentially.

The terms “QD,” “qd,” or “q.d.” mean quaque die, once a day, or once daily. The terms “BID,” “bid,” or “b.i.d.” mean bis in die, twice a day, or twice daily. The terms “TID,” “tid,” or “t.i.d.” mean ter in die, three times a day, or three times daily. The terms “QID,” “qid,” or “q.i.d.” mean quater in die, four times a day, or four times daily.

“Pharmaceutically acceptable” refers to those compounds, materials, compositions, and/or dosage forms which are, within the scope of sound medical judgment, suitable for contact with the tissues of human beings and non-human animals without excessive toxicity, irritation, allergic response, or other adverse complications commensurate with a reasonable benefit/risk ratio.

“Pharmaceutically acceptable carrier” or “pharmaceutically acceptable excipient” or “physiologically compatible” carrier or carrier medium is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient(s), its use in the therapeutic compositions of the disclosure is contemplated.

The term “pharmaceutically acceptable salt” refers to salts derived from a variety of organic and inorganic counter ions known in the art. Pharmaceutically acceptable acid addition salts is formed with inorganic acids and organic acids. Preferred inorganic acids from which salts is derived include, for example, hydrochloric acid, hydrobromic acid, sulfuric acid, nitric acid and phosphoric acid. Preferred organic acids from which salts is derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid and salicylic acid. Pharmaceutically acceptable base addition salts is formed with inorganic and organic bases. Inorganic bases from which salts is derived include, for example, sodium, potassium, lithium, ammonium, calcium, magnesium, iron, zinc, copper, manganese and aluminum. Organic bases from which salts is derived include, for example, primary, secondary, and tertiary amines, substituted amines including naturally occurring substituted amines, cyclic amines and basic ion exchange resins. Specific examples include isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts. The term “cocrystal” refers to a molecular complex derived from a number of cocrystal formers known in the art. Unlike a salt, a cocrystal typically does not involve hydrogen transfer between the cocrystal and the drug, and instead involves intermolecular interactions, such as hydrogen bonding, aromatic ring stacking, or dispersive forces, between the cocrystal former and the drug in the crystal structure.

Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids.

Base addition salts include those derived from inorganic bases such as ammonium and alkali and alkaline earth metal hydroxides, carbonates, bicarbonates, and the like, as well as salts derived from basic organic compounds such as aliphatic and aromatic amines, aliphatic diamines, hydroxy alkamines, and the like. Such bases useful in preparing the salts of this disclosure thus include ammonium hydroxide, potassium carbonate, sodium bicarbonate, calcium hydroxide, methylamine, diethylamine, ethylenediamine, cyclohexylamine, ethanolamine and the like.

In addition to pharmaceutically-acceptable salts, other salts are included within the scope of this disclosure. They may serve as intermediates in the purification of the compounds, in the preparation of other salts, or in the identification and characterization of the compounds or intermediates.

The pharmaceutically acceptable salts of compounds of the present disclosure can also exist as various solvates, such as with water, methanol, ethanol, dimethylformamide, ethyl acetate and the like. Mixtures of such solvates can also be prepared. The source of such solvates can be from the solvent of crystallization, inherent in the solvent of preparation or crystallization, or adventitious to such solvent. Such solvates are also within the scope of the present disclosure.

“Prodrug” is intended to describe a compound that may be converted under physiological conditions or by solvolysis to a biologically active pharmaceutical ingredient described herein. Thus, the term “prodrug” refers to a precursor of a biologically active pharmaceutical ingredient that is pharmaceutically acceptable. A prodrug may be inactive when administered to a subject, but is converted in vivo to an active pharmaceutical ingredient, for example, by hydrolysis. The prodrug compound often offers the advantages of solubility, tissue compatibility or delayed release in a mammalian organism (see, e.g., Bundgaard, H., Design of Prodrugs (1985) (Elsevier, Amsterdam). The term “prodrug” is also intended to include any covalently bonded carriers, which release the active pharmaceutical ingredient in vivo when administered to a subject. Prodrugs of an active pharmaceutical ingredient, as described herein, may be prepared by modifying functional groups present in the active pharmaceutical ingredient in such a way that the modifications are cleaved, either in routine manipulation or in vivo, to yield the active pharmaceutical ingredient. Prodrugs include, for example, compounds wherein a hydroxy, amino or mercapto group is bonded to any group that, when the prodrug of the active pharmaceutical ingredient is administered to a mammalian subject, cleaves to form a free hydroxy, free amino or free mercapto group, respectively. Examples of prodrugs include, but are not limited to, acetates, formates and benzoate derivatives of an alcohol, various ester derivatives of a carboxylic acid, or acetamide, formamide and benzamide derivatives of an amine functional group in the active pharmaceutical ingredient.

Unless otherwise stated, the chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by ¹³C- or ¹⁴C-enriched carbons, are within the scope of this disclosure.

When ranges are used herein to describe, for example, physical or chemical properties such as, molecular weight, chemical formulae, molar ratios, etc., all combinations and subcombinations of ranges and specific embodiments therein are intended to be included. As used herein, the terms “about” and “around,” and the like, are used herein to modify a numerical value and indicate a defined range around that value. Use of the term “about” when referring to a number or a numerical range means that the number or numerical range referred to is an approximation within experimental variability (or within statistical experimental error), and thus the number or numerical range may vary. Without wishing to be bound to any particular value, the variation can be from 0% to 15%, from 0% to 10%, or from 0% to 5% of the stated number or numerical range. For example, if “X” is the value, “about X” or “around X” generally indicates, without limitation, a value from 0.90X to 1.10X. In some embodiments, a reference to “about X” indicates, without limitation, at least the values X, 0.90X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, and 1.10X. Thus, “about X” is intended to disclose, e.g., “0.98X.” In some embodiments, when “about” is applied to the beginning of a numerical range, it applies to both ends of the range. For example, “from about 6 to 8.5” is equivalent to “from about 6 to about 8.5.” In some embodiments, when “about” is applied to the first value of a set of values, it applies to all values in that set. For example, “about 7, 9, or 11%” is equivalent to “about 7%, about 9%, or about 11%.”

The term “comprising” (and related terms such as “comprise” or “comprises” or “having” or “including”) includes those embodiments such as, for example, an embodiment of any composition of matter, method or process that “consist of” or “consist essentially of” the described features.

All other terms used in the description of the present disclosure have their art recognized meanings.

As will be apparent to anyone skilled in the art, the compounds of the present disclosure may have one or more chiral centers, and in that case, exist in various stereoisomeric forms. The compounds of the present disclosure encompass all such optical isomers, diastereomers and enantiomers. The compounds are normally prepared as a racemic mixture or racemate and can conveniently be used as such, but individual enantiomers can be isolated or synthesized by conventional techniques if so desired. Such racemates and individual enantiomers and mixtures thereof form part of the present disclosure.

It is well known in the art how to prepare and isolate such optically active forms from a mixture of enantiomers. Specific stereoisomers can be prepared by stereospecific synthesis using enantiomerically pure or enantiomerically enriched starting materials. The specific stereoisomers of either starting materials or products can be resolved and recovered by techniques known in the art, such as resolution of racemic forms, normal, reverse-phase, and chiral chromatography, recrystallization, enzymatic resolution, or fractional recrystallization of addition salts formed by reagents used for that purpose. Useful methods of resolving and recovering specific stereoisomers described in Eliel, E. L.; Wilen, S. H. Stereochemistry of Organic Compounds; Wiley: New York, 1994, and Jacques, J, et al. Enantiomers, Racemates, and Resolutions; Wiley: New York, 1981, each incorporated by reference herein in their entireties.

For the avoidance of doubt, it is intended herein that particular features (for example integers, characteristics, values, uses, diseases, formulae, compounds or groups) described in conjunction with a particular aspect, embodiment or example of the disclosure are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The disclosure is not restricted to any details of any disclosed embodiments. The disclosure extends to any novel one, or novel combination, of the features disclosed in this specification (including any accompanying claims, abstract and drawings), or to any novel one, or any novel combination, of the steps of any method or process so disclosed.

### II. Compounds

In one aspect, the disclosure relates to a compound of formula (I), or a pharmaceutically acceptable salt, solvate, hydrate, cocrystal, or prodrug thereof:

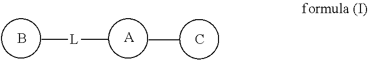

wherein in formula (I):
   A is an optionally substituted 6-membered heteroaryl or optionally substituted 6-membered heterocyclyl, provided that the optionally substituted 6-membered heteroaryl or optionally substituted 6-membered heterocyclyl comprises two or more nitrogen atoms;
   B is an optionally substituted monocyclic aryl or optionally substituted monocyclic or bicyclic heteroaryl;
   C is an optionally substituted heteroaryl or optionally substituted cycloalkyl;
   L is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —CR^{a}═N—NR^{a}—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and
   R^{a} is each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl.

In some embodiments, A is selected from optionally substituted pyrimidine, optionally substituted pyridazine, optionally substituted pyrazine, and optionally substituted piperazine.

In some embodiments, A is selected from:

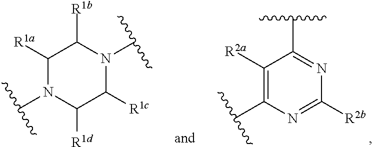

wherein R^{1a}, R^{1b}, R^{1c}, R^{1d}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)OR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

In some embodiments, B is optionally substituted aryl, optionally substituted pyridyl, or optionally substituted quinoxaline.

In some embodiments, C is optionally substituted 3- to 7-membered cycloalkyl, optionally substituted pyrrole, optionally substituted imidazole, optionally substituted pyrazole, or optionally substituted triazole.

In another aspect, the disclosure relates to a compound of formula (1), or a pharmaceutically acceptable salt, solvate, hydrate, cocrystal, or prodrug thereof:

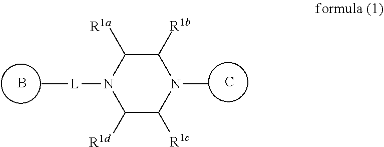

wherein in formula (I):
   B is an optionally substituted monocyclic heteroaryl; and
   C is an optionally substituted 3- to 7-membered cycloalkyl.

In another aspect, the disclosure relates to a compound of formula (2), or a pharmaceutically acceptable salt, solvate, hydrate, cocrystal, or prodrug thereof:

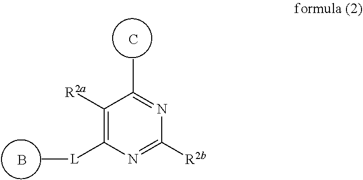

wherein in formula (2):
   B is an optionally substituted monocyclic aryl; and
   C is an optionally heteroaryl.

In some embodiments, B is selected from.

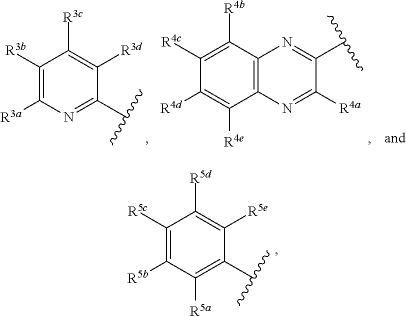

wherein R^{3a}, R^{3b}, R^{3c}, R^{3d}, R^{4a}, R^{4b}, R^{4c}, R^{4d}, R^{4e}, R^{5a}, R^{5b}, R^{5c}, R^{5d}, and R^{5e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})Re, —N(R^{a})S(O)ₜR^{a}, —C(O)N(R^{a})S(O)R^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

In another aspect, the disclosure relates to a compound of formula (10), or a pharmaceutically acceptable salt, solvate, hydrate, cocrystal, or prodrug thereof:

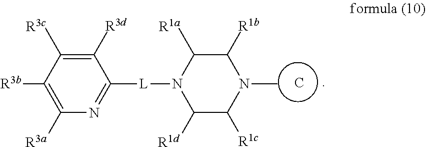

In another aspect, the disclosure relates to a compound of formula (11), or a pharmaceutically acceptable salt, solvate, hydrate, cocrystal, or prodrug thereof:

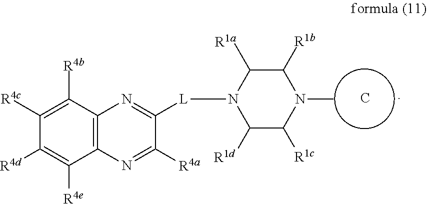

In some embodiments, R^{1a}, R^{1b}, and R^{1d} are each H. In some embodiments, R^{1c} is substituted C₁₋₆ alkyl. In some embodiments, R^{1c} is substituted ethyl. In some embodiments, R^{1c} is —(CH₂)₂—OH.

In some embodiments, R^{3a}, R^{3b}, R^{3c}, and R^{3d} are each H.

In some embodiments, R^{4b}, R^{4c}, R^{4d}, and R^{4e} are each H. In some embodiments, R^{4a} is C₁₋₆ alkyl. In some embodiments, R⁴, is —CH₃.

In some embodiments, C is selected from:

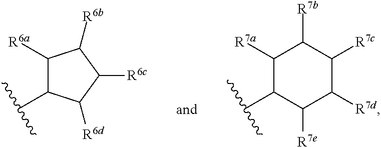

wherein R^{6a}, R^{6b}, R^{6c}, R^{6d}, R^{7a}, R^{7b}, R^{7c}, R^{7d}, and R^{7e} are each independently selected from H, OH, halo, cyano, fluoroalkyl, trifluoromethyl, trifluoromethoxy, nitro, trimethylsilanyl, —OR^{a}, —SR, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

In some embodiments, C is:

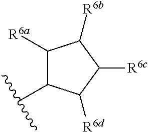

R^{6a}, R^{6b}, R^{6c}, and R^{6d} are each H.

In some embodiments, C is:

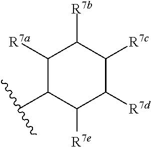

and
R^{7a}, R^{7b}, R^{7c}, R^{7d}, and R^{7e} are each H.

In some embodiments, L is —(CH₂)₁₋₆—. In some embodiments, L is —(CH₂)—.

In another aspect, the disclosure relates to a compound of formula (3), or a pharmaceutically acceptable salt, solvate, hydrate, cocrystal, or prodrug thereof:

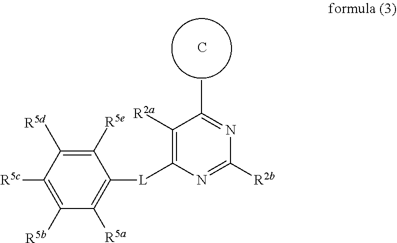

wherein in formula (3):
   B is an optionally substituted monocyclic aryl; and
   C is an optionally heteroaryl.

In some embodiments, R^{2a} and R^{2b} are each H. In some embodiments, R^{5b}, R^{5c}, R^{5d}, and R^{5e} are each H. In some embodiments, R^{5a} is —OR^{a}. In some embodiments, R^{5a} is —OH.

In some embodiments, C is:

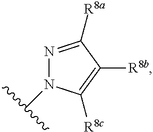

wherein R^{8a}, R^{8b}, and R^{8c} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{a}, —N(R^{a})S(O)ₜR^{a}, —C(O)N(R^{a})S(O)ₜR^{a}, —S(O)ₜOR^{a}, —S(O)N(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

In some embodiments, R^{8a} and R^{8c} are each H. In some embodiments, R^{8b} is C₁₋₆ alkyl. In some embodiments, R^{8b} is —CH₃.

In some embodiments, L is —CR^{a}═N—NR^{a}—. In some embodiments, L is —CH═N—NH—.

In some embodiments, the compound is an allosteric activator of the β₂-adrenoceptor.

In some embodiments, the compound is an allosteric inhibitor of the β₂-adrenoceptor.

In some embodiments, the compound of any one of formula (I), formula (1), formula (2), formula (10), formula (11), formula (3), or a pharmaceutically acceptable salt, solvate, hydrate, cocrystal, or prodrug thereof, is not a compound of any one of formula 1001-1008:

| Compound # | Structure |
|---|---|
| 1001 | 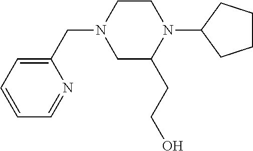 [SMILES: OCCC1CN(Cc2ccccn2)CCN1C1CCCC1] |
| 1002 | 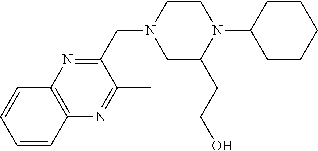 [SMILES: Cc1nc2ccccc2nc1CN1CCN(C2CCCCC2)C(CCO)C1] |
| 1003 | 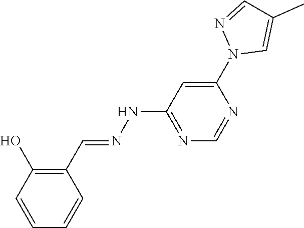 [SMILES: Cc1cnn(-c2cc(N/N=C/c3ccccc3O)ncn2)c1] |
| 1004 | 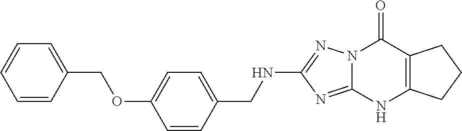 [SMILES: O=c1c2c([nH]c3nc(NCc4ccc(OCc5ccccc5)cc4)nn13)CCC2] |
| 1005 | 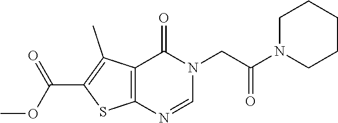 [SMILES: COC(=O)c1sc2ncn(CC(=O)N3CCCCC3)c(=O)c2c1C] |
| 1006 | 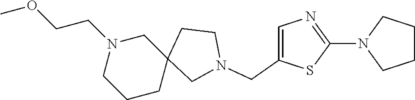 [SMILES: COCCN1CCCC2(CCN(Cc3cnc(N4CCCC4)s3)C2)C1] |
| 1007 | 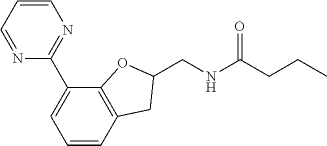 [SMILES: CCCC(=O)NCC1Cc2cccc(-c3ncccn3)c2O1] |
| 1008 | 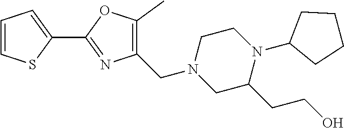 [SMILES: Cc1oc(-c2cccs2)nc1CN1CCN(C2CCCC2)C(CCO)C1] |

### III. Methods of Treatment

The compounds and compositions described herein can be used in methods for treating diseases and/or disorders. In some embodiments, the compounds and compositions described herein can be used in methods for treating diseases or disorders alleviated by allosterically modulating the activity of β₂-adrenoceptor. In some embodiments, the compounds and compositions described herein can be used in methods for treating diseases alleviated by allosterically activating β₂-adrenoceptor. In some embodiments, the compounds and compositions described herein can be used in methods for treating alleviated by allosterically inhibiting β₂-adrenoceptor.

In one aspect, the disclosure includes a method of treating a disease or disorder alleviated by allosterically modulating β₂-adrenoceptor in a patient in need thereof, the method comprising administering to the patient a therapeutically effective amount of a compound of formula (I), formula (1), formula (2), formula (10), formula (11), or formula (3), or a pharmaceutically acceptable salt, solvate, hydrate, cocrystal, or prodrug thereof. In some embodiments, the method comprises administering to the patient a therapeutically effective amount of a compound of formula 1001, formula 1002, or formula 1003, or a pharmaceutically acceptable salt, solvate, hydrate, cocrystal, or prodrug thereof:

| Compound # | Structure |
|---|---|
| 1001 | 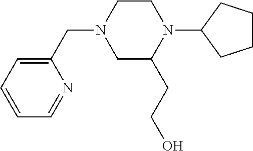 [SMILES: OCCC1CN(Cc2ccccn2)CCN1C1CCCC1] |
| 1002 | 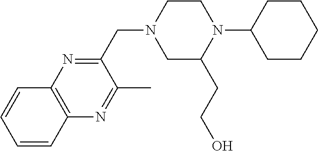 [SMILES: Cc1nc2ccccc2nc1CN1CCN(C2CCCCC2)C(CCO)C1] |
| 1003 | 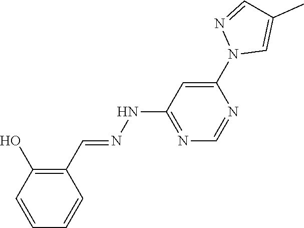 [SMILES: Cc1cnn(-c2cc(N/N=C/c3ccccc3O)ncn2)c1] |

In some embodiments, the method of treating a disease or disorder alleviated by allosterically modulating β₂-adrenoceptor in a patient in need thereof comprises administering to the patient a therapeutically effective amount of a compound of formula (I), formula (1), formula (2), formula (10), formula (11), formula (3), or formulas 1001-1003, or a pharmaceutically acceptable salt, solvate, hydrate, cocrystal, or prodrug thereof.

In other embodiments, the method of treating a disease or disorder alleviated by allosterically modulating β₂-adrenoceptor in a patient in need thereof comprises administering to the patient a therapeutically effective amount of a compound of formulas 1004-1008, or a pharmaceutically acceptable salt, solvate, hydrate, cocrystal, or prodrug thereof:

| Compound # | Structure |
|---|---|
| 1004 | 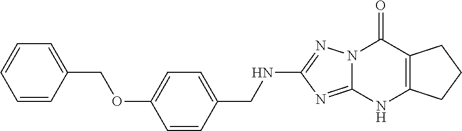 [SMILES: O=c1c2c([nH]c3nc(NCc4ccc(OCc5ccccc5)cc4)nn13)CCC2] |
| 1005 | 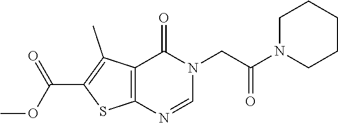 [SMILES: COC(=O)c1sc2ncn(CC(=O)N3CCCCC3)c(=O)c2c1C] |
| 1006 | 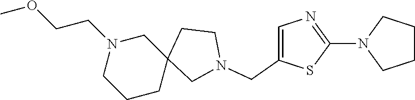 [SMILES: COCCN1CCCC2(CCN(Cc3cnc(N4CCCC4)s3)C2)C1] |
| 1007 | 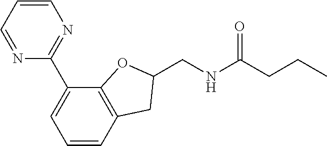 [SMILES: CCCC(=O)NCC1Cc2cccc(-c3ncccn3)c2O1] |
| 1008 | 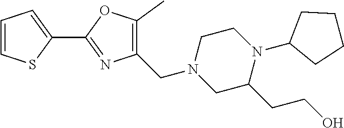 [SMILES: Cc1oc(-c2cccs2)nc1CN1CCN(C2CCCC2)C(CCO)C1] |

In some embodiments, the method of treating a disease or disorder alleviated by allosterically modulating β₂-adrenoceptor in a patient in need thereof comprises administering to the patient a therapeutically effective amount of a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof.

In one embodiment, the method of treating a disease or disorder comprises allosterically activating β₂-adrenoceptor. In some embodiments, the disease or disorder is an inflammatory disease. In some embodiments, the inflammatory disease is selected from the group consisting of rheumatoid arthritis, a cardiovascular disease, multiple sclerosis, inflammatory bowel disease, chronic obstructive pulmonary disease (COPD), asthma, acute respiratory distress syndrome (ARDS), and acute lung injury (ALI). In some embodiments, the inflammatory disease is asthma or COPD. In some embodiments, the method comprises administering to the patient a therapeutically effective amount of a compound of formula 1001, or a pharmaceutically acceptable salt, solvate, hydrate, cocrystal, or prodrug thereof:

| Compound # | Structure |
|---|---|
| 1001 | 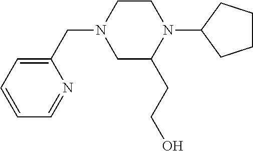 [SMILES: OCCC1CN(Cc2ccccn2)CCN1C1CCCC1] |

In one embodiment, the method of treating a disease or disorder comprises allosterically inhibiting β₂-adrenoceptor. In some embodiments, the disease or disorder is a cardiovascular disease. In some embodiments, the cardiovascular disease is selected from the group consisting of coronary obstructions, coronary artery disease (CAD), angina, heart attack, cardiac arrhythmia, cardiomyopathy, myocardial infarction, cardiac failure, high blood pressure, heart valve disease, and heart failure. Non-limiting examples of cardiomyopathy include ischemic cardiomyopathy, dilated cardiomyopathy, restrictive cardiomyopathy, and hypertrophic cardiomyopathy. Non-limiting examples of heart failure include chronic heart failure; acute decompensated heart failure; high-output heart failure; left-sided heart failure; right-sided heart failure; biventricular heart failure; heart failure due to reduced ejection fraction (HFrEF), also referred to as “systolic heart failure;” heart failure with preserved ejection fraction (HFpEF), also referred to as “diastolic heart failure;” and congestive failure. In some embodiments, the cardiovascular disease is cardiomyopathy or heart failure. In some embodiments, the cardiovascular disease is cardiomyopathy. In some embodiments, the cardiovascular disease is heart failure. In some embodiments, the cardiovascular disease is congestive heart failure. In some embodiments, the method comprises administering to the patient a therapeutically effective amount of a compound of formula 1002 or formula 1003, or a pharmaceutically acceptable salt, solvate, hydrate, cocrystal, or prodrug thereof:

| Compound # | Structure |
|---|---|
| 1002 | 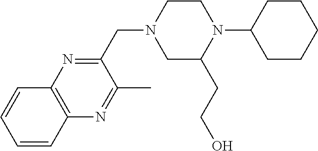 [SMILES: Cc1nc2ccccc2nc1CN1CCN(C2CCCCC2)C(CCO)C1] |
| 1003 | 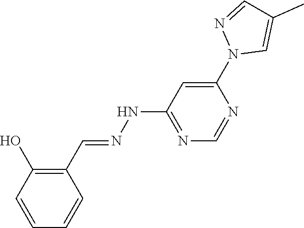 [SMILES: Cc1cnn(-c2cc(N/N=C/c3ccccc3O)ncn2)c1] |

In some embodiments, the method of treating a disease or disorder alleviated by allosterically modulating β₂-adrenoceptor in a patient in need thereof comprises administering to the patient a therapeutically effective amount of a compound of formula (I), formula (1), formula (2), formula (10), formula (11), formula (3), or formula 1001, or a pharmaceutically acceptable salt, solvate, hydrate, cocrystal, or prodrug thereof, thereby allosterically activating β₂-adrenoceptor in the patient. In some embodiments, the method of treating a disease or disorder alleviated by allosterically modulating β₂-adrenoceptor in a patient in need thereof comprises administering to the patient a therapeutically effective amount of a compound of formula 1001, or a pharmaceutically acceptable salt, solvate, hydrate, cocrystal, or prodrug thereof, thereby allosterically activating β₂-adrenoceptor in the patient. In some embodiments, the disease or disorder is an inflammatory disease. In some embodiments, the inflammatory disease is selected from the group consisting of chronic obstructive pulmonary disease (COPD), asthma, acute respiratory distress syndrome (ARDS), and acute lung injury (ALI). In some embodiments, the inflammatory disease is asthma or COPD.

In some embodiments, the method of treating a disease or disorder alleviated by allosterically activating β₂-adrenoceptor in a patient in need thereof comprises administering to the patient a therapeutically effective amount of a compound of formula (I), formula (1), formula (2), formula (10), formula (11), formula (3), or formula 1001, or a pharmaceutically acceptable salt, solvate, hydrate, cocrystal, or prodrug thereof. In some embodiments, the method of treating a disease or disorder alleviated by allosterically activating β₂-adrenoceptor in a patient in need thereof comprises administering to the patient a therapeutically effective amount of a compound of formula 1001, or a pharmaceutically acceptable salt, solvate, hydrate, cocrystal, or prodrug thereof. In some embodiments, the disease or disorder is an inflammatory disease. In some embodiments, the inflammatory disease is selected from the group consisting of chronic obstructive pulmonary disease (COPD), asthma, acute respiratory distress syndrome (ARDS), and acute lung injury (ALI). In some embodiments, the inflammatory disease is asthma or COPD.

In some embodiments, the method of treating a disease or disorder alleviated by allosterically modulating β₂-adrenoceptor in a patient in need thereof comprises administering to the patient a therapeutically effective amount of a compound of formula (I), formula (1), formula (2), formula (10), formula (11), formula (3), formula 1002, or formula 1003, or a pharmaceutically acceptable salt, solvate, hydrate, cocrystal, or prodrug thereof, thereby allosterically inhibiting β₂-adrenoceptor in the patient. In some embodiments, the method of treating a disease or disorder alleviated by allosterically modulating β₂-adrenoceptor in a patient in need thereof comprises administering to the patient a therapeutically effective amount of a compound of formula 1002, or a pharmaceutically acceptable salt, solvate, hydrate, cocrystal, or prodrug thereof, thereby allosterically inhibiting β₂-adrenoceptor in the patient. In some embodiments, the method of treating a disease or disorder alleviated by allosterically modulating β₂-adrenoceptor in a patient in need thereof comprises administering to the patient a therapeutically effective amount of a compound of formula 1003, or a pharmaceutically acceptable salt, solvate, hydrate, cocrystal, or prodrug thereof, thereby allosterically inhibiting β₂-adrenoceptor in the patient. In some embodiments, the disease or disorder is an cardiovascular disease. In some embodiments, the cardiovascular disease is selected from the group consisting of cardiac arrhythmia, cardiomyopathy, myocardial infarction, heart valve disease, and heart failure. In some embodiments, the cardiovascular disease is heart failure.

In some embodiments, the method of treating a disease or disorder alleviated by allosterically inhibiting β₂-adrenoceptor in a patient in need thereof comprises administering to the patient a therapeutically effective amount of a compound of formula (I), formula (1), formula (2), formula (10), formula (11), formula (3), formula 1002, or formula 1003, or a pharmaceutically acceptable salt, solvate, hydrate, cocrystal, or prodrug thereof. In some embodiments, the method of treating a disease or disorder alleviated by allosterically inhibiting β₂-adrenoceptor in a patient in need thereof comprises administering to the patient a therapeutically effective amount of a compound of formula 1002, or a pharmaceutically acceptable salt, solvate, hydrate, cocrystal, or prodrug thereof. In some embodiments, the method of treating a disease or disorder alleviated by allosterically inhibiting β₂-adrenoceptor in a patient in need thereof comprises administering to the patient a therapeutically effective amount of a compound of formula 1003, or a pharmaceutically acceptable salt, solvate, hydrate, cocrystal, or prodrug thereof. In some embodiments, the disease or disorder is an cardiovascular disease. In some embodiments, the cardiovascular disease is selected from the group consisting of cardiac arrhythmia, cardiomyopathy, myocardial infarction, heart valve disease, and heart failure. In some embodiments, the cardiovascular disease is heart failure.

In one aspect, the disclosure includes a method of treating a disease or disorder alleviated by allosterically modulating β₂-adrenoceptor in a patient in need thereof, the method comprising administering to the patient a therapeutically effective amount of an allosteric activator of the β₂-adrenoceptor, wherein the allosteric activator of the β₂-adrenoceptor is a compound of formula (I), formula (1), formula (2), formula (10), formula (11), formula (3), or formula 1001, or a pharmaceutically acceptable salt, solvate, hydrate, cocrystal, or prodrug thereof. In some embodiments, the method of treating a disease or disorder alleviated by allosterically modulating β₂-adrenoceptor in a patient in need thereof comprises administering to the patient a therapeutically effective amount of an allosteric activator of the β₂-adrenoceptor, wherein the allosteric activator of the β₂-adrenoceptor is a compound of formula 1001, or a pharmaceutically acceptable salt, solvate, hydrate, cocrystal, or prodrug thereof.

In one aspect, the disclosure includes a method of treating a disease or disorder alleviated by allosterically modulating β₂-adrenoceptor in a patient in need thereof, the method comprising administering to the patient a therapeutically effective amount of an allosteric inhibitor of the β₂-adrenoceptor, wherein the allosteric inhibitor of the β₂-adrenoceptor is a compound of formula (I), formula (1), formula (2), formula (10), formula (11), formula (3), formula 1002, or formula 1003, or a pharmaceutically acceptable salt, solvate, hydrate, cocrystal, or prodrug thereof. In some embodiments, the method of treating a disease or disorder alleviated by allosterically modulating β₂-adrenoceptor in a patient in need thereof comprises administering to the patient a therapeutically effective amount of an allosteric inhibitor of the β₂-adrenoceptor, wherein the allosteric inhibitor of the β₂-adrenoceptor is a compound of formula 1002, or a pharmaceutically acceptable salt, solvate, hydrate, cocrystal, or prodrug thereof. In some embodiments, the method of treating a disease or disorder alleviated by allosterically modulating β₂-adrenoceptor in a patient in need thereof comprises administering to the patient a therapeutically effective amount of an allosteric inhibitor of the β₂-adrenoceptor, wherein the allosteric inhibitor of the β₂-adrenoceptor is a compound of formula 1003, or a pharmaceutically acceptable salt, solvate, hydrate, cocrystal, or prodrug thereof.

In some embodiments, the patient or subject is a mammal, such as a human. In an embodiment, the patient or subject is a human. In an embodiment, the patient or subject is a companion animal. In an embodiment, the patient or subject is a canine, feline, or equine.

In some embodiments, the compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, is administered in a unit dosage form.

In some embodiments, the unit dosage form of the compound of formula (1), formula (1), formula (2), formula (10), formula (11), formula (3), formulas 1001-1003, or formulas 1004-1008, or a pharmaceutically acceptable salt, solvate, hydrate, cocrystal, or prodrug thereof, comprises a physiologically compatible carrier medium.

In some embodiments, the compound of formula 1001, or a pharmaceutically acceptable salt, solvate, hydrate, cocrystal, or prodrug thereof, is administered in a unit dosage form. In some embodiments, the unit dosage form of the compound of formula 1001, or a pharmaceutically acceptable salt, solvate, hydrate, cocrystal, or prodrug thereof, comprises a physiologically compatible carrier medium.

In some embodiments, the compound of formula 1002, or a pharmaceutically acceptable salt, solvate, hydrate, cocrystal, or prodrug thereof, is administered in a unit dosage form. In some embodiments, the unit dosage form of the compound of formula 1002, or a pharmaceutically acceptable salt, solvate, hydrate, cocrystal, or prodrug thereof, comprises a physiologically compatible carrier medium.

In some embodiments, the compound of formula 1003, or a pharmaceutically acceptable salt, solvate, hydrate, cocrystal, or prodrug thereof, is administered in a unit dosage form. In some embodiments, the unit dosage form of the compound of formula 1003, or a pharmaceutically acceptable salt, solvate, hydrate, cocrystal, or prodrug thereof, comprises a physiologically compatible carrier medium.

In some embodiments, the method of treating a disease or disorder alleviated by allosterically modulating β₂-adrenoceptor in a patient in need thereof comprises administering to the patient a therapeutically effective amount of a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, wherein the compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, is administered in a unit dosage form. In some embodiments, the unit dosage form comprises a physiologically compatible carrier medium.

In some embodiments, the method of treating a disease or disorder alleviated by allosterically activating β₂-adrenoceptor in a patient in need thereof comprises administering to the patient a therapeutically effective amount of a compound of formula (I), formula (1), formula (2), formula (10), formula (11), formula (3), or formula 1001, or a pharmaceutically acceptable salt, solvate, hydrate, cocrystal, or prodrug thereof, wherein the compound of formula (I), formula (1), formula (2), formula (10), formula (11), formula (3), or formula 1001, or a pharmaceutically acceptable salt, solvate, hydrate, cocrystal, or prodrug thereof, is administered in a unit dosage form. In some embodiments, the unit dosage form comprises a physiologically compatible carrier medium.

In some embodiments, the method of treating a disease or disorder alleviated by allosterically inhibiting β₂-adrenoceptor in a patient in need thereof comprises administering to the patient a therapeutically effective amount of a compound of formula (I), formula (1), formula (2), formula (10), formula (11), formula (3), formula 1002, or formula 1003, or a pharmaceutically acceptable salt, solvate, hydrate, cocrystal, or prodrug thereof, wherein the compound of formula (I), formula (1), formula (2), formula (10), formula (11), formula (3), formula 1002, or formula 1003, or a pharmaceutically acceptable salt, solvate, hydrate, cocrystal, or prodrug thereof, is administered in a unit dosage form. In some embodiments, the unit dosage form comprises a physiologically compatible carrier medium.

### IV. Pharmaceutical Compositions

In one aspect, the disclosure provides a pharmaceutical composition comprising any of the β₂-adrenoceptor modulators described herein, or a pharmaceutically acceptable salt, solvate, hydrate, cocrystal, or prodrug thereof, and a physiologically compatible carrier medium. In one embodiment, the disclosure provides a pharmaceutical composition comprising a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a physiologically compatible carrier medium for use in the treatment of the diseases and conditions described herein.

In one embodiment, the disclosure provides a pharmaceutical composition comprising a β₂-adrenoceptor modulator of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a physiologically compatible carrier medium. In one embodiment, the disclosure provides a pharmaceutical composition comprising an allosteric activator of the β₂-adrenoceptor of formula (I), formula (1), formula (2), formula (10), formula (11), formula (3), or formula 1001, or a pharmaceutically acceptable salt, solvate, hydrate, cocrystal, or prodrug thereof, and a physiologically compatible carrier medium. In one embodiment, the disclosure provides a pharmaceutical composition comprising an allosteric inhibitor of the β₂-adrenoceptor of formula (1), formula (1), formula (2), formula (10), formula (11), formula (3), formula 1002, or formula 1003, or a pharmaceutically acceptable salt, solvate, hydrate, cocrystal, or prodrug thereof, and a physiologically compatible carrier medium.

In one embodiment, the disclosure provides a pharmaceutical composition comprising a compound of formula (1), or a pharmaceutically acceptable salt, solvate, hydrate, cocrystal, or prodrug thereof, and a physiologically compatible carrier medium. In one embodiment, the disclosure provides a pharmaceutical composition comprising a compound of formula (1), or a pharmaceutically acceptable salt, solvate, hydrate, cocrystal, or prodrug thereof, and a physiologically compatible carrier medium. In one embodiment, the disclosure provides a pharmaceutical composition comprising a compound of formula (2), or a pharmaceutically acceptable salt, solvate, hydrate, cocrystal, or prodrug thereof, and a physiologically compatible carrier medium. In one embodiment, the disclosure provides a pharmaceutical composition comprising a compound of formula (10), or a pharmaceutically acceptable salt, solvate, hydrate, cocrystal, or prodrug thereof, and a physiologically compatible carrier medium. In one embodiment, the disclosure provides a pharmaceutical composition comprising a compound of formula (11), or a pharmaceutically acceptable salt, solvate, hydrate, cocrystal, or prodrug thereof, and a physiologically compatible carrier medium. In one embodiment, the disclosure provides a pharmaceutical composition comprising a compound of formula (3), or a pharmaceutically acceptable salt, solvate, hydrate, cocrystal, or prodrug thereof, and a physiologically compatible carrier medium.

In one embodiment, the disclosure provides a pharmaceutical composition comprising a compound of formula 1001, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1002, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1003, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1004, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1005, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1006, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1007, or a pharmaceutically acceptable salt, solvate, hydrate, cocrystal, or prodrug thereof, and a physiologically compatible carrier medium. In one embodiment, the disclosure provides a pharmaceutical composition comprising a compound of formula 1008, or a pharmaceutically acceptable salt, solvate, hydrate, cocrystal, or prodrug thereof, and a physiologically compatible carrier medium.

Any compound disclosed herein, for example a compound of formula (I), formula (1), formula (2), formula (10), formula (11), formula (3), formulas 1001-1003, or formulas 1004-1008, or pharmaceutically acceptable salt thereof, may be administered to a subject by itself, or in the form of a pharmaceutical composition. Pharmaceutical compositions comprising the compounds of the disclosure may be manufactured by means of conventional mixing, dissolving, granulating, dragee-making, levigating, emulsifying, encapsulating, entrapping or lyophilizing processes. Pharmaceutical compositions may be formulated in conventional manner using one or more physiological acceptable carriers, diluents, excipients, or auxiliaries which facilitate processing of the compounds into preparations which can be used pharmaceutically. A specific formulation method will be dependent upon the route of administration chosen.

The pharmaceutical compositions are typically formulated to provide a therapeutically effective amount of a compound of formula (I), formula (1), formula (2), formula (10), formula (11), formula (3), formulas 1001-1003, or formulas 1004-1008, or a fragment, derivative, conjugate, variant, radioisotope-labeled complex, or biosimilar thereof, or pharmaceutically acceptable salts, prodrugs, solvates, or hydrates thereof, as the active ingredients. Where desired, the pharmaceutical compositions contain a pharmaceutically acceptable salt and/or coordination complex of one or more of the active ingredients.

Where desired, other active pharmaceutical ingredient(s) may be mixed into a preparation or two or more components of the combination may be formulated into separate preparations for use in combination separately or at the same time. A kit containing the components of the combination, formulated into separate preparations for said use, is also provided by the disclosure. Accordingly, in some embodiments, the disclosure provides a pharmaceutical composition comprising two or more active pharmaceutical ingredients.

In some embodiments, the disclosure provides a pharmaceutical composition comprising a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, a physiologically compatible carrier medium, and, optionally, one or more other active pharmaceutical ingredients. In some embodiments, the one or more other active pharmaceutical ingredients is a β₂-adrenoceptor agonist or a β₂-adrenoceptor antagonist. Non-limiting examples of β₂-adrenoceptor agonists include isoproterenol (ISO), albuterol, salmeterol, salbutamol, and formoterol.

In some embodiments, the concentration of each active pharmaceutical ingredient (e.g., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each active pharmaceutical ingredient (e.g., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently greater than, for example, 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of each active pharmaceutical ingredient (e.g., a compound of formula (1), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v, or v/v of the pharmaceutical composition.

In some embodiments, the concentration of each active pharmaceutical ingredient (e.g., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v, or v/v of the pharmaceutical composition.

In some embodiments, the amount or dose of each active pharmaceutical ingredient (i.e., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently equal to or less than about 10 g, about 9.5 g, about 9.0 g, about 8.5 g, about 8.0 g, about 7.5 g, about 7.0 g, about 6.5 g, about 6.0 g, about 5.5 g, about 5.0 g, about 4.5 g, about 4.0 g, about 3.5 g, about 3.0 g, about 2.5 g, about 2.0 g, about 1.5 g, about 1.0 g, about 0.95 g, about 0.9 g, about 0.85 g, about 0.8 g, about 0.75 g, about 0.7 g, about 0.65 g, about 0.6 g, about 0.55 g, about 0.5 g, about 0.45 g, about 0.4 g, about 0.35 g, about 0.3 g, about 0.25 g, about 0.2 g, about 0.15 g, about 0.1 g, about 0.09 g, about 0.08 g, about 0.07 g, about 0.06 g, about 0.05 g, about 0.04 g, about 0.03 g, about 0.02 g, about 0.01 g, about 0.009 g, about 0.008 g, about 0.007 g, about 0.006 g, about 0.005 g, about 0.004 g, about 0.003 g, about 0.002 g, about 0.001 g, about 0.0009 g, about 0.0008 g, about 0.0007 g, about 0.0006 g, about 0.0005 g, about 0.0004 g, about 0.0003 g, about 0.0002 g, or about 0.0001 g.

In some embodiments, the amount or dose of each active pharmaceutical ingredient (i.e., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and the optionally included other active pharmaceutical ingredient(s)) provided in a pharmaceutical composition of the disclosure is independently more than about 0.0001 g, about 0.0002 g, about 0.0003 g, about 0.0004 g, about 0.0005 g, about 0.0006 g, about 0.0007 g, about 0.0008 g, about 0.0009 g, about 0.001 g, about 0.0015 g, about 0.002 g, about 0.0025 g, about 0.003 g, about 0.0035 g, about 0.004 g, about 0.0045 g, about 0.005 g, about 0.0055 g, about 0.006 g, about 0.0065 g, about 0.007 g, about 0.0075 g, about 0.008 g, about 0.0085 g, about 0.009 g, about 0.0095 g, about 0.01 g, about 0.015 g, about 0.02 g, about 0.025 g, about 0.03 g, about 0.035 g, about 0.04 g, about 0.045 g, about 0.05 g, about 0.055 g, about 0.06 g, about 0.065 g, about 0.07 g, about 0.075 g, about 0.08 g, about 0.085 g, about 0.09 g, about 0.095 g, about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, about 1.5 g, about 2 g, about 2.5, about 3 g, about 3.5, about 4 g, about 4.5 g, about 5 g, about 5.5 g, about 6 g, about 6.5 g, about 7 g, about 7.5 g, about 8 g, about 8.5 g, about 9 g, about 9.5 g, or about 10 g.

Each of the compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and the optionally included other active pharmaceutical ingredient(s)) is effective over a wide dosage range. For example, in the treatment of adult humans, dosages independently ranging from about 0.01 to about 1000 mg, from about 0.5 to about 100 mg, from about 1 to about 50 mg per day, and from about 5 to about 40 mg per day are examples of dosages that may be used. The exact dosage will depend upon the route of administration, the form in which the compound is administered, the gender and age of the subject to be treated, the body weight of the subject to be treated, and the preference and experience of the attending physician.

For pharmaceutical compositions containing more than one active pharmaceutical ingredient (e.g., one active pharmaceutical ingredient selected from a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof; and another active pharmaceutical ingredient selected from a β₂-adrenoceptor agonist or a β₂-adrenoceptor antagonist), the molar ratio of two active pharmaceutical ingredients in the pharmaceutical compositions is in the range from 10:1 to 1:10, preferably from 2.5:1 to 1:2.5, and more preferably about 1:1. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20. In an embodiment, the weight ratio of the molar ratio of two active pharmaceutical ingredients in the pharmaceutical compositions is selected from the group consisting of 20:1, 19:1, 18:1, 17:1, 16:1, 15:1, 14:1, 13:1, 12:1, 11:1, 10:1, 9:1, 8:1, 7:1, 6:1, 5:1, 4:1,3:1,2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, and 1:20.

In an embodiment, the pharmaceutical compositions described herein, such as those compositions comprising one active pharmaceutical ingredient (e.g., a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof) or those compositions comprising more than one active pharmaceutical ingredient (e.g., one active pharmaceutical ingredient selected from a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof; and another active pharmaceutical ingredient selected from a β₂-adrenoceptor agonist or a β₂-adrenoceptor antagonist), are for use in the treatment of an inflammatory disease or a cardiovascular disease.

In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, and a physiologically compatible carrier medium is for the treatment of an inflammatory disease. In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, and a physiologically compatible carrier medium is for the treatment of inflammatory bowel disease, chronic obstructive pulmonary disease (COPD), asthma, acute respiratory distress syndrome (ARDS), and acute lung injury (ALI). In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, and a physiologically compatible carrier medium is for the treatment of asthma or COPD.

In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, a physiologically compatible carrier medium, and a β₂-adrenoceptor agonist is for the treatment of an inflammatory disease. In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, a physiologically compatible carrier medium, and a β₂-adrenoceptor agonist is for the treatment of chronic obstructive pulmonary disease (COPD), asthma, acute respiratory distress syndrome (ARDS), and acute lung injury (ALI). In one embodiment, the pharmaceutical compositions described herein comprising a compound of formula (I), formula (1), formula (2), formula (10), formula (11), formula (3), or formula 1001, or a pharmaceutically acceptable salt, solvate, hydrate, cocrystal, or prodrug thereof, a physiologically compatible carrier medium, and a β₂-adrenoceptor agonist is for the treatment of asthma or COPD.

In one embodiment, the pharmaceutical compositions described herein comprising a compound of formula 1001, or a pharmaceutically acceptable salt, solvate, hydrate, cocrystal, or prodrug thereof, and a physiologically compatible carrier medium is for the treatment of an inflammatory disease. In one embodiment, the pharmaceutical compositions described herein comprising a compound of formula 1001, or a pharmaceutically acceptable salt, solvate, hydrate, cocrystal, or prodrug thereof, and a physiologically compatible carrier medium is for the treatment of chronic obstructive pulmonary disease (COPD), asthma, acute respiratory distress syndrome (ARDS), and acute lung injury (ALI). In one embodiment, the pharmaceutical compositions described herein comprising a compound of formula 1001, or a pharmaceutically acceptable salt, solvate, hydrate, cocrystal, or prodrug thereof, and a physiologically compatible carrier medium is for the treatment of asthma or COPD.

In one embodiment, the pharmaceutical compositions described herein comprising a compound of formula 1001, or a pharmaceutically acceptable salt, solvate, hydrate, cocrystal, or prodrug thereof, a physiologically compatible carrier medium, and a β₂-adrenoceptor agonist is for the treatment of an inflammatory disease. In one embodiment, the pharmaceutical compositions described herein comprising a compound of formula 1001, or a pharmaceutically acceptable salt, solvate, hydrate, cocrystal, or prodrug thereof, a physiologically compatible carrier medium, and a β₂-adrenoceptor agonist is for the treatment of chronic obstructive pulmonary disease (COPD), asthma, acute respiratory distress syndrome (ARDS), and acute lung injury (ALI). In one embodiment, the pharmaceutical compositions described herein comprising a compound of formula 1001, or a pharmaceutically acceptable salt, solvate, hydrate, cocrystal, or prodrug thereof, a physiologically compatible carrier medium, and a β₂-adrenoceptor agonist is for the treatment of asthma. In one embodiment, the pharmaceutical compositions described herein comprising a compound of formula 1001, or a pharmaceutically acceptable salt, solvate, hydrate, cocrystal, or prodrug thereof, a physiologically compatible carrier medium, and a β₂-adrenoceptor agonist is for the treatment of COPD.

In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, and a physiologically compatible carrier medium is for the treatment of a cardiovascular disease. In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, and a physiologically compatible carrier medium is for the treatment of cardiac arrhythmia, cardiomyopathy, myocardial infarction, heart valve disease, and heart failure. In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, and a physiologically compatible carrier medium is for the treatment of heart failure.

In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, a physiologically compatible carrier medium, and a β₂-adrenoceptor antagonist is for the treatment of a cardiovascular disease. In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, a physiologically compatible carrier medium, and a β₂-adrenoceptor antagonist is for the treatment of cardiac arrhythmia, cardiomyopathy, myocardial infarction, heart valve disease, and heart failure. In one embodiment, the pharmaceutical compositions described herein comprising a compound of formula (I), formula (1), formula (2), formula (10), formula (11), formula (3), formula 1002, or formula 1003, or a pharmaceutically acceptable salt, solvate, hydrate, cocrystal, or prodrug thereof, a physiologically compatible carrier medium, and a β₂-adrenoceptor antagonist is for the treatment of heart failure.

In one embodiment, the pharmaceutical compositions described herein comprising a compound of formula 1002, or a pharmaceutically acceptable salt, solvate, hydrate, cocrystal, or prodrug thereof, and a physiologically compatible carrier medium is for the treatment of a cardiovascular disease. In one embodiment, the pharmaceutical compositions described herein comprising a compound of formula 1002, or a pharmaceutically acceptable salt, solvate, hydrate, cocrystal, or prodrug thereof, and a physiologically compatible carrier medium is for the treatment of cardiac arrhythmia, cardiomyopathy, myocardial infarction, heart valve disease, and heart failure. In one embodiment, the pharmaceutical compositions described herein comprising a compound of formula 1002, or a pharmaceutically acceptable salt, solvate, hydrate, cocrystal, or prodrug thereof, and a physiologically compatible carrier medium is for the treatment of heart failure.

In one embodiment, the pharmaceutical compositions described herein comprising a compound of formula 1002, or a pharmaceutically acceptable salt, solvate, hydrate, cocrystal, or prodrug thereof, a physiologically compatible carrier medium, and a β₂-adrenoceptor antagonist is for the treatment of a cardiovascular disease. In one embodiment, the pharmaceutical compositions described herein comprising a compound of formula 1002, or a pharmaceutically acceptable salt, solvate, hydrate, cocrystal, or prodrug thereof, a physiologically compatible carrier medium, and a β₂-adrenoceptor antagonist is for the treatment of cardiac arrhythmia, cardiomyopathy, myocardial infarction, heart valve disease, and heart failure. In one embodiment, the pharmaceutical compositions described herein comprising a compound of formula 1002, or a pharmaceutically acceptable salt, solvate, hydrate, cocrystal, or prodrug thereof, a physiologically compatible carrier medium, and a β₂-adrenoceptor antagonist is for the treatment of heart failure.

In one embodiment, the pharmaceutical compositions described herein comprising a compound of formula 1003, or a pharmaceutically acceptable salt, solvate, hydrate, cocrystal, or prodrug thereof, and a physiologically compatible carrier medium is for the treatment of a cardiovascular disease. In one embodiment, the pharmaceutical compositions described herein comprising a compound of formula 1003, or a pharmaceutically acceptable salt, solvate, hydrate, cocrystal, or prodrug thereof, and a physiologically compatible carrier medium is for the treatment of cardiac arrhythmia, cardiomyopathy, myocardial infarction, heart valve disease, and heart failure. In one embodiment, the pharmaceutical compositions described herein comprising a compound of formula 1003, or a pharmaceutically acceptable salt, solvate, hydrate, cocrystal, or prodrug thereof, and a physiologically compatible carrier medium is for the treatment of heart failure.

In one embodiment, the pharmaceutical compositions described herein comprising a compound of formula 1003, or a pharmaceutically acceptable salt, solvate, hydrate, cocrystal, or prodrug thereof, a physiologically compatible carrier medium, and a β₂-adrenoceptor antagonist is for the treatment of a cardiovascular disease. In one embodiment, the pharmaceutical compositions described herein comprising a compound of formula 1003, or a pharmaceutically acceptable salt, solvate, hydrate, cocrystal, or prodrug thereof, a physiologically compatible carrier medium, and a β₂-adrenoceptor antagonist is for the treatment of cardiac arrhythmia, cardiomyopathy, myocardial infarction, heart valve disease, and heart failure. In one embodiment, the pharmaceutical compositions described herein comprising a compound of formula 1003, or a pharmaceutically acceptable salt, solvate, hydrate, cocrystal, or prodrug thereof, a physiologically compatible carrier medium, and a β₂-adrenoceptor antagonist is for the treatment of heart failure.

Described below are non-limiting pharmaceutical compositions and methods for preparing the same.

### Pharmaceutical Compositions for Oral Administration,

In some embodiments, the disclosure provides a pharmaceutical composition for oral administration containing a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a pharmaceutical excipient suitable for administration. In other embodiments, the disclosure provides a pharmaceutical composition for oral administration containing a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, a pharmaceutical excipient suitable for administration, and one or more additional active pharmaceutical ingredient.

In some embodiments, the pharmaceutical composition may be a solid pharmaceutical composition suitable for oral consumption. In some embodiments, the pharmaceutical composition may be a liquid pharmaceutical composition suitable for oral consumption.

Pharmaceutical compositions of the disclosure suitable for oral administration can be presented as discrete dosage forms, such as capsules, sachets, tablets, liquids, or aerosol sprays each containing a predetermined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms can be prepared by any of the methods of pharmacy, but all methods include the step of bringing the active ingredient(s) into association with the carrier, which constitutes one or more necessary ingredients. In general, the compositions are prepared by uniformly and intimately admixing the active ingredient(s) with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

The disclosure further encompasses anhydrous pharmaceutical compositions and dosage forms since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the disclosure can be prepared using anhydrous or low moisture containing ingredients and low moisture or low humidity conditions. Pharmaceutical compositions and dosage forms of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

Each of the compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, used as active ingredients can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the disclosure to provide tablets that disintegrate when exposed to an aqueous environment. Too much of a disintegrant may produce tablets which disintegrate in the bottle. Too little may be insufficient for disintegration to occur, thus altering the rate and extent of release of the active ingredients from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, silicified microcrystalline cellulose, or mixtures thereof. A lubricant can optionally be added in an amount of less than about 0.5% or less than about 1% (by weight) of the pharmaceutical composition.

When aqueous suspensions and/or elixirs are desired for oral administration, the active pharmaceutical ingredient(s) may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl-lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; camitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogs thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In an embodiment, the composition may include a solubilizer to ensure good solubilization and/or dissolution of the compound of the present disclosure and to minimize precipitation of the compound of the present disclosure. This can be especially important for compositions for non-oral use—e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals and alkaline earth metals. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid.

### Pharmaceutical Compositions for Injection

In some embodiments, the disclosure provides a pharmaceutical composition for injection containing a compound of a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, according to the disclosure, and a pharmaceutical excipient suitable for injection. Components and amounts of agents in the compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol and liquid polyethylene glycol (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, and thimerosal.

Sterile injectable solutions are prepared by incorporating an active pharmaceutical ingredient of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, or a combination of active pharmaceutical ingredients (e.g., one or more compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a β₂-adrenoceptor agonist or β₂-adrenoceptor antagonist) in the required amounts in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical Delivery

In some embodiments, the disclosure provides a pharmaceutical composition for transdermal delivery containing a compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, according to the disclosure, and a pharmaceutical excipient suitable for transdermal delivery

Compositions of the present disclosure can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present disclosure employs transdermal delivery devices (“patches”). Such transdermal patches may be used to provide continuous or discontinuous infusion of an active pharmaceutical ingredient or combination of active pharmaceutical ingredients in controlled amounts, either with or without another active pharmaceutical ingredient.

The construction and use of transdermal patches for the delivery of pharmaceutical agents is well known in the art. See, e.g., U.S. Pat. Nos. 5,023,252; 4,992,445; and 5,001,139, the entirety of which are incorporated herein by reference. Such patches may be constructed for continuous, pulsatile, or on demand delivery of pharmaceutical agents.

### Pharmaceutical Compositions for Inhalation

In some embodiments, the disclosure provides a pharmaceutical composition for inhalation or insufflation delivery containing an active pharmaceutical ingredient of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, or a combination of active pharmaceutical ingredients (e.g., one or more compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a β₂-adrenoceptor agonist or β₂-adrenoceptor antagonist), according to the disclosure. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner. Dry powder inhalers may also be used to provide inhaled delivery of the compositions.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; and Pratt and Taylor, eds., Principles of Drug Action, Third Edition, Churchill Livingston, N.Y., 1990, each of which is incorporated by reference herein in its entirety.

Administration of an active pharmaceutical ingredient of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof; or a combination of active pharmaceutical ingredients (e.g., one or more compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a β₂-adrenoceptor agonist or β₂-adrenoceptor antagonist); or a pharmaceutical composition thereof, can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. The active pharmaceutical ingredient or combination of active pharmaceutical ingredients can also be administered intraadiposally or intrathecally.

Exemplary parenteral administration forms include solutions or suspensions of active compound in sterile aqueous solutions, for example, aqueous propylene glycol or dextrose solutions. Such dosage forms can be suitably buffered, if desired.

### V. Kits

The disclosure also provides kits. The kits include an active pharmaceutical ingredient of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, or a combination of active pharmaceutical ingredients (e.g., one or more compounds of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof, and a β₂-adrenoceptor agonist or β₂-adrenoceptor antagonist), or pharmaceutical compositions thereof, either alone or in combination in suitable packaging, that can include instructions for use, discussion of clinical studies and listing of side effects. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In selected embodiments, an active pharmaceutical ingredient or combination of active pharmaceutical ingredients are provided as separate compositions in separate containers within the kit. In selected embodiments, an active pharmaceutical ingredient or combination of active pharmaceutical ingredients are provided as a single composition within a container in the kit.

Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in selected embodiments, be marketed directly to the consumer.

In some embodiments, the disclosure provides a kit comprising a composition comprising a therapeutically effective amount of an active pharmaceutical ingredient of formula (I), formula (1), formula (2), formula (10), formula (11), formula (3), formulas 1001-1003, or formulas 1004-1008, or a fragment, derivative, conjugate, variant, radioisotope-labeled complex, or biosimilar thereof, or pharmaceutically acceptable salts, prodrugs, solvates, or hydrates thereof. These compositions are typically pharmaceutical compositions.

In some embodiments, the disclosure provides a kit comprising a composition comprising a therapeutically effective amount of a combination of active pharmaceutical ingredients (e.g., one or more compounds of formula (I), formula (1), formula (2), formula (10), formula (11), formula (3), formulas 1001-1003, or formulas 1004-1008, or a fragment, derivative, conjugate, variant, radioisotope-labeled complex, or biosimilar thereof, or pharmaceutically acceptable salts, prodrugs, solvates, or hydrates thereof, and a β₂-adrenoceptor agonist or β₂-adrenoceptor antagonist). These compositions are typically pharmaceutical compositions. The kit is for co-administration of the combination of active pharmaceutical ingredients, either simultaneously or separately.

In some embodiments, the kits described above are preferably for use in the treatment of the diseases and or disorders described herein. In some embodiments, the kits are for use in the treatment of an inflammatory disease. In some embodiments, the kits are for use in the treatment of chronic obstructive pulmonary disease (COPD), asthma, acute respiratory distress syndrome (ARDS), and acute lung injury (ALI). In some embodiments, the kits are for use in the treatment of asthma. In some embodiments, the kits are for use in the treatment of COPD. In some embodiments, the kits are for use in the treatment of a cardiovascular disease. In some embodiments, the kits are for use in the treatment of cardiac arrhythmia, cardiomyopathy, myocardial infarction, heart valve disease, and heart failure. In some embodiments, the kits are for use in the treatment of heart failure.

In addition to the formulations previously described, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver the compounds of the disclosure. Certain organic solvents such as dimethylsulfoxide also may be employed, although usually at the cost of greater toxicity. Additionally, the compounds may be delivered using a sustained-release system, such as semipermeable matrices of solid polymers containing the therapeutic agent. A variety of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed. As the compounds of the disclosure may contain charged side chains or termini, they may be included in any of the above-described formulations as the free acids or bases or as pharmaceutically acceptable salts. Pharmaceutical salts tend to be more soluble in aqueous and other protic solvents than are the corresponding free acid or base forms.

The methods of the present disclosure will normally include medical follow-up to determine the therapeutic or prophylactic effect brought about in the patient undergoing treatment with the compound(s) and/or composition(s) described herein.

### VI. Dosages and Dosing Regimens

The amount of the compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), to be administered using the methods herein, will be dependent on the human or mammal being treated, the severity of the disorder or condition, the rate of administration, the disposition of the compounds (and optional additional active pharmaceutical ingredients), and the discretion of the prescribing physician. However, an effective dosage of each is in the range of about 0.001 to about 100 mg per kg body weight per day, such as about 1 to about 35 mg/kg/day, in single or divided doses. For a 70 kg human, this would amount to about 0.05 to 7 g/day, such as about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day. The dosage of the compound formula (1), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), may be provided in units of mg/kg of body mass, or in mg/m² of body surface area.

In some embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is administered in a single dose. Such administration may be by inhalation or injection, e.g., intravenous injection, in order to introduce the active pharmaceutical ingredient(s) quickly. However, other routes, including the oral route, may be used as appropriate. A single dose of the active pharmaceutical ingredient(s) may also be used for treatment of an acute condition.

In some embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered in multiple doses. Dosing may be once, twice, three times, four times, five times, six times, or more than six times per day. Dosing may be once a month, once every two weeks, once a week, or once every other day. In other embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered about once per day to about 6 times per day. In some embodiments the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered once daily. In other embodiments the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered twice daily. In some embodiments the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered three times daily.

Administration of the active pharmaceutical ingredients of the disclosure may continue as long as necessary. In some embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered for more than 1, 2, 3, 4, 5, 6, 7, 14, or 28 days. In some embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered for less than 28, 14, 7, 6, 5, 4, 3, 2, or 1 day. In some embodiments, the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, is administered chronically on an ongoing basis, e.g., for the treatment of chronic effects. In another embodiment the administration of the compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), or pharmaceutical compositions thereof, continues for less than about 7 days. In other embodiments the administration continues for more than about 6, 10, 14, 28 days, two months, six months, or one year. In some cases, continuous dosing is achieved and maintained as long as necessary.

In some embodiments, an effective dosage of a compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is in the range of about 1 mg to about 50 mg, about 5 mg to about 45 mg, about 10 mg to about 40 mg, about 15 mg to about 35 mg, about 20 mg to about 30 mg, about 23 mg to about 28 mg, about 1 mg to about 500 mg, about 10 mg to about 300 mg, about 20 mg to about 250 mg, about 25 mg to about 200 mg, about 10 mg to about 200 mg, about 20 mg to about 150 mg, about 30 mg to about 120 mg, about 10 mg to about 90 mg, about 20 mg to about 80 mg, about 30 mg to about 70 mg, about 40 mg to about 60 mg, about 45 mg to about 55 mg, about 48 mg to about 52 mg, about 50 mg to about 150 mg, about 60 mg to about 140 mg, about 70 mg to about 130 mg, about 80 mg to about 120 mg, about 90 mg to about 110 mg, about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, about 198 to about 202 mg, or about 198 to about 207 mg.

In some embodiments, an effective dosage of a compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg.

In some embodiments, an effective dosage of a compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is in the range of about 0.01 mg/kg to about 200 mg/kg, or about 0.1 to 100 mg/kg, or about 1 to 50 mg/kg.

In some embodiments, an effective dosage of a compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is in the range of about 0.01 mg/kg to about 0.7 mg/kg, about 0.07 mg/kg to about 0.65 mg/kg, about 0.15 mg/kg to about 0.6 mg/kg, about 0.2 mg/kg to about 0.5 mg/kg, about 0.3 mg/kg to about 0.45 mg/kg, about 0.3 mg/kg to about 0.4 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg to about 1.6 mg/kg, about 1.35 mg/kg to about 1.5 mg/kg, about 1.4 mg/kg to about 1.45 mg/kg, about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of a compound formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients), is about 0.35 mg/kg, about 0.4 mg/kg, about 0.7 mg/kg, about 1 mg/kg, about 1.4 mg/kg, about 1.8 mg/kg, about 2.1 mg/kg, about 2.5 mg/kg, about 2.85 mg/kg, about 3.2 mg/kg, or about 3.6 mg/kg.

In some instances, dosage levels below the lower limit of the aforesaid ranges may be more than adequate, while in other cases still larger doses may be employed without causing any harmful side effect, e.g., by dividing such larger doses into several small doses for administration throughout the day. As those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

An effective amount of the compound of formula (I), formula (1), formula (2), formula (10), formula (11), formula (3), formulas 1001-1003, or formulas 1004-1008, or a pharmaceutically acceptable salt, solvate, hydrate, cocrystal, or prodrug thereof (and optional additional active pharmaceutical ingredients, may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In some embodiments, the compositions described herein further include controlled-release, sustained release, or extended-release therapeutic dosage forms for administration of the compounds described herein, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

The disclosure will be further described in the following embodiments, which do not limit the scope of the disclosure described in the claims.

Embodiment 1. A compound of formula (I), or a pharmaceutically acceptable salt, solvate, hydrate, cocrystal, or prodrug thereof:

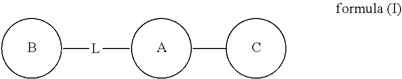

wherein in formula (I):
   A is an optionally substituted 6-membered heteroaryl or optionally substituted 6-membered heterocyclyl, provided that the optionally substituted 6-membered heteroaryl or optionally substituted 6-membered heterocyclyl comprises two or more nitrogen atoms;
   B is an optionally substituted monocyclic aryl or optionally substituted monocyclic or bicyclic heteroaryl;
   C is an optionally substituted heteroaryl or optionally substituted cycloalkyl;
   L is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR⁸¹C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —CR^{a}═N—NR^{a}—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and
   R^{a} is each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl.

Embodiment 2. The compound of Embodiment 1, wherein A is selected from optionally substituted pyrimidine, optionally substituted pyridazine, optionally substituted pyrazine, and optionally substituted piperazine.

Embodiment 3. The compound of Embodiment 2, wherein A is selected from:

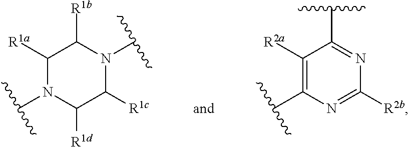

wherein R^{1a}, R^{1b}, R^{1c}, R^{1d}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)R^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

Embodiment 4. The compound of any one of Embodiments 1-3, wherein B is optionally substituted aryl, optionally substituted pyridyl, or optionally substituted quinoxaline.

Embodiment 5. The compound of any one of Embodiments 1-4, wherein C is optionally substituted 3- to 7-membered cycloalkyl, optionally substituted pyrrole, optionally substituted imidazole, optionally substituted pyrazole, or optionally substituted triazole.

Embodiment 6. The compound of any one of Embodiments 3-5, or a pharmaceutically acceptable salt, solvate, hydrate, cocrystal, or prodrug thereof, wherein the compound has a structure according to formula (1):

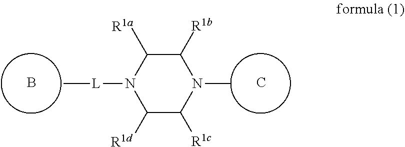

wherein in formula (1):
   B is an optionally substituted monocyclic heteroaryl; and
   C is an optionally substituted 3- to 7-membered cycloalkyl.

Embodiment 7. The compound of any one of Embodiments 3-5, or a pharmaceutically acceptable salt, solvate, hydrate, cocrystal, or prodrug thereof, wherein the compound has a structure according to formula (2):

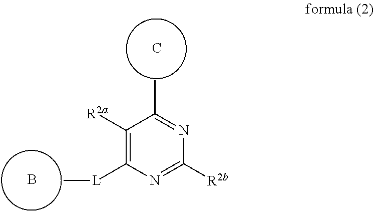

wherein in formula (2):
   B is an optionally substituted monocyclic aryl; and
   C is an optionally heteroaryl.

Embodiment 8. The compound of any one of Embodiments 1-7, wherein B is selected from:

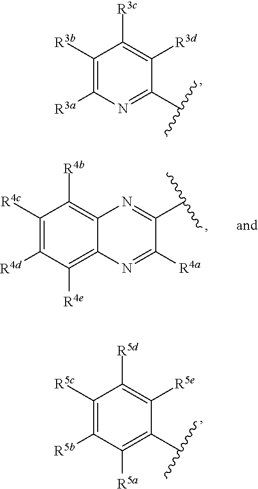

wherein R^{3a}, R^{3b}, R^{3c}, R^{3d}, R^{4a}, R^{4b}, R^{4c}, R^{4d}, R^{4e}, R^{5a}, R^{5b}, R^{5c}, R^{5d}, and R^{5e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)R^{a}, —C(O)N(R^{a})S(O)R^{a}, —S(O)OR^{a}, —S(O)N(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

Embodiment 9. The compound of Embodiment 8, a pharmaceutically acceptable salt, solvate, hydrate, cocrystal, or prodrug thereof, wherein the compound has a structure according to formula (10):

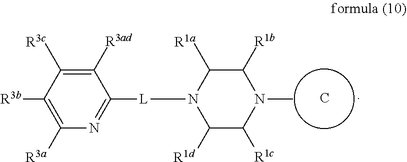

Embodiment 10. The compound of Embodiment 8, a pharmaceutically acceptable salt, solvate, hydrate, cocrystal, or prodrug thereof, wherein the compound has a structure according to formula (11):

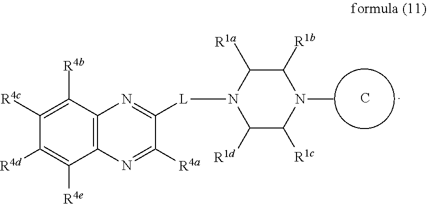

Embodiment 11. The compound of Embodiment 9 or 10, wherein R^{1a}, R^{1b}, and R^{1d} are each H.

Embodiment 12. The compound of any one of Embodiments 9-11, wherein R^{1c} is substituted C₁₋₆ alkyl, optionally substituted ethyl, optionally —(CH₂)₂—OH.

Embodiment 13. The compound of any one of Embodiments 9, 11, or 12, wherein R^{3a}, R^{3b}, R^{3c}, and R^{3d} are each H.

Embodiment 14. The compound of any one of Embodiments 10-12, wherein R^{4b}, R^{4c}, R^{4d}, and R^{4e} are each H.

Embodiment 15. The compound of any one of Embodiments 10-12 or 14, wherein R^{4a} is C₁₋₆ alkyl, optionally —CH₃.

Embodiment 16. The compound of any one of Embodiments 1-6 or 8-15, wherein C is selected from:

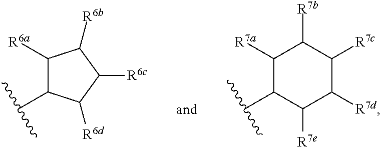

wherein R^{6a}, R^{6b}, R^{6c}, R^{6d}, R^{7a}, R^{7b}, R^{7c}, R^{7d}, and R^{7e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)R^{a}, —C(O)N(R^{a})S(O)R^{a}, —S(O)OR^{a}, —S(O)N(R^{a})R^{b}, —S(O)N(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

Embodiment 17. The compound of Embodiment 16, wherein C is:

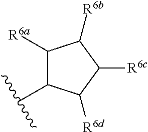

and
R^{6a}, R^{6b}, R^{6c}, and R^{6d} are each H.

Embodiment 18. The compound of Embodiment 16, wherein C is:

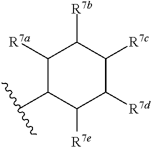

and
R^{7a}, R^{7b}, R^{7c}, R^{7d}, and R^{7e} are each H.

Embodiment 19. The compound of any one of Embodiments 1-18, wherein L is —(CH₂)₁₋₆—, optionally L is —(CH₂)—.

Embodiment 20. The compound of any one of Embodiments 2-5, 7, or 8, or a pharmaceutically acceptable salt, solvate, hydrate, cocrystal, or prodrug thereof, wherein the compound has a structure according to formula (3):

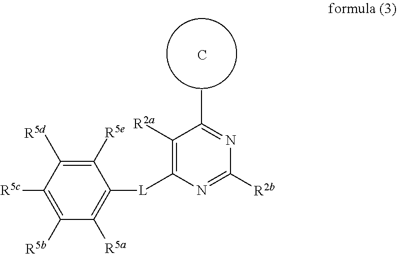

wherein in formula (3):
   B is an optionally substituted monocyclic aryl; and
      C is an optionally heteroaryl

Embodiment 21. The compound of Embodiment 20, wherein R^{2a} and R^{2b} are each H.

Embodiment 22. The compound of Embodiment 20 or 21, wherein R^{5b}, R^{5c}, R^{5d}, and R^{5e} are each H.

Embodiment 23. The compound of any one of Embodiments 20-22, wherein R^{5a} is —OR^{a}, optionally —OH.

Embodiment 24. The compound of any one of Embodiments 20-23, wherein C is:

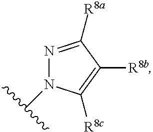

wherein R^{8a}, R^{8b}, and R^{8c} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{a}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

Embodiment 25. The compound of Embodiment 24, wherein R^{8a} and R^{8c} are each H.

Embodiment 26. The compound of Embodiment 24 or 25, wherein R^{8b} is C₁₋₆ alkyl, optionally —CH₃.

Embodiment 27. The compound of any one of Embodiments 1-26, wherein L is —CR^{a}═N—NR^{a}—, optionally L is —CH═N—NH—.

Embodiment 28. The compound of any one of Embodiments 1-27, wherein the compound is an allosteric activator of the β₂-adrenoceptor.

Embodiment 29. The compound of any one of Embodiments 1-27, wherein the compound is an allosteric inhibitor of the β₂-adrenoceptor.

Embodiment 30. The compound of any one of Embodiments 1-29, wherein the compound is not a compound of any one of formula 1001-1008.

Embodiment 31. A pharmaceutical composition comprising a compound of any one of Embodiments 1-30, or a pharmaceutically acceptable salt, solvate, hydrate, cocrystal, or prodrug thereof, and a physiologically compatible carrier medium.

Embodiment 32. A method of treating a disease or disorder alleviated by allosterically modulating β₂-adrenoceptor in a patient in need thereof, the method comprising administering to the patient a therapeutically effective amount of a compound of any one of Embodiments 1-29, or a pharmaceutically acceptable salt, solvate, hydrate, cocrystal, or prodrug thereof.

Embodiment 33. A method of treating a disease or disorder alleviated by allosterically modulating β₂-adrenoceptor in a patient in need thereof, the method comprising administering to the patient a therapeutically effective amount of a pharmaceutical composition of Embodiment 30, or a pharmaceutically acceptable salt, solvate, hydrate, cocrystal, or prodrug thereof.

Embodiment 34. The method of Embodiment 32 or 33, wherein the method further comprises allosterically activating β₂-adrenoceptor.

Embodiment 35. The method of any one of Embodiments 32-34, wherein the disease or disorder is an inflammatory disease.

Embodiment 36. The method of Embodiment 35, wherein the inflammatory disease is selected from the group consisting of chronic obstructive pulmonary disease (COPD), asthma, acute respiratory distress syndrome (ARDS), and acute lung injury (ALI), optionally wherein the inflammatory disease is asthma or COPD.

Embodiment 37. The method of any one of Embodiments 32-34, wherein the disease or disorder is a cardiovascular disease.

Embodiment 38. The method of Embodiment 37, wherein the cardiovascular disease is selected from the group consisting of cardiac arrhythmia, cardiomyopathy, myocardial infarction, heart valve disease, and heart failure, optionally wherein the cardiovascular disease is heart failure.

Embodiment 39. The method of any one of Embodiments 32-38, wherein the compound is of formula 1001, or a pharmaceutically acceptable salt, solvate, hydrate, cocrystal, or prodrug thereof.

Embodiment 40. The method of any one of Embodiments 32-38, wherein the compound is of formula 1002, or a pharmaceutically acceptable salt, solvate, hydrate, cocrystal, or prodrug thereof.

Embodiment 41. The method of any one of Embodiments 32-38, wherein the compound is of formula 1003, or a pharmaceutically acceptable salt, solvate, hydrate, cocrystal, or prodrug thereof.

Embodiment 42. The method of any one of Embodiments 32-41, wherein the compound, or pharmaceutically acceptable salt, solvate, hydrate, cocrystal, or prodrug thereof, is administered in a dosage unit form.

Embodiment 43. The method of Embodiment 42, wherein the dosage unit form comprises a physiologically compatible carrier medium.

The following examples describe the disclosure in further detail. These examples are provided for illustrative purposes only, and should in no way be considered as limiting the disclosure.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: In Silico Identification of a β₂-Adrenoceptor Allosteric Site that Selectively Augments Canonical β₂AR-Gs Signaling and Function

The following example describes the identification of an allosteric site on β₂AR that can modulate the activity of β-agonists. The Site Identification by Ligand Competitive Saturation (SILCS) computational method was employed to comprehensively map the entire 3D structure of in silico-generated β₂AR intermediate conformations and identified a putative allosteric binding site. Subsequent database screening using SILCS identified drug-like molecules with the potential to bind to the site. Experimental assays in HEK293 cells (expressing recombinant wild-type human β₂AR) and human ASM cells (expressing endogenous β₂AR) identified positive and negative allosteric modulators (PAMs and NAMs) of β₂AR, as assessed by regulation of β-agonist-stimulation of cyclic AMP generation. PAMs/NAMs had no effect on β-agonist-induced recruitment of β-arrestin to β₂AR or β-agonist-induced loss of cell surface expression in HEK293 cells expressing β₂AR. Mutagenesis analysis of β₂AR confirmed the SILCS identified site based on mutants of amino acids R131, Y219, and F282. Finally, functional studies revealed augmentation of D-agonist-induced relaxation of contracted human ASM cells and bronchodilation of contracted airways. These findings identify a novel allosteric binding site on the β₂AR, the activation of which selectively augments β-agonist-induced Gs signaling, and increases relaxation of ASM cells, the principal therapeutic effect of β-agonists.

Signaling studies of the SILCS-selected compounds identified five positive (PAM) and three negative (NAM) allosteric modulators (AMs) of the β₂AR based on their capacity to modulate isoproterenol (ISO)-induced cAMP generation. Interestingly, none of the AMs affected either ISO-induced β-arrestin recruitment to the β₂AR or β₂AR internalization. Computational and mutagenesis studies revealed the requirement of the β₂AR amino acids R131, Y219, and F282 for binding of the AMs. In addition, the PAMs and NAMs identified were able to modulate ISO-mediated relaxation of human ASM cells and bronchodilation of airways in human and murine precision-cut lung slices. This Example describes the identification of an allosteric site on the β₂AR and characterization of β₂AR AMs that bind to that site capable of selectively enhancing canonical Gs signaling induced by β-agonists in human ASM cells to improve the therapeutic function of the β₂AR.

### 1. Results

### 1.1. Molecular Dynamics Simulations to Identify Intermediate Conformational States.

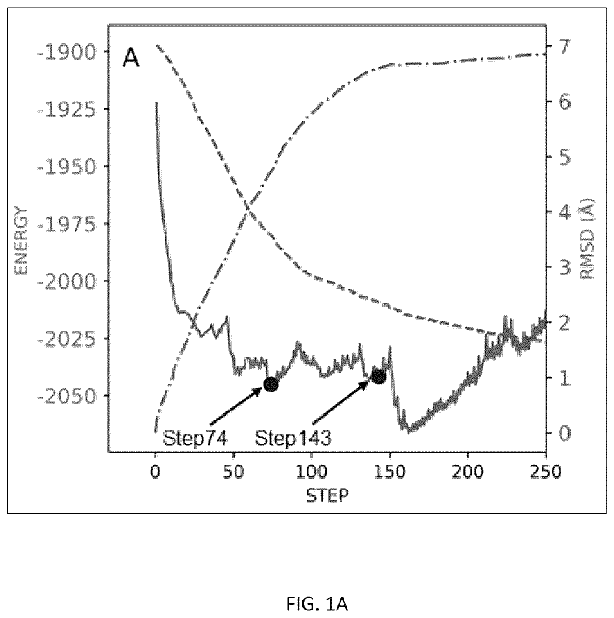

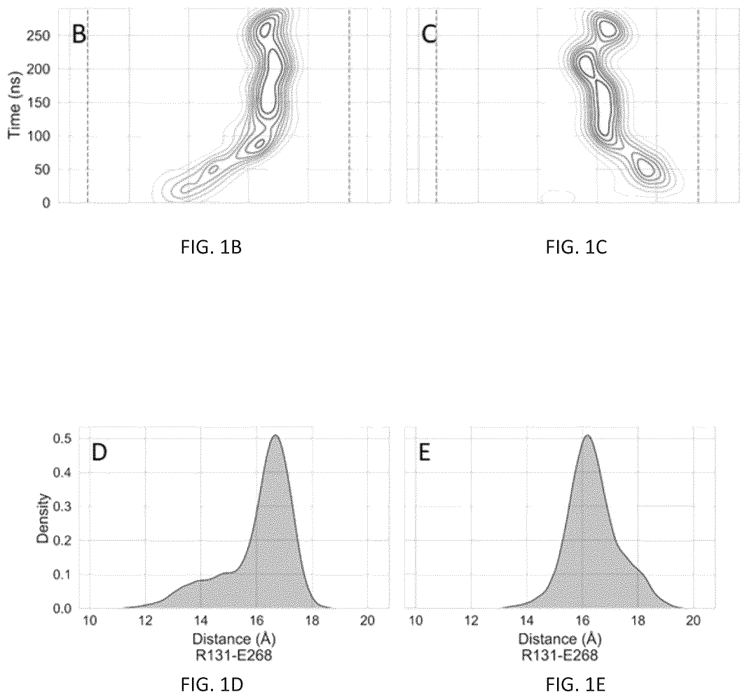

Molecular modeling and dynamics were applied to identify conformations of the β₂AR intermediate to those of the active and inactive forms of the receptor. This was performed using the protein structure morphing program Climber which identifies conformations between two input conformations. Starting from the active conformation, the structure was gradually morphed leading to a decrease in the Climber energy and the root-mean square difference (RMSD) with respect to the inactive conformation. The RMSD increased with respect to the active form (FIG. 1A). At the global minimum of the Climber energy, reached at approximately morphing step 160, the receptor attains the inactive conformation with respect to the transmembrane helices with the subsequent increase in Climber energy associated with the final morphing of loops and side chain atoms. Two conformations, steps 74 and 143, corresponding to energy minima along the morphing trajectory with conformations intermediate to the active and inactive were selected (FIG. 1A).

These two conformations were then subjected to a series of MD simulations to comprehensively sample conformational space of the receptor. The simulations were performed on the apo, agonist bound, and inverse agonist bound forms of both intermediates, with three individual simulations of each form performed for each of the two intermediates. The ionic lock distance between residues R131 and E268 was used to define intermediate conformations with respect to the known inactive and active structures. However, if the ionic lock formed any salt bridge interactions during the simulations, those simulations were not considered further.

Analysis of the sampling of the β₂AR conformations in the MD simulations based on the ionic lock distance is shown in FIGS. 1B-1E. The results shown in FIGS. 1B-1E are based on simulations initiated from step 74, where the ionic lock is initially in an inactive-like conformation with an ionic lock distance of 13.2 Å at the beginning of the MD simulations, compared to the distance of 11 Å present in the inactive 5X7D crystal structure. In the simulation initiated with the agonist BI-167107 the distance is steadily progressing towards a more active-like conformation with an average distance of ˜16 Å sampled towards the end of the simulation (FIG. 1B). In the simulation initiated with carazolol, which initially assumes an ionic lock distance of ˜14.5 Å, the system undergoes a large shift in conformation at approximately 25 ns (FIG. 1C) sampling ionic lock distances around 18 Å, similar to the distance of 19 Å observed in active-form crystal structure 3SN6. The associated conformations represent intermediate states with the average ionic-lock distance intermediate to the experimentally observed distances from the crystal structures indicated in FIGS. 1B and C. Subsequently, representative structures from the MD simulations were selected using RMSD clustering, for identifying conformations in which the ionic lock distance corresponded to the average value from the MD simulations (FIGS. 1D and 1E). These structures were from simulations initiated from Climber step 74 individually initiated with BI-167107 and carazolol, which are referred to as Step74A and Step74B respectively.

### 1.2. SILCS Allosteric Binding Site Identification.

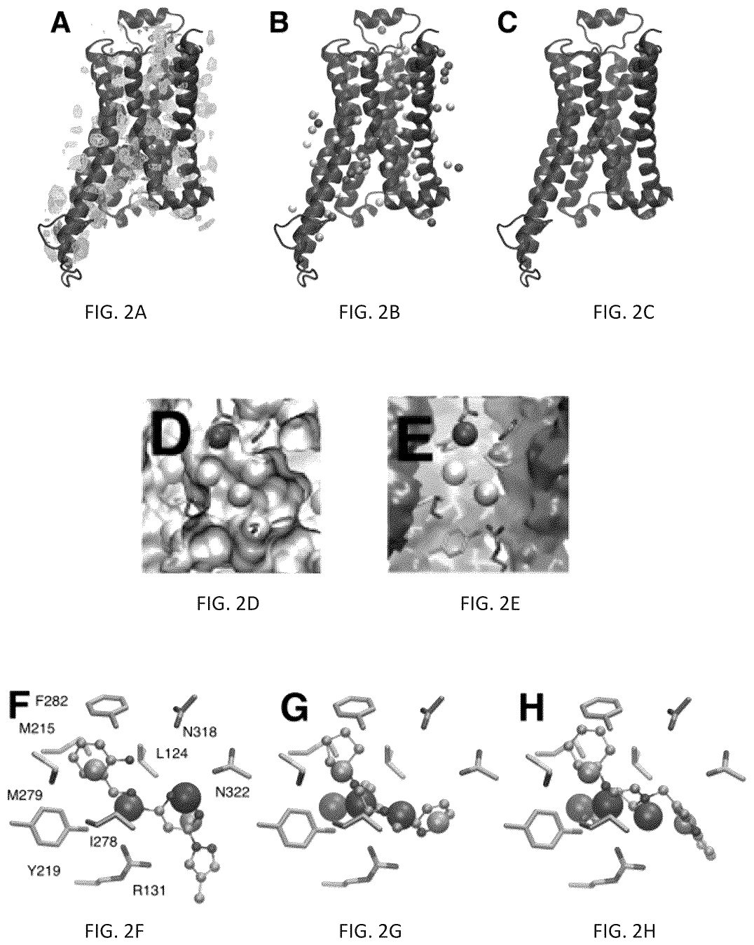

Identification of possible allosteric binding sites and subsequent database screening to identify putative novel AMs were performed using SILCS methodology. SILCS simulations were performed to generate functional group affinity maps (referred to as FragMaps) for the intermediate β₂AR conformations, Step74A and Step74B. The SILCS method is based on Grand Canonical Monte Carlo (GCMC) MD simulations using an explicit aqueous environment that includes 8 solutes. During the SILCS simulations, water and solutes, representative of different functional groups, compete around and throughout the entire protein, including cryptic pockets that are deep or totally inaccessible to the aqueous environment. Shown in FIG. 2A are the SILCS FragMaps overlaid on the Step74A conformation used to initiate the SILCS simulation. Evident are the apolar FragMaps around the transmembrane (TM) region of the protein that occupy regions adjacent to the hydrophobic region of the bilayer, negative charged maps on the intracellular face of the receptors, particularly around the ends of TM helices 5 and 6, and the presence of apolar, hydrogen bond donor and acceptor, and positive FragMaps throughout the interior core of the receptor. These latter maps indicate the utility of the SILCS approach for identifying interior regions on the protein amenable to the binding of various functional groups. Identification of putative allosteric binding sites was done using the SILCS-Hotspots approach. This involved comprehensive docking of 101 mono- and bicyclic chemical fragments in the field of the FragMaps to generate fragment binding poses that encompass the entire protein. Two rounds of clustering were then performed from which final Hotspots that act as the basis for allosteric site identification were identified. Shown in FIG. 2B are the Hotspots on the Step74A conformer. The presence of Hotspots throughout the protein, including the surface and interior regions, is evident, consistent with the distribution of the FragMaps. Further in-depth visual inspection of the Hotspots was then undertaken to identify possible allosteric sites to which drug-like molecules would bind, potentially acting as AMs.

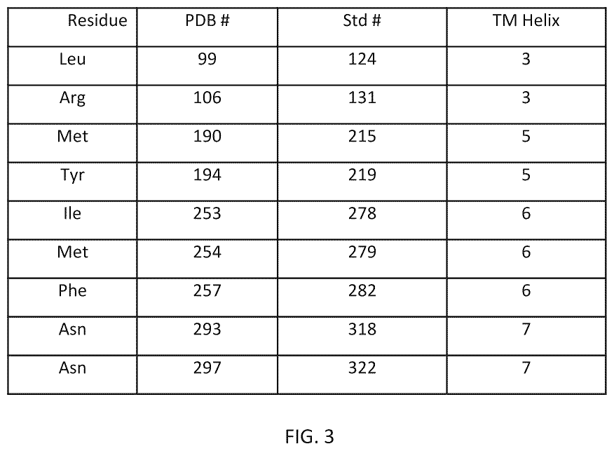

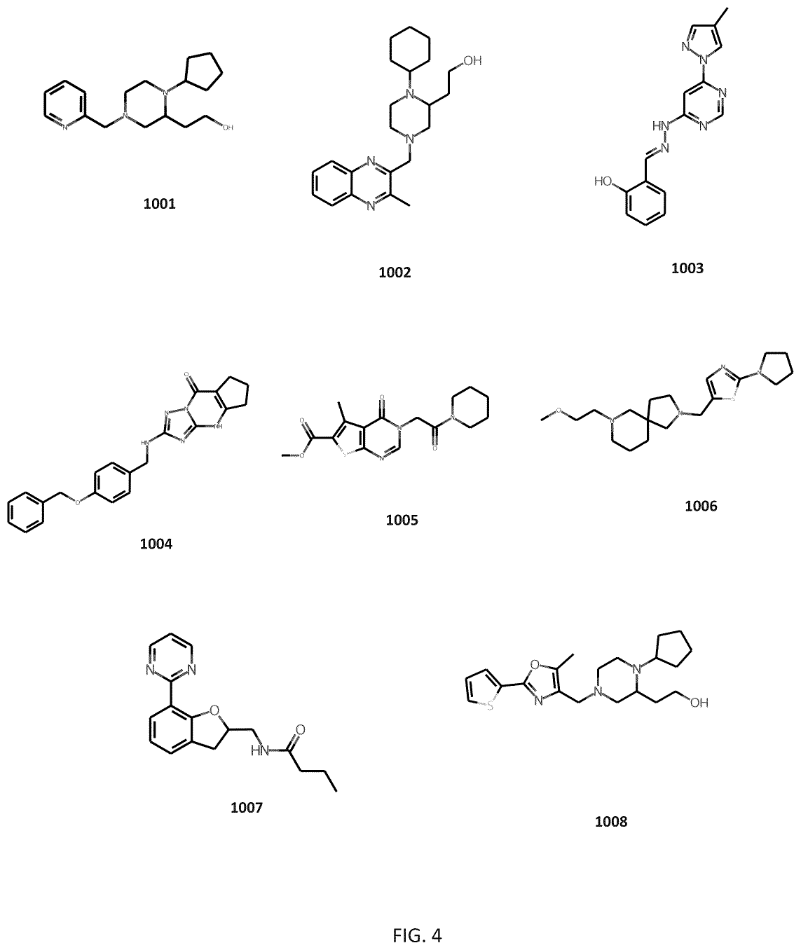

Allosteric site selection focused on the identification of collections of adjacent Hotspots in which fragments occupying those sites could potentially be linked to create drug-like molecules. In this process emphasis is on the relative location of adjacent Hotspots and the presence of open regions between those hotspots based on SILCS exclusion maps, with the rank ordering of the Hotspots given lower priority, as described previously. In addition, sites located in the vicinity of the residues known to impact the activity of β₂AR were given priority. From this process, a site comprised of 3 Hotspots was identified. The site is present in both the Step74A and Step74B systems between helices 3, 5, 6 and 7 (FIG. 2C, FIG. 3). While analysis of the solvent accessible surface in that region (FIG. 2D) shows minimal free space in the vicinity of those Hotspots as well as indicating the location of the binding site on the interior of the TM bundle of helices, analysis of the SILCS exclusion maps around the Hotspots (FIG. 2E) shows that the region between and around the Hotspots is accessible for accommodating larger ligands. The exclusion maps are defined based on the regions of the proteins in which the water or solutes do not sample during the SILCS simulations and, therefore, represent the extent to which the protein can relax to allow ligand binding in contrast to a simple solvent accessible surface based on a single protein structure. Accounting for protein flexibility in the SILCS method clearly identifies regions that can become accessible thereby facilitating the identification of putative binding sites not evident in single modelled or experimental structures.

### 1.3. Allosteric Modulator Identification.

In silico database screening to identify putative AMs that bind to the identified site initially involved screening using pharmacophores generated with the SILCS-PHARM approach. This approach involves generation of pharmacophore features in the vicinity of the selected Hotspots based on the FragMaps in combination with clustering of the voxels that define the different FragMaps. From these pharmacophore features, 8 pharmacophore hypotheses were selected for database screening, with each containing 4 or 5 pharmacophore features.

Two example pharmacophores that ultimately identified allosteric modulators are shown in FIGS. 2F and 2G and 2H, respectively, along with the amino acid sidechains lining the putative binding pocket (labeled in FIG. 2F and FIG. 3). The complementarity between the pharmacophore features and the adjacent amino acids is evident. For example, the right side of 2F shows a hydrophobic feature (cyan sphere) surrounded by two Met, a Phe, and a Leu residue. Interestingly, adjacent to R131 is a hydrogen bond acceptor feature indicating that despite the positive charge of R131 a negative functional group on a ligand in that region is not highly favored.

Pharmacophore screening using Pharmer produced up to 10,000 hits/pharmacophore based on RMSD of the ligand functional groups with respect to the pharmacophore features. The selected compounds were then pooled and subjected to local optimization and scoring using SILCS-MC pose refinement from which the Ligand Grid Free Energy (LGFE) scores were calculated. From this step, the top 1000 compounds were selected. These compounds were then subjected to chemical fingerprint clustering and bioavailability analysis using the 4D-Bioavailability metric from which a total of 100 chemically-diverse compounds were selected for purchase from the commercial vendor. All the 100 compounds were successfully obtained and subjected to biological evaluation yielding 8 lead compounds that modulated the activation of β₂AR (FIG. 4). Of these, 5 were PAMs and 3 were NAMs as described below. The predicted bound conformation of the 3 AMs subjected to comprehensive experimental validation are included in FIG. 2F, FIG. 2G and FIG. 2H.

1.4. Lead Compounds Modulate Agonist-Induced cAMP Generation.

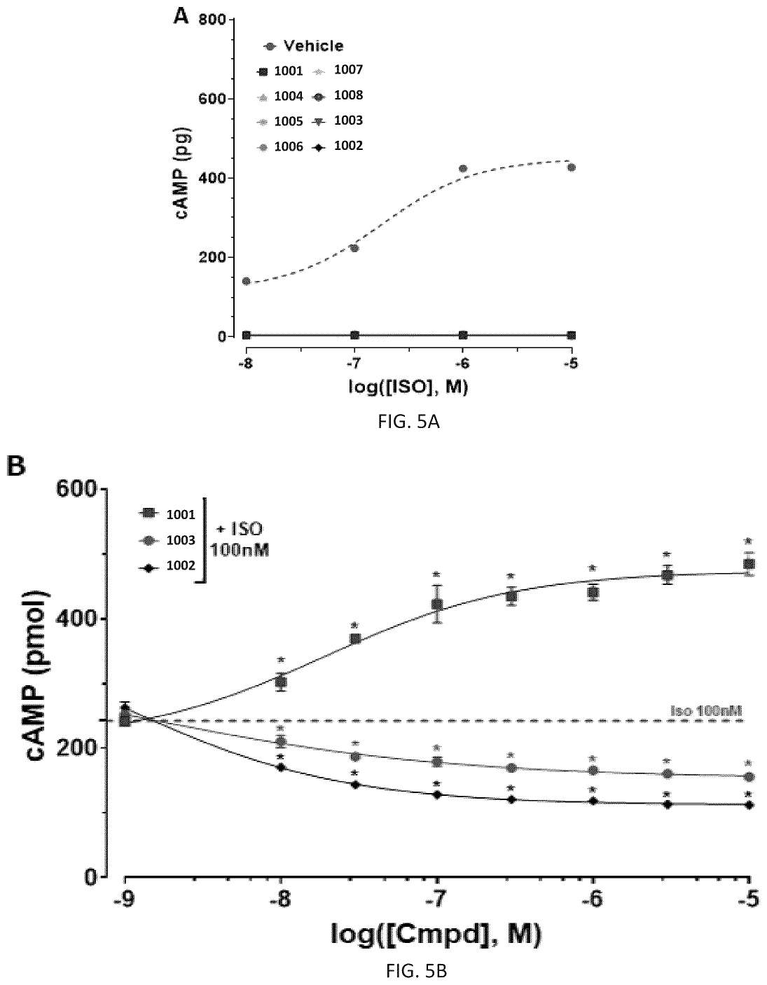

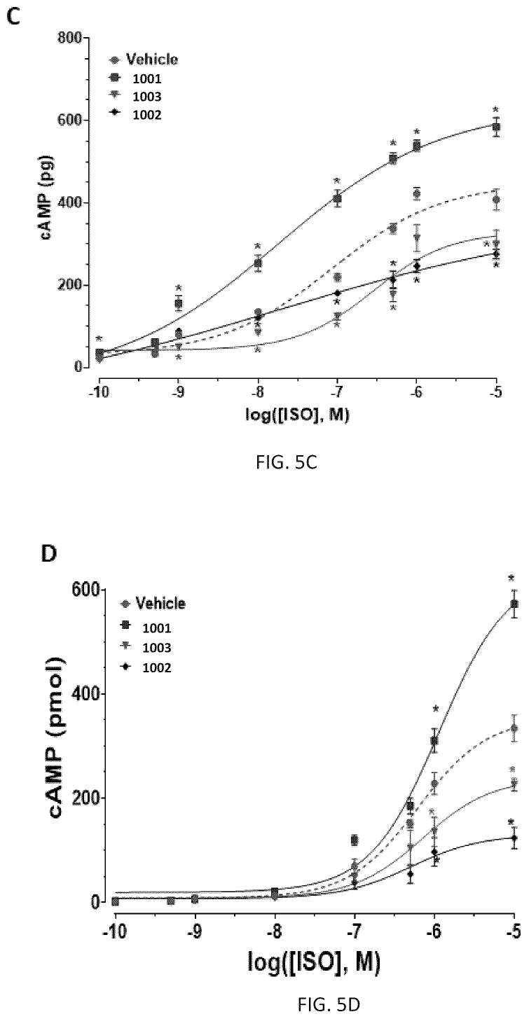

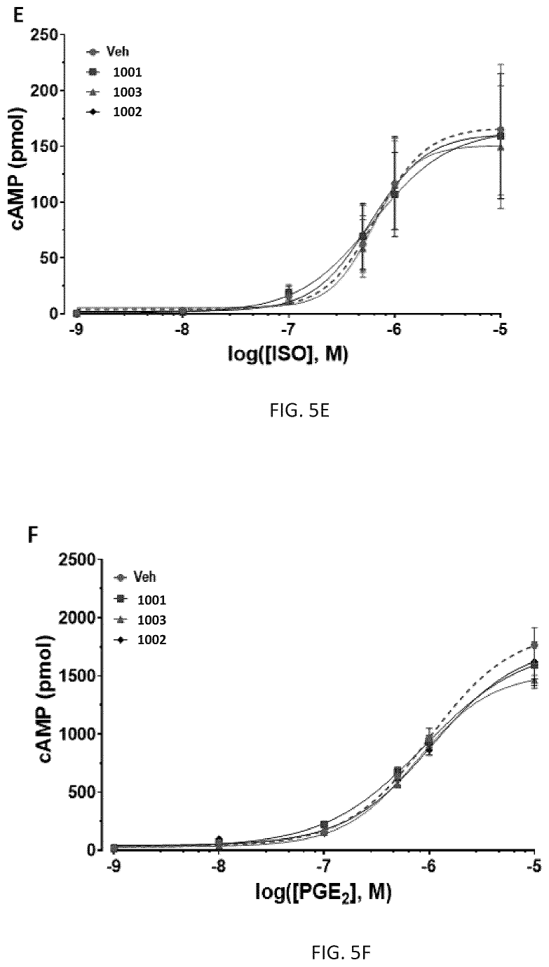

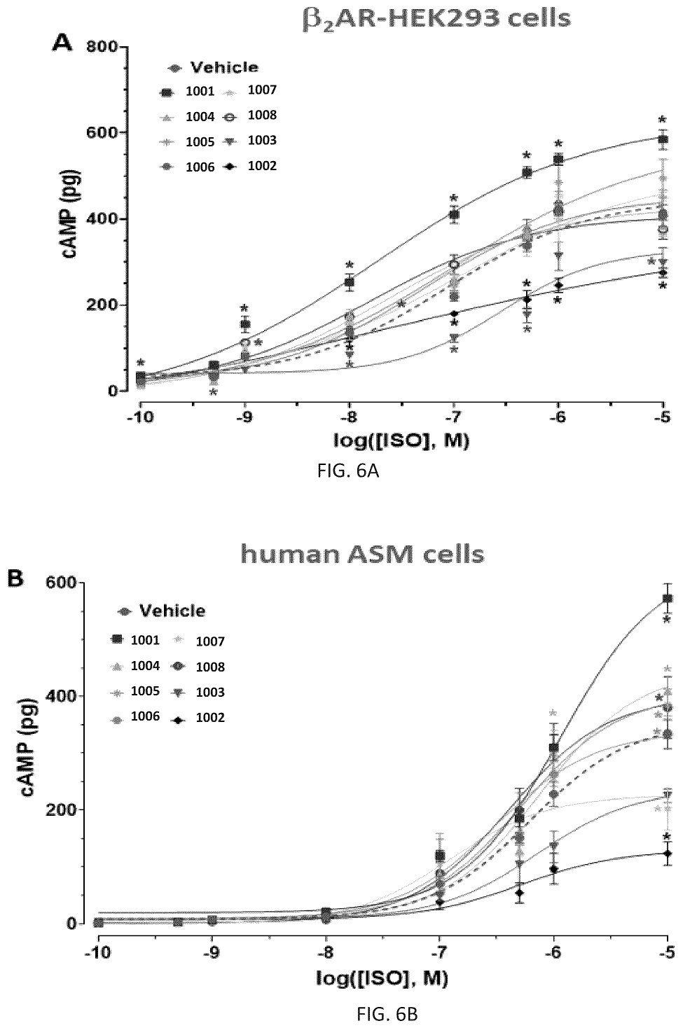

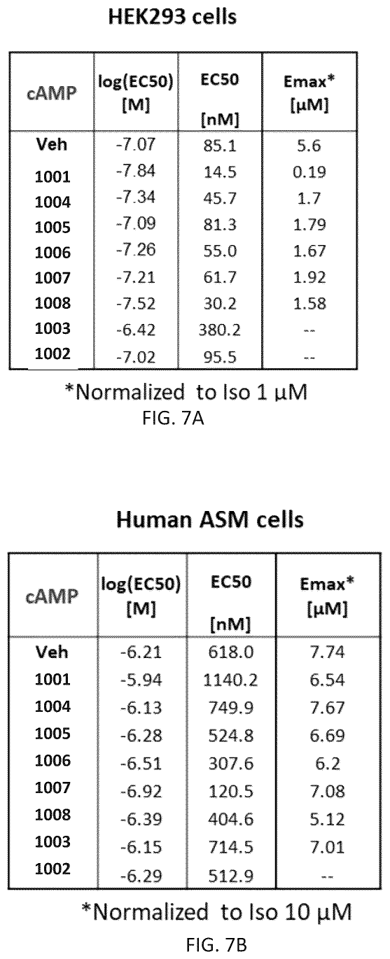

Activation of β₂AR by orthosteric ligands leads to activation of Gαs subunit, which in turn activates AC to hydrolyze ATP to produce cAMP. To determine the biological activity of the AMs of the β₂AR, HEK293 cells overexpressing human wild type β₂AR were stimulated with different concentrations of test compounds and evaluated for cAMP production using a cAMP ELISA. None of the compounds tested promoted cAMP accumulation in HEK293 cells (FIG. 5A). However, when cells were treated with varying concentration of test compounds along with 100 nM isoproterenol (ISO), the PAMs and NAMs modulated ISO-induced cAMP generation in a dose dependent manner (FIG. 5B). All subsequent studies were carried out using AMs at 100 nM based on these dose-response studies. Further, treatment of HEK293 cells with ISO increased cellular cAMP levels in a concentration-dependent manner (FIG. 5C). Co-stimulation with ISO and 100 nM of different AMs significantly increased or decreased the dose-dependent effect of ISO on cAMP accumulation (FIG. 5C and FIG. 6A). Biological evaluation of obtained compounds yielded 8 compounds that modulated the activation of β₂AR. Of these, compound 1001 was the most effective PAM and compounds 1002 and 1003 were the most effective NAMs (FIG. 5C and FIG. 6A). These compounds were used in subsequent studies. EC₅₀ and Eₘₐₓ values of ISO alone or in combination with PAMs and NAMs are depicted in FIG. 7A.

To evaluate the observed modulating effects of PAMs and NAMs on β₂AR-Gs-cAMP signaling in a physiologically relevant system, the effect of the compounds in primary human ASM cells, which express β₂AR endogenously, was further tested. Concomitant treatment of human ASM cells with PAMs or NAMs enhanced or attenuated ISO-induced cAMP generation, respectively (FIG. 5D and FIG. 6B). EC₅₀ and Eₘₐₓ values of ISO-induced cAMP generation in human ASM cells are given in FIG. 7B.

### 1.5. AMs are Specific for β₂AR.

In order to establish the specificity of the AMs to β₂AR, the effect of AMs on ISO-induced cAMP generation in HEK293 cells expressing human β₁AR was tested. AMs did not modulate ISO-induced cAMP generation in HEK293 cells expressing β₁AR (FIG. 5E). Further, in human ASM cells, which express Gs-coupled EP2 and EP4 receptors, PAMs or NAMs did not modulate prostaglandin E2-induced cAMP generation (FIG. 5F). Collectively, these findings suggest the specificity of the AMs to β₂AR.

### 1.6. Lead Compounds do not Affect β-Agonist-Induced Recruitment of β-Arrestins.

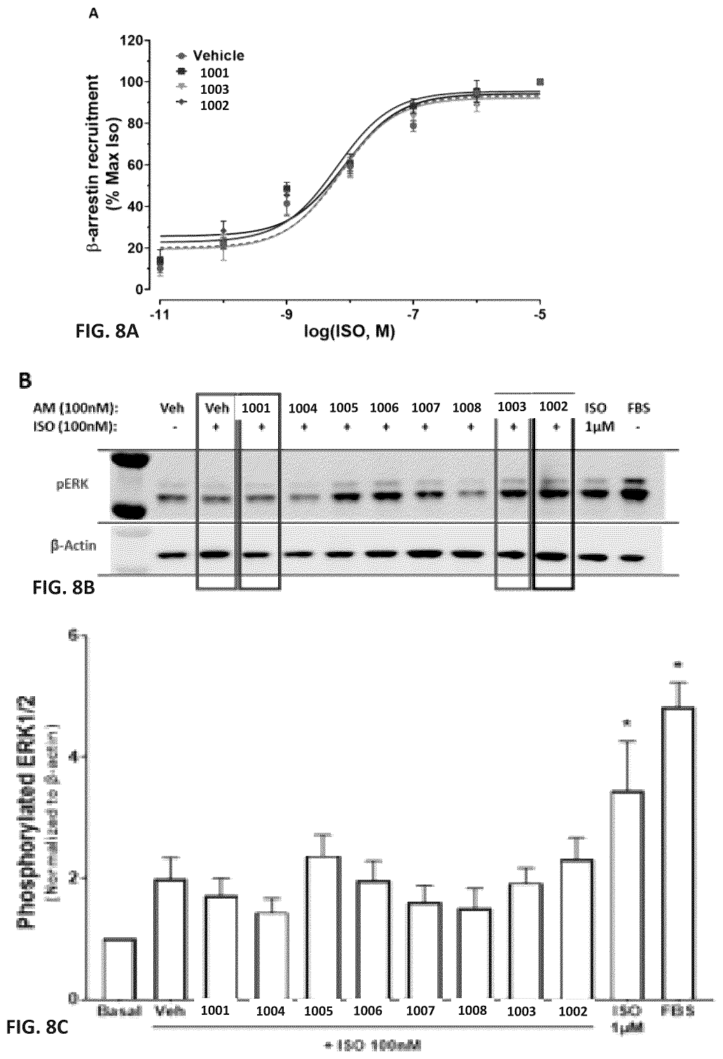

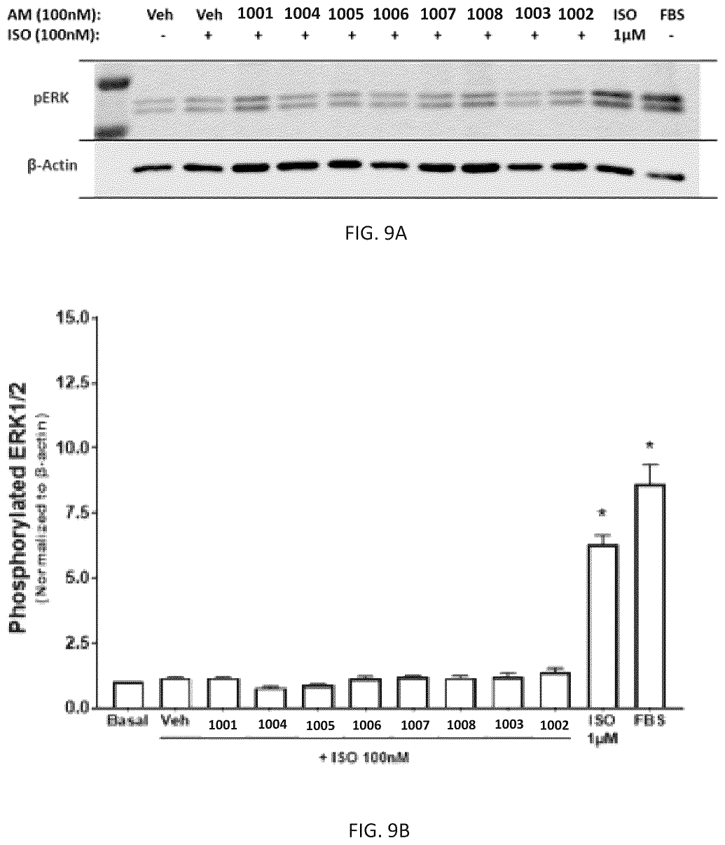

In addition to the canonical Gs signaling, the “balanced” b-agonists induce β-arrestin recruitment to the β₂AR resulting in β₂AR desensitization and activation of non-canonical signaling such as ERK1/2 signaling. Therefore, the aim was to determine the effect of compounds 1001, 1002, and 1003 on ISO-induced β-arrestin2 recruitment to the β₂AR using a well-established bioluminescence resonance energy transfer (BRET) assay. ISO treatment of HEK293 cells expressing β₂AR resulted in a dose-dependent increase in recruitment of β-arrestin2 to the receptor (FIG. 8A). Concomitant treatment of cells with either PAM or NAMs did not alter ISO-mediated changes in BRET (FIG. 8A). Furthermore, the effect of AMs on ISO-induced phosphorylation of ERK1/2 was tested. Neither PAM nor NAMs altered ERK1/2 phosphorylation in HEK293 (FIG. 8B and FIG. 8C) or ASM cells (FIG. 9A and FIG. 9B). These data suggest that increased β₂AR-Gs signaling effected by the small molecule PAM occurs without alterations in β-arrestin recruitment to the β₂AR, or non-canonical β-arrestin-dependent signaling induced by orthosteric β-agonist.

### 1.7. AMs do not Modulate Binding of Orthosteric Agonist to β₂AR.

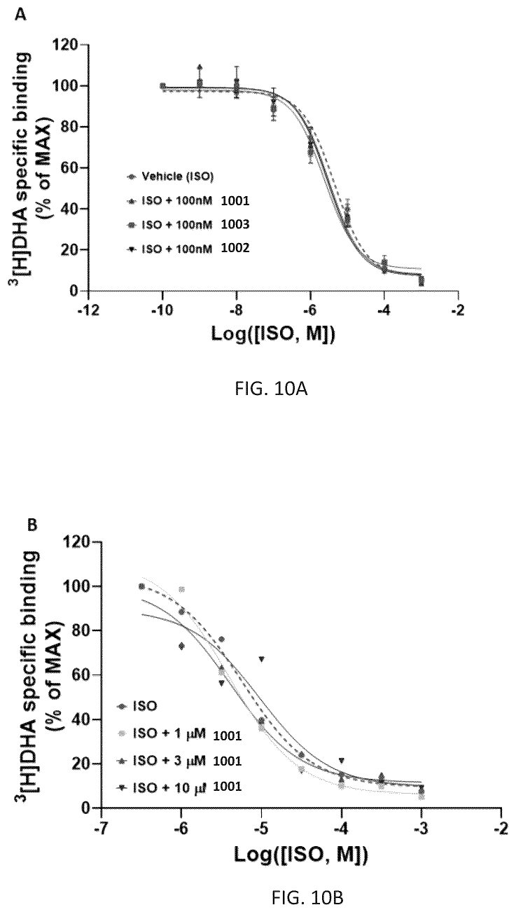

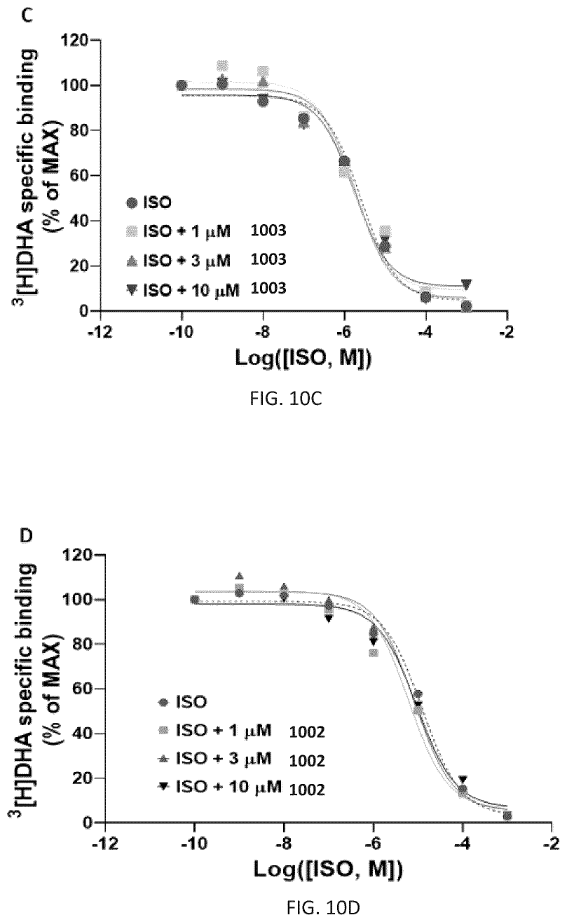

PAMs or NAMs have the potential influence β₂AR signaling by altering the affinity of the orthosteric ligand to β₂AR. Therefore, the effect of AMs on the binding affinity of ISO to β₂AR was tested using a well-established radiolabel ligand binding assay. AMs did not alter binding affinity of ISO to the β₂AR (FIGS. 10A-D).

### 1.8. Mutagenesis Studies Confirm the Mechanism of Action of Lead Compounds.

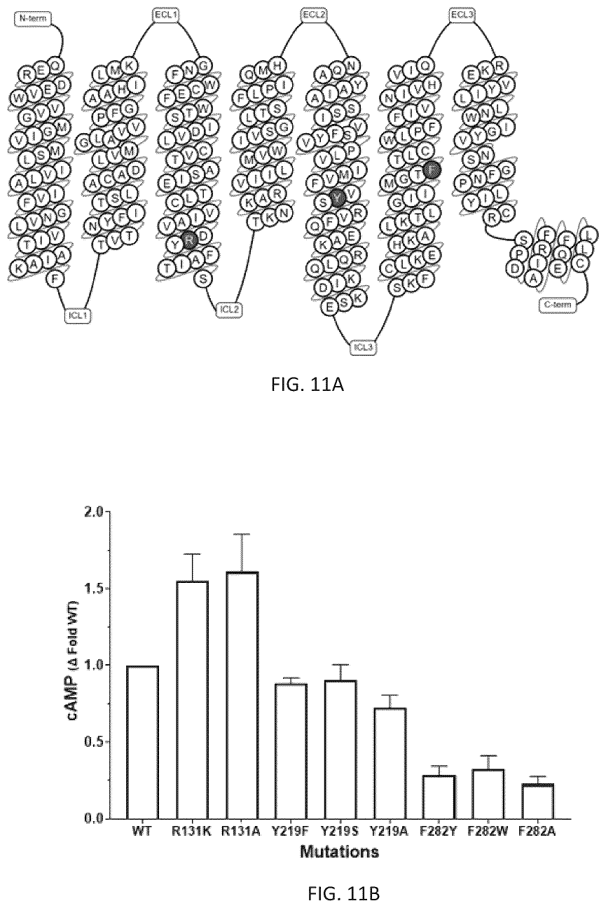

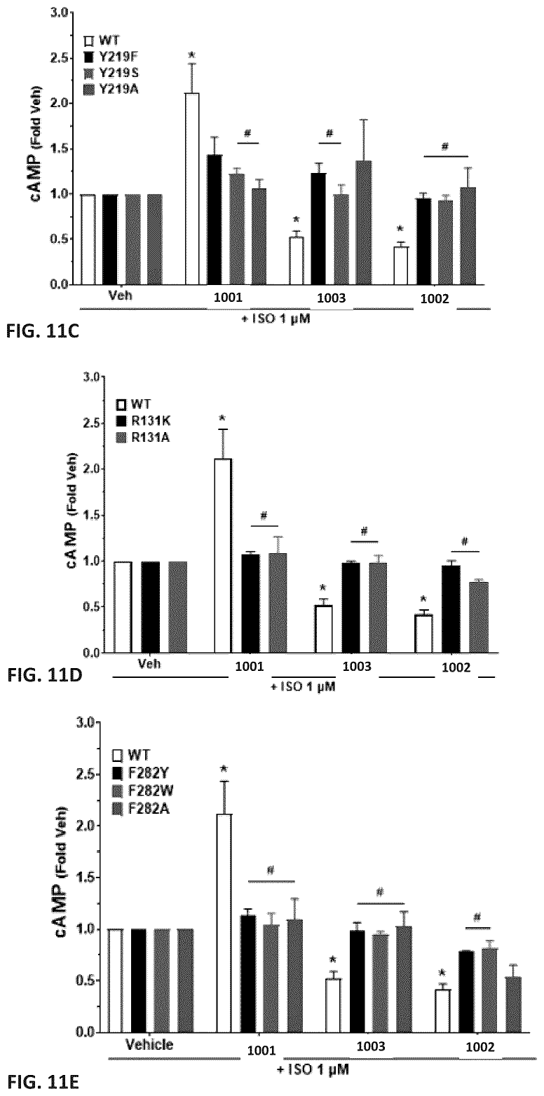

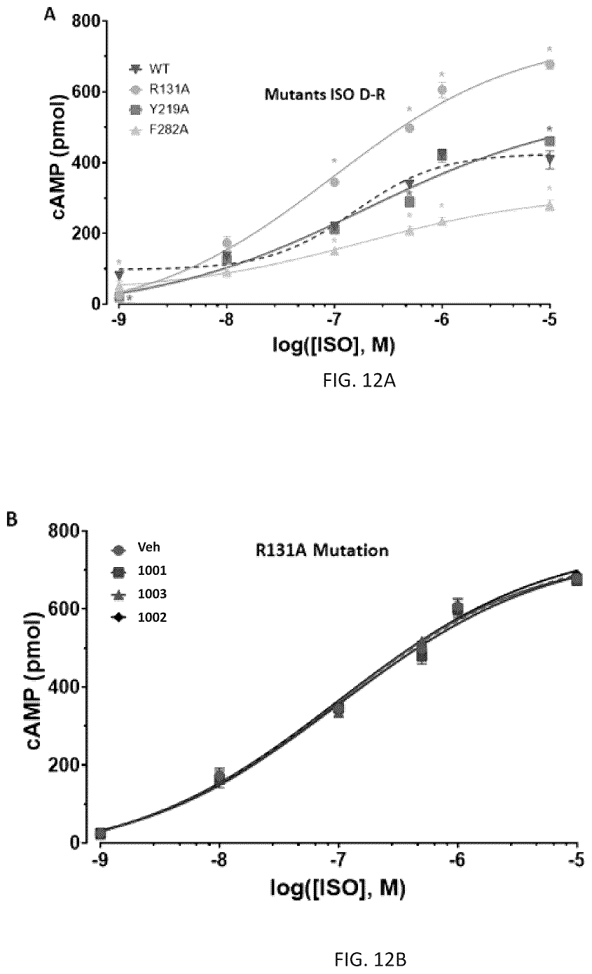

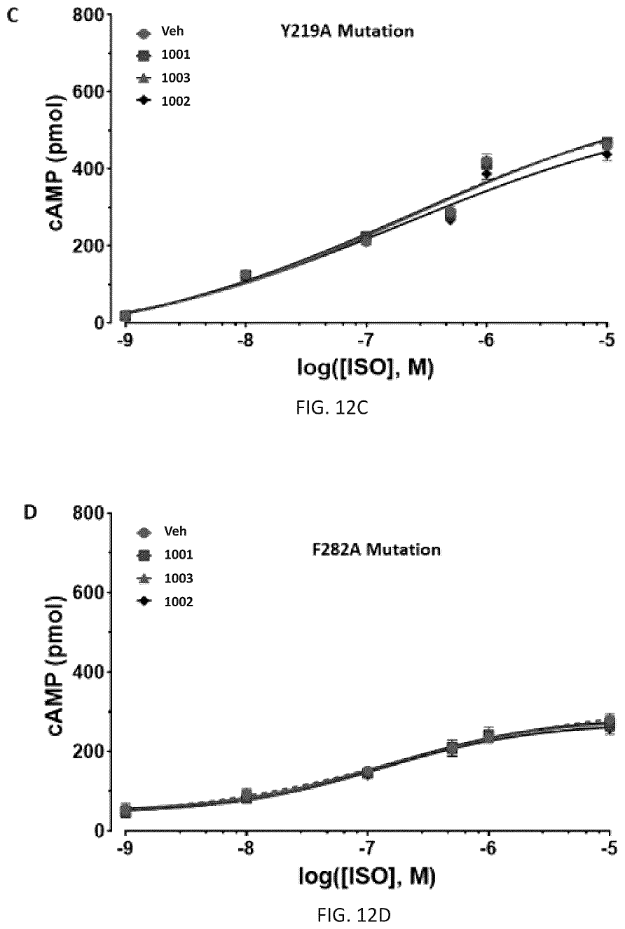

To validate the identity of the computationally identified allosteric site, β₂AR containing R131K, R131A, Y219F, Y219S, Y219A, F282Y, F282W, and F282A mutations was generated (FIG. 11A). Wild-type and mutant β₂AR were expressed in HEK293 cells, and ISO-induced cAMP generation was measured in the presence or absence of the PAM compound 1001, or NAM compounds 1002 and 1003 (FIGS. 11B-E). The compounds were also tested at different concentrations of ISO in the cells expressing mutant β₂AR (FIGS. 12A-D). The cAMP response was enhanced in cells expressing R131A and diminished in F282A mutant β₂AR compared to cells expressing wild-type β₂AR. The ISO-induced cAMP generation in cells expressing wild-type and Y219A mutant β₂AR was comparable (FIG. 12A). Further, as expected, compound 1001 enhanced ISO-induced cAMP generation in cells expressing wild-type β₂AR, and this effect was abolished in cells expressing mutant β₂AR (FIGS. 11C-E, FIGS. 12B-D). Furthermore, NAM compounds 1002 and 1003 inhibited ISO-induced cAMP generation in wild-type β₂AR expressing cells, and this inhibition was attenuated in cells expressing R131K, R131A, Y219F, Y219S, Y219A, F282Y, F282W, and F282A mutant β₂ARs (FIGS. 11C-E, FIGS. 12B-D). Collectively, these findings demonstrate the critical requirement of amino acids R131, Y219, and F282 of the β₂AR in forming the binding pocket of PAMs and NAMs.

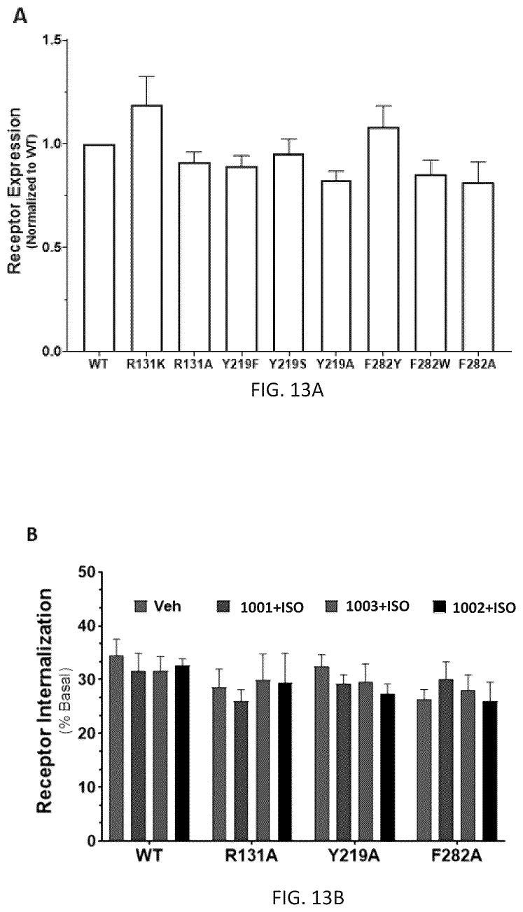

Mutagenesis of the β₂AR may influence the expression and translocation of the β₂AR to the cell surface. Therefore, to further validate the mutagenesis experiments, the cell surface expression of HA-tagged wild-type and mutant β₂AR using cell surface ELISA was assessed, as described previously. Baseline cell surface expression of different mutants of the β₂AR was similar to that of wild-type β₂AR (FIGS. 13A-B).

### 1.9. Lead Compounds do not Alter β-Agonist-Induced Loss of β₂AR Cell Surface Expression.

Previous studies have shown that stimulation of β₂AR with orthosteric ligands results in concentration- and time-dependent loss of cell surface expression of β₂AR. Therefore, the effect of the PAM or NAMs on the β-agonist-mediated loss of cell surface expression of the β₂AR was tested next. Treatment of HEK293 cells with ISO (100 nM) for 15 min resulted in a loss of cell surface expression of the β₂AR (FIG. 13B). Treatment of cells with PAM 1001 or NAMs 1002 and 1003 in combination with ISO did not affect the ISO-induced loss of cell surface expression of wild-type or mutant β₂AR (FIG. 13B).

### 1.10. Lead Compounds Augment β-Agonist-Induced ASM Relaxation.

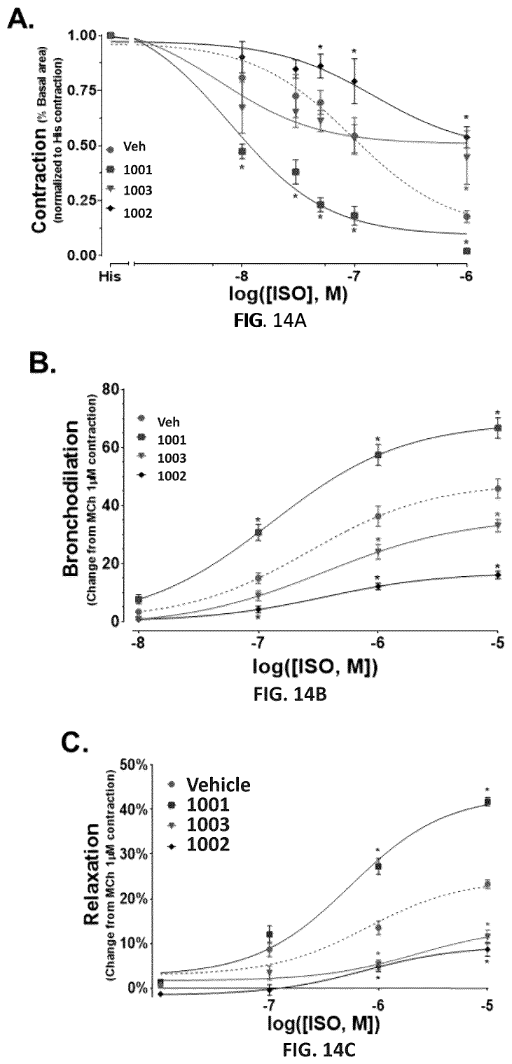

β-agonists are the drug of choice for management of acute bronchoconstriction in asthmatics, acting on the target ASM cells. The Gs-cAMP-protein kinase A signaling axis effected by β-agonists inhibits contraction of ASM cells via multiple mechanisms. Therefore, it was examined whether the modulation of this signaling pathway by the discovered PAM/NAMs translated into functional differences in β₂AR control of ASM contraction. In a standard gel-contraction assay, the collagen gels containing human ASM cells were incubated with different concentrations of ISO with vehicle or 100 nM AMs for 10 min, followed by stimulation with 10 μM histamine. The gel images were obtained before and after treatment with different agonists using an EVOS microscope, and a change in the gel area was calculated. Treatment with 10 μM histamine decreased the area of the gels, consistent with contraction of ASM cells. Pretreatment of gels with ISO inhibited the histamine-induced decrease in gel area (FIG. 14A). Pretreatment of gels with ISO in combination with compound 1001 significantly enhanced, whereas compounds 1002 and 1003 either slightly reduced or had no effect, on ISO-mediated inhibition of histamine-induced contraction of human ASM cells (FIG. 14A).

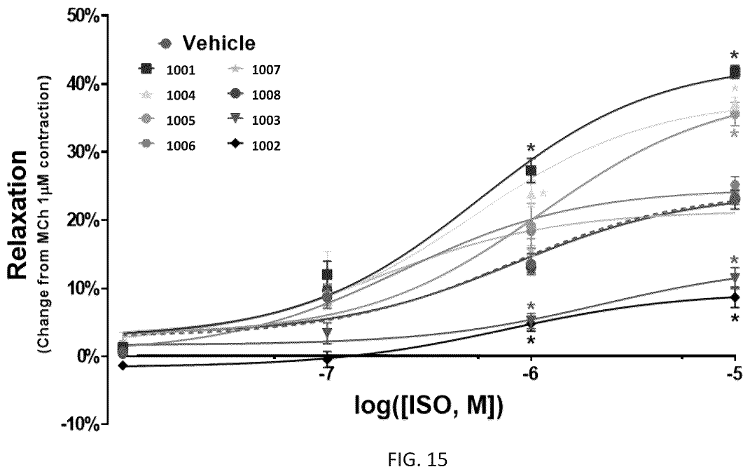

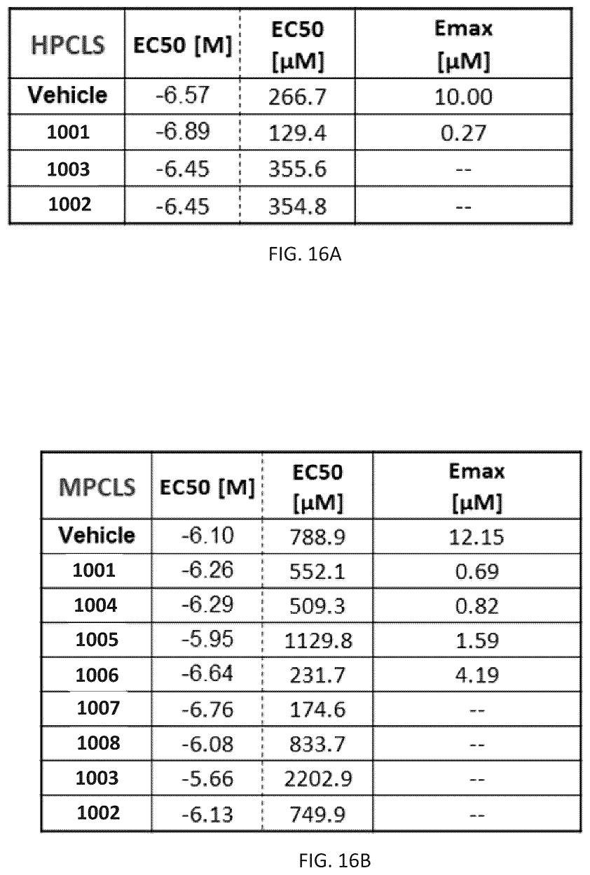

To further examine the effect of PAMs and NAMs on β-agonists-mediated regulation of bronchorelaxation in a physiologically-relevant model, human and murine precision-cut lung slices were used. Treatment of lung slices with the contractile agent methacholine resulted in the narrowing of airways. Lung slices were treated with increasing concentrations of ISO plus either vehicle or 100 nM of compounds 1001, 1002, and 1003, and a change in airway lumen area was determined. Treatment with ISO alone resulted in a dose-dependent increase in airway lumen area (bronchodilation). This effect was significantly enhanced in the presence of 100 nM compound 1001 and mitigated in the presence of compounds 1002 and 1003 (FIG. 14B, FIG. 14C, and FIG. 15); the other AMs tested had varying effect of ISO-induced bronchodilation in murine lung slices (FIG. 15). The EC₅₀ and Eₘₐₓ values for ISO-mediated relaxation of murine and human lung slices are presented in FIGS. 16A-B.

Collectively, these results from cell- and tissue-based models of ASM contraction strongly suggest that PAM properties of compound 1001 extend to $2AR-Gs function (bronchodilation), with the potential to be exploited therapeutically.

### 2. Discussion

Presented is a combined computational and experimental study to identify novel allosteric modulators of the β₂ AR, which offer the potential of improved therapeutic efficacy in the management of obstructive lung diseases such as asthma. The computational SILCS approach was used to identify previously unidentified putative ligand binding sites that would potentially act as allosteric sites. To facilitate the identification of such sites, intermediate conformations of β₂AR, with respect to the active and inactive conformations of the protein, were generated using the program Climber followed by MD simulations of the receptor in its apo form or in complex with BI-167107 or Carazolol. SILCS-Hotspots analysis on the selected conformations identified a putative allosteric binding pocket on the interior of the receptor, adjacent to the ionic lock, suitable for the binding of drug-like molecules. Subsequently, a combination of SILCS-Pharmacophore, Pharmer and SILCS-MC pose refinement programs were used to identify a collection of chemically diverse, commercially available compounds with suitable physiochemical properties as required for experimental studies. From this list, a total of 100 compounds were obtained and subjected to experimental evaluation. Among these, eight compounds modulated β-agonist-induced cAMP generation in HEK293 cells expressing β₂AR with no effect on the recruitment of β-arrestins to the β₂AR. In primary human ASM cells expressing endogenous β₂AR, the lead compounds enhanced β₂AR-mediated Gs signaling as measured by ISO-induced cAMP generation. Importantly, a lead PAM 1001 enhanced ISO-induced relaxation of human ASM cells and bronchodilation of human and murine airways ex vivo. These results demonstrate that PAM compound 1001 selectively augments canonical β₂AR-Gs-cAMP signaling that translates into superior β-agonist mediated bronchorelaxation. Although not wishing to be bound by any particular theory, these results suggest that AMs do not directly modulate the binding affinity of the orthosteric β-agonist to the receptor, or the AMs might function by stabilizing the intermediate β₂AR confirmations thus keeping the receptor active for longer.

### 2.1. Computational Approach to Allosteric Binding Site Identification.

SILCS is a co-solvent molecular simulation approach that maps the functional group affinity pattern of a protein, including the protein interior, taking into account protein flexibility as well as protein and functional group desolvation contributions. Notably, the SILCS-Hotspots method allows for docking a collection of fragments on the entire protein to identify putative binding sites suitable for drug-like molecules in a highly computational efficient fashion with a level of accuracy comparable to other state of the art computational methods such a free energy perturbation. The present results further represent the capability of the SILCS method to identify allosteric binding sites showing, notably, its utility against a GPCR. The technology takes advantage of the inclusion of protein flexibility in SILCS combined with the use of GCMC water and solute sampling to identify possible binding sites on the interior of a protein not evident in experimental crystallographic or cryo-EM structures. Further improvements in the SILCS approach may include enhancement of the conformational sampling of the protein, possibly through increasing the simulation temperature, as well as generating additional intermediate conformations of the GPCR. The latter could be achieved through a number of enhanced sampling simulation technologies.

These results demonstrate the identification of a previously unidentified allosteric site on the β₂AR comprised of amino acids R131, Y219, and F282. The SILCS Hotspots identified allosteric binding site (FIGS. 2B-2C) is located roughly 20 Å below the orthosteric binding site inside the transmembrane bundle. The Hotspots that make up the site were of interest as they are located just below the PIF connector motif (P211, 1121 and F282) and extend out towards the core of the TM bundle. This central site is completely inaccessible in the inactive conformation as it is occupied by TM helix 6. However, in the intermediate conformation identified using Climber and in the conformations sampled during the subsequent MD simulations, R131 shifts up towards the site previously occupied by helix 6 that has been observed in an experimental structure of the activated receptor. Although not wishing to be bound by any particular theory, these findings further suggest that mutation of R131 enhances ISO-induced cAMP accumulation (FIG. 12A) consistent with the previously published literature. Mutation in F282 on the contrary attenuated ISO-induced cAMP generation similar to what has been shown previously. The cAMP generation in cells expressing Y219A mutant receptor was comparable to the wild-type receptor. Importantly, the AMs did not have any effect on ISO-induced cAMP generation in cells expressing mutant β₂AR supporting the hypothesis that AMs potentially bind to a pocket formed by these three amino acids (FIGS. 11D-11E, FIGS. 12B-12C). Further, cell surface expression ELISA findings demonstrated that AMs do not modulate ISO-induced internalization of the β₂AR. A previous study demonstrated that mutation in Y219 renders the β₂AR resistant to agonist-induced desensitization and internalization. However, an ISO-induced loss of cell surface expression with the Y219 mutant was observed, most likely due to significant differences in experimental approach including different cell systems, which are known to produce variable results in studies of β₂AR regulation.

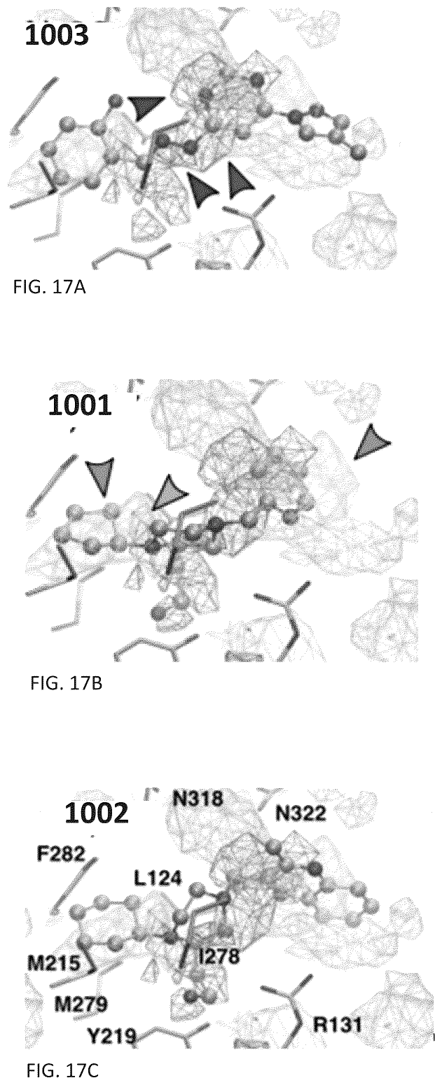

Notably, the present site does not correspond to a previously identified allosteric sites on β₂AR. The first identified β₂AR modulator, a NAM, binds to the intracellular termini of TM helices 1, 2, 6, and 7. A PAM (Cmpd 6), binds to a pocket formed by ICL2 and TM helices 3 and 4 and more recently, a NAM was identified and shown to bind to TM helices 3 and 5. Interestingly, these two allosteric sites are located on the protein-membrane interface and may potentially modulate the receptor response from the membrane side by impacting G-protein binding or by disrupting the polar network around the activation switch and thus stabilizing the inactive receptor conformation. Concerning the activity of the present allosteric modulators, the location of the site is below the orthosteric site in a deep pocket in the central region of the TM helices (FIGS. 2D-2E). Shown in FIGS. 17A-17C are bound orientations of 1001, 1002, and 1003 along with the SILCS FragMaps further demonstrating the compounds' ability to participate in favorable interactions with the binding site. The similar orientations of the compounds and location of the allosteric site along with the lack of large changes in the EC₅₀ values for ISO in the experiments assessing cAMP generation (FIGS. 5A-5F and FIGS. 7A-7B), indicate that the mechanism of action would be to stabilize specific conformations of the receptor rather than directly impacting the binding of agonists to the orthosteric site. This would involve a shift in the conformations toward more or less active conformations based on the details of the specific interactions of the compounds with the region around the binding site given that both PAMs and NAMs have been identified. In addition, these changes would impact G protein interactions over those with β-arrestin based on the arrestin recruitment experiments (FIGS. 8A-8C). In some embodiments, further details of the mechanism are examined and require detailed structural and computational biology analyses.

### 2.2. Biological Effects of PAM and NAM and Clinical Significance.

It is well-appreciated that the therapeutic efficacy of β-agonists involves complex interplay of interaction between the β₂AR and G proteins, kinases, and β-arrestins. GRKs and β-arrestins are key regulatory molecules involved in agonist-specific desensitization of numerous GPCRs, limiting the capacity of receptor to maintain signaling, and the associated functional consequences. β-agonists are used as both prophylaxis and treatment for asthma, based on their ability to reverse bronchoconstriction caused by ASM contraction. Yet chronic β-agonist use by asthmatics is associated with a loss of drug efficacy, including a loss of bronchoprotective effect, and worsening of asthma control. Studies using ASM cells and murine models of asthma, strongly implicate GRK- and β-arrestin-mediated β₂AR desensitization in the loss of bronchoprotection by β-agonists, as molecular and genetic strategies targeting these regulatory molecules augment and sustain β₂AR-stimulated cAMP and PKA signaling, while increasing β₂AR-agonist-mediated ASM relaxation and bronchorelaxation. In addition, numerous recent studies have also implicated β-arrestin2 in promoting β₂AR-dependent development of the asthma phenotype in murine models of asthma presumably via non-canonical β₂AR signaling that occurs via the formation of a β-arrestin2 scaffold. Thus, identification of agents that help overcome the constraints imposed by GRKs/arrestins represent exciting therapeutic candidates.

In embodiments, augmenting Gs-cAMP signaling by β₂AR provides therapeutic benefit. In this regard, to a limited extent, phosphodiesterase inhibitors can augment canonical β₂AR-cAMP signaling. Yet their use in asthma appears to have limited efficacy and is complicated by significant off-target effects. PAMs provide an alternative means of augmenting β₂AR-cAMP signaling. While one other β₂AR PAM has been reported to date, its characterization has been largely limited to signaling studies of HEK293 cells. In this Example, PAMs that increase cAMP signaling transduced by ISO in human ASM cells were identified. Considering the endogenous level of β₂AR expression in human ASM the modulatory effects of AMs are different in ASM cells compared to that observed in the HEK293 cells. Consistent with previous studies establishing the functional consequences of increasing ASM β₂AR-cAMP signaling, compound 1001 significantly increased the ISO-induced relaxation of contracted human ASM cells and human and murine airways. This Example provides a proof-of-concept that ligands binding to a unique allosteric site on the β₂AR can modulate cAMP signaling in ASM cells, which is useful given the prominent role of canonical β₂AR-Gs-cAMP in regulating cellular functions in multiple organ systems.

Coinciding with the promotion of Gs-cAMP signaling, interaction of agonist-occupied β₂AR with GRKs results in phosphorylation of the receptor and recruitment of arrestins followed by desensitization of β₂AR and activation of non-canonical signaling. Given activation of arrestin-mediated signaling by β-agonists is dose- and time-dependent, the ability of PAM to achieve a Gs-mediated relaxation at a lower β-agonist concentration is desirable. For at least these reasons, molecules that bias β₂AR signaling toward Gs without affecting β-arrestin recruitment can be useful to limit/avoid problems demonstrated with use of balanced β-agonists.

In summary, this Example identifies a novel allosteric binding site on the β₂AR activation of which selectively enhances canonical Gs-cAMP signaling in both cell lines transduced with β₂AR and physiologically relevant cells. Importantly, such signaling modulation translates into increased β₂AR function in ASM cells and tissues, rendering compound 1001 and similar PAMs promising therapeutic tools in the management of obstructive lung diseases.

### 3. Materials and Methods

### 3.1. Computational Methods.

### 3.1.1. Generation of Intermediate β₂AR Conformations.

Climber program was used for identification of intermediate conformations between the active and inactive conformations. Initial coordinates for the active and inactive states were taken from crystallographic structures of the β₂AR PDB ID 3SN6 and 5X7D, respectively. Structure 3SN6 includes the Gs protein along with the GPCR, yielding the active conformation, while 5X7D includes the orthosteric antagonist, carazolol, and a negative allosteric modulator, termed Cmpd-15PA, representing an inactive conformation of the receptor. To generate full atomistic models, missing residues 176-178 (ECL2) and 240-264 (ICL3) required modelling in the active conformation, while residues 232-261 (ICL3) were modeled for the inactive conformation. Generation of complete atomic structures for both conformations were performed with Modeller (version 9.11). A total of 100 conformations were generated for each loop region, and the model with lowest DOPE score was selected for the final protein structure.

The modeled complete structures of the active and inactive states were then used together with Climber to generate conformations intermediate to the two states. Climber allows for morphing between two protein conformations by transitioning two structures, here represented by the active and inactive β₂AR models, in a stepwise, non-linear fashion. The morphing is achieved based on the root-mean-square difference (RMSD) of backbone and side chain atom pairs between the two states and updated throughout the morphing. Here, the active conformation was used as the initial conformation and the inactive was chosen as the final conformation. The methodology creates sets of individual atom pairs for each generated conformation between the initial and final states, A (active) and B (inactive), respectively. Atom pairs then define the restrained distance between conformation A and B based on i) alpha carbons where the distance between each pair is greater than 10 Å and ii) side chain atom pairs less than 10 Å. The inter-residue distances of conformation A are then pulled towards the final conformation using harmonic restraints applied to each atom pair. Based on the progress towards the target conformation, as defined by the coordinate RMSD, the force constant to transform the conformation from the initial conformation to the final is either increased or relaxed, such that if the coordinate RMSD between a specific atom pair is increased or unchanged, the force constant is increased, and vice versa. At each step, the morphed conformation is minimized using the ENCAD potential energy function.

### 3.1.2. Molecular Dynamics.

Molecular dynamics (MD) simulations were run on the step 74 and 143 conformations from the Climber morphing process. The selected conformations were placed in an aqueous periodic system comprised of a lipid bilayer of POPC and cholesterol at a 90/10 ratio, together with 0.15 M NaCl using the CHARMM-GUI membrane builder. Standard protein N- and C-termini were added and disulfide bonds between C106-C191 and C190-C184 were maintained. For minimization and equilibration, the standard CHARMM-GUI membrane builder GROMACS protocol was used. This includes 5000 steps of energy minimization using the steepest decent method with constraints applied on all non-hydrogen atoms. This is followed by a short MD relaxation phase of 50 ps initialized with a 1 fs time step. Temperature was maintained at 303.15 K using the Berendsen thermostat. A subsequent 325 ps MD simulation had the pressure maintained at 1 bar using the Berendsen pressure coupling and the time step was increased to 2 fs. All position restraints were gradually turned off during the relaxation phase. Following initial relaxation, production runs were performed with a time step of 2 fs and temperature and pressure were maintained at 303 K and 1 ATM with the Nose-Hoover method and the Parrinello-Rahman barostat, respectively. Relaxation and production runs used the LINCS algorithm to constrain covalently bound hydrogens and the Leap Frog integrator. Long-range electrostatics were treated with the particle mesh Ewald method with a real space cutoff of 12 Å, a cubic B-spline interpolation and maximal grid-spacing set to 1.2 Å. All simulations were performed with GROMACS (version 2018.1) and the CHARMM36 lipid and protein force field parameters. Ligand parameters were derived from CGenFF and water molecules were treated using the TIP3P model. Each of the two Climber generated conformations were run in three repeats for 292 and 192 ns for Step 74 and 143, respectively, with randomized initial velocities generated from the Boltzmann distribution at 303.15 K. The systems were simulated either unbound (apo) and with agonist (BI-167107, BIA) or an antagonist (Carazolol, CAU) bound, generating a total of 18 different simulations. Initial ligand placement was done by RMSD aligning the corresponding active or inactive crystallographic structure to the respective intermediate conformation based on the protein non-hydrogen atoms. Coordinates were saved every 10 ps from the MD simulations for analysis.

### 3.1.3. Analysis of Active-Like Conformations.

To define possible intermediate conformations from the MD simulations, the inter-helical distance between TM3 and TM6 was monitored. The distance was between the Cα atoms of residues R131 and E265, known as the ionic lock. Structures from the MD simulations at an interval of 1 ns using GROMACS were extracted with all the snapshots from the 9 simulations associated with the step 74 and 143 conformations combined into two sets. Clustering was then performed using the measure cluster command available in the program Visual Molecule Dynamics (VMD), based on protein backbone RMSD using a distance cutoff of 1 Å. Highly flexible residues belonging to ICL3 were not part of the clustering (residues 235 to 264) as the loop displayed large conformational diversity throughout the MD simulations.

### 3.1.4. SILCS FragMap Generation.

SILCS simulations for each conformation involved 10 individual Grand Canonical Monte Carlo (GCMC)/MD simulations each of 100 ns of MD yielding 1 ps of MD for each conformation, as detailed below. Empirical force field parameters for the lipid and protein were from the CHARMM36 and CHARMM36m parameter sets and those for the SILCS solutes were derived from CGenFF. Water molecules were treated with the TIP3P water model. The conformations were placed in lipid bilayers using the Membrane-builder tool available at CHARMM-GUI, as described above. GROMACS was used to insert solutes in the Step74A and Step74B conformations, respectively, corresponding to an approximate concentration of 0.25 M in the presence of 55 M water. Solutes included in the simulations were benzene, propane, methanol, formamide, acetaldehyde, imidazole, acetate, and methylammonium. Ten individual simulation systems were generated for each conformation Step74A and Step74B with randomized initial solute positions. To enhance sampling of the side chain conformations, all protein residues with solvent accessible surface area (SASA) larger than 0.5 Å² had their side chain chi dihedral rotated to increments of 36° in the 10 individual SILCS systems for each conformation. SASA were calculated in GROMACS. Each system was minimized for 5000 steps using the steepest decent (SD) algorithm, followed by 100 ps MD using the velocity rescaling thermostat with initial velocities randomized. The Berendsen barostat was used for initial relaxation of the system volume followed by 0.5 ns of MD using the Leap-Frog integrator. To limit large conformational changes, weak harmonic restraints were applied to the Cα atoms using a force constant of 0.12 kcal/mol/A², which is applied throughout the entire simulation.

The simulations were run for 125 cycles of GCMC/MD. The initial 25 cycles comprise only the GCMC steps where solute and water molecules closest to the protein are redistributed. The production GCMC/MD cycles are comprised of 200,000 GCMC steps followed by 1 ns of MD simulation. During the GCMC portion of each cycle, water and SILCS solutes were swapped between the gas-phase reservoirs and the GCMC portion of the simulation system as driven by the excess chemical potential. To ensure comprehensive sampling of the solutes, the excess chemical potential was varied at every third cycle to maintain average solute concentrations close to the target concentration of 0.25 M. Atomic coordinates at the end of each GCMC round are used as the starting configuration for the subsequent MD simulation. For the MD simulations, the Nose-Hoover method and the Parrinello-Rahman barostat were used to maintain temperature and pressure of 303 K and 1 bar, respectively. Atomic coordinates were saved every 20 ps. The GCMC/MD protocol was performed the SILCS software suite (SilcsBio, LLC). MD simulations were conducted using the GROMACS program.

3D fragment probability distributions for selected solute atoms were generated to create functional group specific and generic FragMap types, as described previously. Atom coordinates were partitioned into lxlx1 Å volume grid elements encompassing the entire simulation system and calculating selected solute atoms local occupancy over the course of the trajectories. The calculated probability distributions were then normalized with respect to the concentration of the solutes in the simulation system based on the relative number of solute molecules to water molecules with water assigned a concentration of 55 M. Boltzmann transformation was then used to convert the normalized probability distributions to grid free energy (GFE) FragMaps.

### 3.1.5. Allosteric Site Identification.

SILCS-Hotspots, used for identifying putative allosteric sites, applied a library of 101 mono and bicyclic ring-containing fragments found in drug-like molecules. The fragments were docked to the entire 3D structures of the proteins utilizing an exhaustive SILCS-Monte Carlo (MC) approach, as previously described. This involved dividing the protein into separate sub-volumes with each fragment in the library docked 1000 times into each sub-volume based on initial random placement of the ring systems using the exhaustive SILCS-MC protocol. SILCS-MC involves relaxation of molecular orientation and conformation in the field of FragMaps based on the ligand grid free energy (LGFE) score along with the intramolecular energy based on CGenFF. The LGFE is calculated based on the overlap of selected atoms in each compound with the corresponding GFE FragMap, assigning each atom that GFE value, and then summing over all the classified atoms in the molecule. The SLLCS-MC docking protocol involves 10,000 MC steps of up to 1 Å, 180°, and 180° molecule translations, rotations, and dihedral rotations followed by 40,000 steps of simulated annealing from 300 to 0 K with 0.2 Å, 90, and 9° molecule translations, rotations, and dihedral rotations from which minimized orientations and LGFE for the fragments were obtained. Fragment binding sites, termed Hotspots, were then selected by clustering the individual fragments, based on center-of-mass, to identify local binding sites of each fragment type. This was followed by a second clustering over all the fragment types to determine the types of fragments occupying each Hotspot. The Hotspots are then ranked based on the average LGFE for all fragments that occupy to the specific hotspot.

### 3.1.6. In Silico Database Screening.

The SILCS-Pharmacophore (SILCS-Pharm) protocol was used to prepare pharmacophore features that were used for the initial step of the virtual screening (VS) campaign. SILCS-Pharm used the information of the SILCS FragMaps to generate pharmacophore features within a chosen radius of selected Hotspots. For each receptor conformation SILCS-Pharm generated 10 to 15 pharmacophore features. These are then manually analyzed to select individual pharmacophore hypotheses that contain 4 or 5 features, including at least 2 apolar features. For each protein structure four hypotheses were generated yielding a total of 8 pharmacophore hypotheses for VS. The Pharmer software was used to perform the VS on an in-house curated drug-like molecule database. The database contains a maximum of 100 conformations for each ligand along with all protonation states from the vendors of Chembridge and Maybridge. The full database contains ˜721,000 unique molecules and ˜1.7 million molecules including protomers.

From the pharmacophore screen the top ˜10,000 compounds were selected for each pharmacophore hypothesis based on low RMSD between the features and corresponding compound functional groups. These lists were then pooled, yielding a total of 80,000 compounds, not including repeated compounds, and subjected to rescoring using SILCS-MC pose refinement. SILCS-MC pose refinement allows for each molecule to further relax in the field of SILCS FragMaps. The molecular conformation is optimized in the field of FragMaps to optimize the LGFE score. In this refinement step, the SILCS-MC pose-refinement protocol corresponds to a local conformational sampling, to prevent the ligand orientations to deviate significantly from the initial orientations obtained from the pharmacophore screens. Duplicate compounds were removed with the best LGFE score maintained for final ranking, all the compounds were then sorted based on LGFE, and a final list of the top 1000 scored compounds were retained. Due to the pharmacophore-based screen there is a potential for a large number of hits with very closely related chemical scaffolds. To filter these similarity clustering was performed using the BIT MACCS fingerprint from which clusters of compounds with similar chemical scaffolds were obtained using the program MOE (Chemical Computing Group). The compounds in each cluster were then sorted based on the LGFE scores with the final 50 compounds for experimental validation selected based on 4-dimensional bioavailability (4DBA) metric based on physicochemical characteristics that maximize the potential for better bioavailability.

### 3.2. Preparation of β₂AR Modulators.

Compounds were purchased from Chembridge (San Diego, CA). The compounds were procured at 5-25 mmol quantity and dissolved in 10% DMSO to prepare 10 mM stock solutions.

### 3.3. Cell Culture.

### 3.3.1. HEK293 Cells.

HEK293 cell lines were obtained from the American Type Culture Collection (ATCC) and maintained in Dulbecco's modified Eagle's medium (DMEM) as described previously.

### 3.3.2. Human Airway Smooth Muscle (ASM) Cells.

Human ASM cells were isolated from deidentified donor lungs and cultured using F-12 media supplemented with 10% FBS, penicillin and streptomycin, HEPES buffer, CaCl₂), L-Glutamine (Gibco, Waltham, MA), and NaOH between 2-6 passages as described previously. Donor lungs were obtained from National Disease Research Institute and use of cells and tissues from deidentified donors have been judged to be Not Human Subjects research by TJU IRB.

3.4. Transfection of HEK293 Cells (cDNA Constructs and Transfections).

The human β₂AR cDNA containing 3-HA tag at N-terminus subcloned into a pcDNA3.1 was obtained from www.cdna.org. Binding site residues PHE(F)282, TYR(Y)219, and ARG(R)131 were mutated to different amino acids using the QuikChange 11 XL Site-Directed Mutagenesis Kit (Agilent, Santa Clara, CA) per manufacturer's protocol. Mutations were confirmed by DNA sequencing. HEK293 cells were transfected with cDNA expressing (wild type or mutant; see below) human β₂AR using Lipofectamine 3000 (Invitrogen, Waltham, MA) and used for studies within 36-48 h, or were maintained in media containing G418 at 500 μg/ml concentration. Expression of β₂AR was confirmed by western blotting and confocal imaging using an anti-HA antibody.

3.5. cAMP Assay.

Cells were grown to full confluency in 24-well plates, serum-starved for 24 h, and then incubated with 1 mM IBMX for 10 min. Cells were then incubated with ISO (1 nM to 10 μM) or β₂AR modulators alone or concomitantly for 10 min and cAMP levels were measured by ELISA (Life Technologies, Waltham, MA) as described previously.

### 3.6. Arrestin Recruitment to the β₂AR Assay: Bioluminescence Resonance Energy Transfer (BRET).

β-agonist-induced recruitment of β-arrestin to the β₂AR was assessed by BRET assay as described previously. HEK293 cells were transfected with pcDNA-β-arrestin2-GFP10 and pcDNA3-β₂AR-RlucII and after 48 h the cells were treated with increasing concentration of ISO and vehicle or 100 nM PAMs or NAMs followed by the addition of Coelenterazine 400a for 20 mins. Change in BRET was measured using a Tecan Infinite F500 microplate reader. BRET ratios were calculated by dividing the intensity of light emitted by the GFP10 acceptor by the total light emitted by the RLucII donor.

### 3.7. Radioligand Binding Assay.

Competitive radioligand binding assay was performed as previously described. Cells expressing β₂AR were lysed and cell membranes were incubated in 3 nM [³H]-dihydroalprenolol ([³H]DHA) with increasing concentration of ISO±AMs. Bound radioactivity was measured with a TriCarb 4910 TR liquid scintillation analyzer (PerkinElmer, Waltham, MA) and expressed as % of maximum [³H]DHA specific binding. Non-specific binding was assessed with 10 μM alprenolol. Binding competition curves were generated using non-linear regression curve fitting function log(inhibitor) vs. response (three parameter) in GraphPad Prism and normalized to percentage of maximal [³H]DHA specific binding.

### 3.8. Immunoassay.

Cells were lysed in RIPA buffer (Cell Signaling Technology, Denvers, MA) containing protease and phosphatase inhibitors (Bimake, Houston, TX) and proteins were separated using SDS-PAGE, transferred onto nitrocellulose membranes and probed with primary antibodies phospho-ERK1/2 and β-actin. Secondary antibody (Li-Cor) was used to quantify target protein using Odyssey scanner (Li-Cor, Lincoln, NE) and analyzed using ImageStudio software (Li-Cor, Lincoln, NE) as described previously.

### 3.9. Receptor Internalization.

ISO induced receptor internalization was assessed via cell surface ELISA as previously described. Briefly, HEK-293 cells expressing β₂AR were plated on 24-well plates. Cells were treated with 100 nM ISO with vehicle or 100 nM AMs for 15 minutes. Post treatment, cells were washed and fixed with 10% formalin-buffered saline for 10 minutes on ice, washed twice, and further processed using horseradish peroxidase-conjugated chicken polyclonal anti-HA antibody (Abcam). Cells were washed and incubated with 3,3′,5,5′-tetramethylbenzidine substrate and transferred to a 96-well plate containing sulfuric acid to stop the colorimetric reaction. FlexStation3 was used to measure absorbance at 450 nm. All values were normalized to basal value (no ISO/AMs) of wild-type receptor.

### 3.10. ASM Cell Contraction Using Collagen Gel.

100 μl of ASM cell suspension (5×10⁵ cells/ml) containing type-I rat tail collagen (1.5 mg/ml) in F-12 medium was seeded onto a 96-well plate. After 24 h, collagen gels were loosened from the plate. The contraction was initiated by adding histamine (10 μM), with or without pretreatment with increasing concentration of ISO, with vehicle or AMs for 10 min prior to histamine. Images were obtained using an EVOS live tissue microscope before and at 10 min intervals for 40 min after the addition of histamine. Gel area was obtained for each of the mages using NIH ImageJ software. Data were expressed as percentage gel contraction and calculated using the following equation. [(T0 gel area−T10 gel area)/T0 gel area×100%].

### 3.11. Ex Vivo Airway Functional Studies Using Murine Precision-Cut Lung Slices.

FVB mice expressing human β₂AR in smooth muscle were used in this study. The protocol for animal experiments is approved by the Institutional Animal Care and Use Committee. Murine lungs were perfused with 4% low melting agarose, allowed to solidify at 4° C. for 1 h. For human airways, a small lobe was inflated with 2% low-melting-point agarose and cores of 8 mm in diameter were made. Both human and murine lungs were then sliced (˜250 μm thick) using an oscillating tissue slicer (OTS-5000, FHC Inc). Lung slices cultured for 48 h were treated with methacholine (MCh, 1 μM), and images of the airways were obtained before and 10 min after MCh stimulation using EVOS FL Auto (Life technologies, Waltham, MA) microscope. The lung slices were treated with ISO (1 nM-10 μM) with vehicle or AMs and images of the airways were captured at 10 min intervals for a total of 50 min. Images of airways were used to calculate the area of airways using Image J software (NIH). Data from lung slice experiments were plotted as a percentage of methacholine-induced decrease in airway area compared to baseline area.

### 3.12. Statistical Analysis.

All data are presented as mean±SEM values from ‘n’ number of lines derived from different donors in the case of human ASM cells and human and murine tissues or technical replicates in the case of HEK293 cells. Densitometry data from western blot analyses are normalized using band intensities in vehicle-treated cells. cAMP concentrations were determined by extrapolation from a standard curve. One/Two-way ANOVA with Bonferroni post hoc analysis or Student's t-test was used to determine statistical differences among treatment groups using GraphPad Prism VI software (La Jolla, CA) as described in drawing descriptions. A p≤0.05 was considered sufficient to reject the null hypothesis.

### Example 2: In Vivo Studies of Compound 1001

This Example describes studies examining the activity of compound 1001 in vivo.

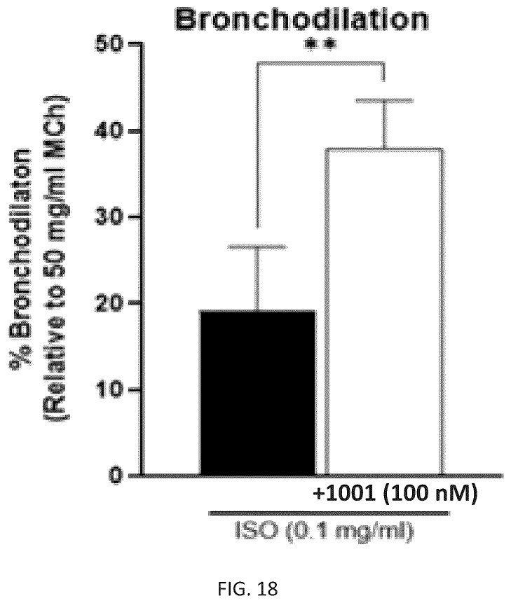

Compound 1001 augments isoproterenol (ISO)-induced bronchodilation in mice. Change in methacholine (MCh)-induced bronchoconstriction (lung resistance) upon aerosol treatment of mice with MCh followed by aerosol treatment of mice with ISO with vehicle or compound 1001 using flexiVent (Scireq) method was assessed. While not wishing to be bound by any particular theory, the data suggested augmentation of ISO-induced bronchodilation by compound 1001 (compared to vehicle) in mice (FIG. 18).

In vivo lung function measurements. Lung mechanics are studied using flexiVent (Scireq) method. Briefly, anesthetized, intubated mice are ventilated with a tidal volume of 250 μl at 150 breaths/min. Mouse airways are exposed to increasing doses of methacholine (MCh; 1.56-50 mg/ml) using an in-line ultrasonic nebulizer. Following MCh-induced bronchoconstriction, mice are aerosolized with increasing concentrations of β-agonists (0.1-20 mg/ml)+AMs (100 nM). Airway resistance (Rn), tissue damping (G), and elastance (H) data (normalized to body weight) at individual doses of agonists are determined using the flexiWare software.

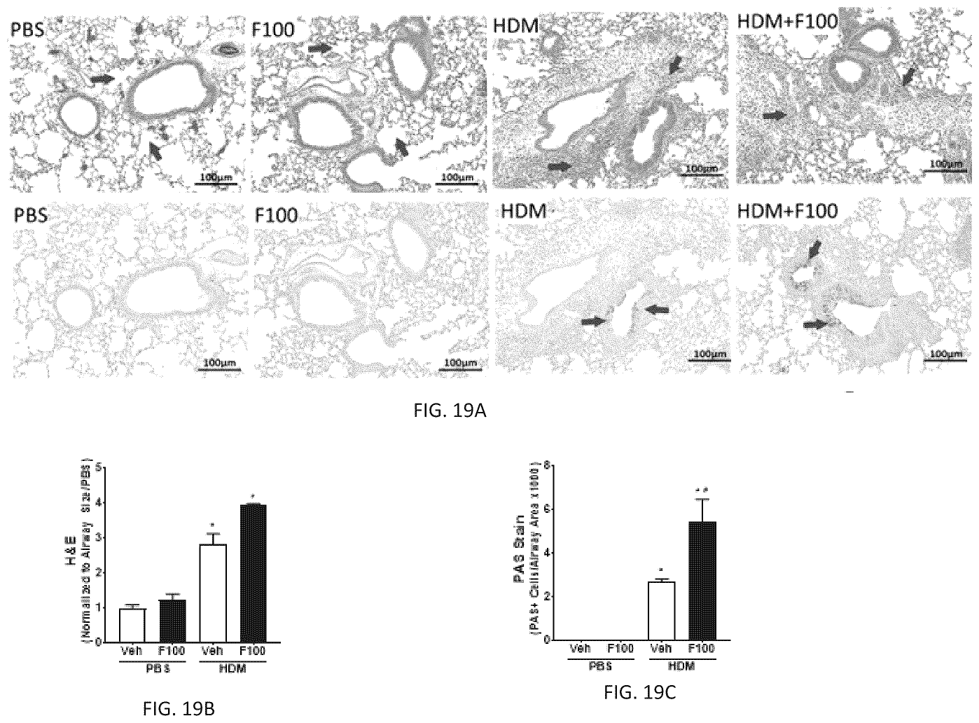

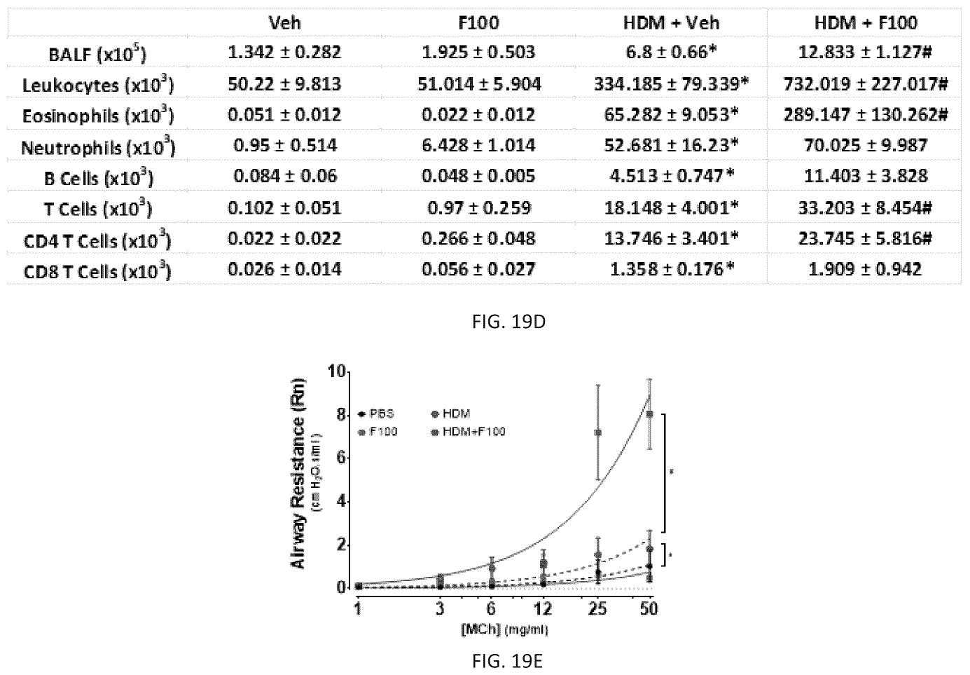

Murine model of allergic asthma. Mice were challenged with House dust mite (HDM) (25 μg/mouse in 35 μl) intranasally (i.n.) for 5 days a week for a total of 3 weeks and phenotype was assessed. A 3 week HDM challenge resulted in significant airway inflammation (bronchoalveolar lavage (BAL) cellularity and cytokines), goblet cell metaplasia (PAS), and increased bronchoconstrictor responsiveness to inhaled MCh treatment consistent with airway hyperresponsiveness (AHR) (FIGS. 19A-E). Further, i.n. treatment of animals with formoterol (100 μg/kg) further enhanced the markers of HDM-induced airway inflammation, mucus cell metaplasia, and AHR (FIGS. 19A-E). Features of airway remodeling including thickening of airway wall (H&E), lung extracellular matrix (ECM) accumulation (trichrome) and hyperproliferation of lung cells (Ki-67), and secretion of cytokines eotaxin, IL-5, IL-13 IL-6, KC, IL-10, MCP-1, IL-17, and G-CSF in BAL were also enhanced by formoterol. An established HDM model is used to test the effect of β-agonists±Positive Allosteric Modulators (PAMs) on the development of asthma phenotype. In another non-limiting example, an ovalbumin model is employed.

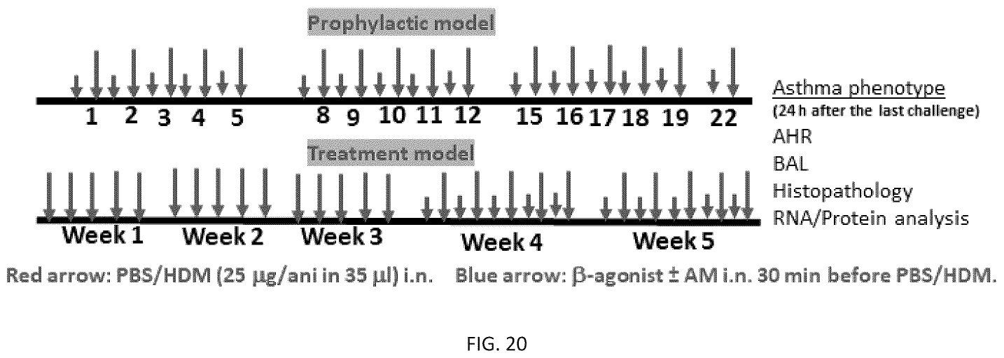

Allergen challenge and treatment protocol (prophylaxis model): Mice are challenged (i.n.) with 1) phosphate-buffered saline (PBS) or 2) HDM (FIG. 20). PBS-challenged animals are pretreated i.n. as follows: 3-5) formoterol (10, 50 & 100 μg/kg)+vehicle; 6-8) compound 1001 (5, 10, & 20 μg/kg), and 9) formoterol (100 μg/kg)+compound 1001 (10 mg/kg) 10 min before PBS treatment. HDM-challenged animals are pretreated i.n. as follows: 10-12) formoterol (10, 50 & 100 μg/kg)+vehicle; 13-15) compound 1001 (5, 10, & 20 μg/kg), and 16) formoterol (100 μg/kg)+compound 1001 (10 μg/kg) 10 min before HDM challenge. Dose are chosen based on EC₅₀/Eₘₐₓ values obtained from in vitro studies, pilot data, and previous in vivo studies. A pilot study is conducted to determine a dose of the formoterol, compound 1001, and combination therapy. The dose and treatment regimen are refined empirically more data from both in vitro and in vivo studies are generated. In a non-limiting example, only one PAM (compound 1001; characterized in in vitro studies) and one β-agonist (formoterol, FIG. 20) are used in the study. In another non-limiting example, two or more PAMs are used in the study.

### REFERENCES

1. D. A. Deshpande, R. B. Penn, Targeting G protein-coupled receptor signaling in asthma. Cell Signal 18, 2105-2120 (2006).
2. S. R. Salpeter, An update on the safety of long-acting beta-agonists in asthma patients using inhaled corticosteroids. Expert Opin Drug Saf 9, 407-419 (2010).
3. S. R. Salpeter, N. S. Buckley, T. M. Ormiston, E. E. Salpeter, Meta-analysis: effect of long-acting beta-agonists on severe asthma exacerbations and asthma-related deaths. Ann Intern Med 144, 904-912 (2006).
4. W. O. Spitzer et al., The use of beta-agonists and the risk of death and near death from asthma. N Engl J Med 326, 501-506 (1992).
5. D. R. Taylor, The beta-agonist saga and its clinical relevance: on and on it goes. Am J Respir Crit Care Med 179, 976-978 (2009).
6. H. S. Nelson et al., The Salmeterol Multicenter Asthma Research Trial: a comparison of usual pharmacotherapy for asthma or usual pharmacotherapy plus salmeterol. Chest 129, 15-26 (2006).
7. J. K. Walker, R. B. Penn, N. A. Hanania, B. F. Dickey, R. A. Bond, New perspectives regarding beta(2)-adrenoceptor ligands in the treatment of asthma. Br J Pharmacol 163, 18-28 (2011).
8. R. B. Penn, R. A. Bond, J. K. Walker, GPCRs and arrestins in airways: implications for asthma. Handh Exp Pharmacol 219, 387-403 (2014).
9. D. A. Deshpande, B. S. Theriot, R. B. Penn, J. K. Walker, Beta-arrestins specifically constrain beta2-adrenergic receptor signaling and function in airway smooth muscle. FASEB J 22, 2134-2141 (2008).
10. D. A. Deshpande et al., Exploiting functional domains of GRK2/3 to alter the competitive balance of pro- and anticontractile signaling in airway smooth muscle. FASEB J 28, 956-965 (2014).
11. L. P. Nguyen et al., beta2-Adrenoceptor signaling in airway epithelial cells promotes eosinophilic inflammation, mucous metaplasia, and airway contractility. Proc Natl Acad Sci USA 114, E9163-E9171 (2017).
12. T. Pera et al., Specificity of arrestin subtypes in regulating airway smooth muscle G protein-coupled receptor signaling and function. FASEB J 29, 4227-4235 (2015).
13. N. Al-Sawalha et al., Epinephrine Activation of the beta2-Adrenoceptor Is Required for IL-13-Induced Mucin Production in Human Bronchial Epithelial Cells. PLoS One 10, e0132559 (2015).
14. N. A. Hanania et al., Response to salbutamol in patients with mild asthma treated with nadolol. Eur Respir J 36, 963-965 (2010).
15. R. Joshi et al., Effects of beta-blockers on house dust mite-driven murine models pre- and post-development of an asthma phenotype. Pulm Pharmacol Ther 46, 30-40 (2017).
16. J. M. Knight et al., Long-Acting Beta Agonists Enhance Allergic Airway Disease. PLoS One 10, e0142212 (2015).
17. V. J. Thanawala et al., beta2-Adrenoceptor agonists are required for development of the asthma phenotype in a murine model. Am J Respir Cell Mol Biol 48, 220-229 (2013).
18. V. J. Thanawala et al., beta-Blockers have differential effects on the murine asthma phenotype. Br J Pharmacol 172, 4833-4846 (2015).
19. A. Bock, M. Bermudez, Allosteric coupling and biased agonism in G protein-coupled receptors. FEBS. 1288, 2513-2528 (2021).
20. P. J. Conn, A. Christopoulos, C. W. Lindsley, Allosteric modulators of GPCRs: a novel approach for the treatment of CNS disorders. Nat Rev Drug Discov 8, 41-54 (2009).
21. X. Liu et al., Mechanism of intracellular allosteric beta2AR antagonist revealed by X-ray crystal structure. Nature 548, 480-484 (2017).
22. X. Liu et al., An allosteric modulator binds to a conformational hub in the beta2 adrenergic receptor. Nat Chem Biol 16, 749-755 (2020).
23. X. Liu et al., Mechanism of beta2AR regulation by an intracellular positive allosteric modulator. Science 364, 1283-1287 (2019).
24. S. Ahn et al., Allosteric “beta-blocker” isolated from a DNA-encoded small molecule library. Proc Natl Acad Sci USA 114, 1708-1713 (2017).
25. B. Pani et al., Unique Positive Cooperativity Between the beta-Arrestin-Biased beta-Blocker Carvedilol and a Small Molecule Positive Allosteric Modulator of the beta2-Adrenergic Receptor. Mol Pharmacol 100, 513-525 (2021).
26. J. Wang et al., beta-Arrestin-Biased Allosteric Modulator Potentiates Carvedilol-Stimulated beta Adrenergic Receptor Cardioprotection. Mol Pharmacol 100, 568-579 (2021).
27. O. Guvench, A. D. MacKerell Jr, Computational Fragment-Based Binding Site Identification by Ligand Competitive Saturation. PLoS Comp. Biol. 5, e1000435 (2009).
28. E. P. Raman, W. Yu, S. K. Lakkaraju, A. D. Mackerell, Jr., Inclusion of Multiple Fragment Types in the Site Identification by Ligand Competitive Saturation (SILCS) Approach. Journal of chemical information and modeling 53, 3384-3398 (2013).
29. D. R. Weiss, M. Levitt, Can morphing methods predict intermediate structures? J Mol Biol 385, 665-674 (2009).
30. A. D. MacKerell, Jr., S. Jo, S. K. Lakkaraju, C. Lind, W. Yu, Identification and characterization of fragment binding sites for allosteric ligand design using the site identification by ligand competitive saturation hotspots approach (SILCS-Hotspots). Biochim Biophys Acta Gen Subj 1864, 129519 (2020).
31. G. A. Heinzl et al., Iminoguanidines as Allosteric Inhibitors of the Iron-Regulated Heme Oxygenase (HemO) of Pseudomonas aeruginosa. J Med Chem 59, 6929-6942 (2016).
32. A. Gomes et al., Insights into Glucose-6-phosphate Allosteric Activation of beta-Glucosidase A. Journal of chemical information and modeling 61, 1931-1941 (2021).
33. W. Yu, S. K. Lakkaraju, E. P. Raman, L. Fang, A. D. MacKerell, Jr., Pharmacophore Modeling Using Site-Identification by Ligand Competitive Saturation (SILCS) with Multiple Probe Molecules. Journal of chemical information and modeling 55, 407-420 (2015).
34. E. P. Raman, W. Yu, O. Guvench, A. D. Mackerell, Reproducing crystal binding modes of ligand functional groups using Site-Identification by Ligand Competitive Saturation (SILCS) simulations. Journal of chemical information and modeling 51, 877-896 (2011).
35. S. K. Lakkaraju et al., Mapping Functional Group Free Energy Patterns at Protein Occluded Sites: Nuclear Receptors and G-Protein Coupled Receptors. Journal of chemical information and modeling 55, 700-708 (2015).
36. S. K. Lakkaraju, E. P. Raman, W. Yu, A. D. MacKerell, Jr., Sampling of Organic Solutes in Aqueous and Heterogeneous Environments using Oscillating pex Grand Canonical-like Monte Carlo-Molecular Dynamics Simulations. Journal of Chemical Theory and Computing 10, 2281-2290 (2014).
37. D. R. Koes, C. J. Camacho, Pharmer: Efficient and Exact Pharmacophore Search. Journal of chemical information and modeling 51, 1307-1314 (2011).
38. T. Oashi, A. L. Ringer, E. P. Raman, A. D. Mackerell, Automated selection of compounds with physicochemical properties to maximize bioavailability and druglikeness. Journal of chemical information and modeling 51, 148-158 (2011).
39. S. J. Horvat et al., A-kinase anchoring proteins regulate compartmentalized cAMP signaling in airway smooth muscle. FASEB J 26, 3670-3679 (2012).
40. J. Quoyer et al., Pepducin targeting the C-X-C chemokine receptor type 4 acts as a biased agonist favoring activation of the inhibitory G protein. Proc Natl Acad Sci USA 110, E5088-5097 (2013).
41. S. Naik et al., Regulation of cysteinyl leukotriene type 1 receptor internalization and signaling. J Biol Chem 280, 8722-8732 (2005).
42. T. G. Zaremba, P. H. Fishman, Desensitization of catecholamine-stimulated adenylate cyclase and down-regulation of beta-adrenergic receptors in rat glioma C6 cells. Role of cyclic AMP and protein synthesis. Mol Pharmacol 26, 206-213 (1984).
43. S. J. Morgan et al., beta-Agonist-mediated relaxation of airway smooth muscle is protein kinase A-dependent. J Biol Chem 289, 23065-23074 (2014).
44. H. Goel, A. Hazel, W. Yu, S. Jo, A. D. MacKerell, Jr., Application of Site-Identification by Ligand Competitive Saturation in Computer-Aided Drug Design. New J Chem 46, 919-932 (2022).
45. R. C. Bernardi, M. C. R. Melo, K. Schulten, Enhanced sampling techniques in molecular dynamics simulations of biological systems. Biochim Biophys Acta 1850, 872-877 (2015).
46. A. M. Schonegge et al., Evolutionary action and structural basis of the allosteric switch controlling beta2AR functional selectivity. Nat Commun 8, 2169 (2017).
47. S. G. Rasmussen et al., Crystal structure of the beta2 adrenergic receptor-Gs protein complex. Nature 477, 549-555 (2011).
48. S. Chen et al., Mutation of a single TMVI residue, Phe(282), in the beta(2)-adrenergic receptor results in structurally distinct activated receptor conformations. Biochemistry 41, 6045-6053 (2002).
49. R. U. Malik et al., Detection of G protein-selective G protein-coupled receptor (GPCR) conformations in live cells. J Biol Chem 288, 17167-17178 (2013).
50. M. Choi et al., G protein-coupled receptor kinases (GRKs) orchestrate biased agonism at the beta2-adrenergic receptor. Sci Signal 11 (2018).
51. R. J. Lefkowitz, K. L. Pierce, L. M. Luttrell, Dancing with different partners. protein kinase a phosphorylation of seven membrane-spanning receptors regulates their G protein-coupling specificity. Mol Pharmacol 62, 971-974 (2002).
52. S. R. Salpeter, N. S. Buckley, Systematic review of clinical outcomes in chronic obstructive pulmonary disease: beta-agonist use compared with anticholinergics and inhaled corticosteroids. Clin Rev Allergy Immunol 31, 219-230 (2006).
53. S. R. Salpeter, A. J. Wall, N. S. Buckley, Long-acting beta-agonists with and without inhaled corticosteroids and catastrophic asthma events. Am J Med 123, 322-328 e322 (2010).
54. G. S. Forkuo et al., Phosphodiesterase 4 Inhibitors Attenuate the Asthma Phenotype Produced by beta2-Adrenoceptor Agonists in Phenylethanolamine N-Methyltransferase-Knockout Mice. Am J Respir Cell Mol Biol 55, 234-242 (2016).
55. F. J. Nunez et al., Agonist-specific desensitization of PGE2-stimulated cAMP signaling due to upregulated phosphodiesterase expression in human lung fibroblasts. Naunyn Schmiedebergs Arch Pharmacol 393, 843-856 (2020).
56. R. A. Panettieri, R. K. Murray, L R. DePalo, P. A. Yadvish, M. I. Kotlikoff, A human airway smooth muscle cell line that retains physiological responsiveness. Am J Physiol 256, C329-335 (1989).
57. J. V. Michael et al., Cooperativity of E-prostanoid receptor subtypes in regulating signaling and growth inhibition in human airway smooth muscle. FASEB J 33, 4780-4789 (2019).
58. S. K. Yadav et al., Autocrine regulation of airway smooth muscle contraction by diacylglycerol kinase. J Cell Physiol 10.1002/jcp.30528 (2021).
59. F. De Pascali et al., beta2-Adrenoceptor agonist profiling reveals biased signalling phenotypes for the beta2-adrenoceptor with possible implications for the treatment of asthma. Br J Pharmacol 10.1111/bph.15900 (2022).
60. A. P. Nayak et al., Regulation of ovarian cancer G protein-coupled receptor-1 expression and signaling. Am J Physiol Lung Cell Mol Physiol 316, L894-L902 (2019).
61. T. J. Harford et al., Respiratory syncytial virus induces beta2-adrenergic receptor dysfunction in human airway smooth muscle cells. Sci Signal 14 (2021).
62. D. W. McGraw et al., Transgenic overexpression of beta(2)-adrenergic receptors in airway smooth muscle alters myocyte function and ablates bronchial hyperreactivity. J Biol Chem 274, 32241-32247 (1999).
63. A. Bergner, M. J. Sanderson, Airway contractility and smooth muscle Ca(2+) signaling in lung slices from different mouse strains. J Appl Physiol (1985) 95, 1325-1332; discussion 1314 (2003).
64. A. Fiser, A. Sali, ModLoop: automated modeling of loops in protein structures. Bioinformatics 19, 2500-2501 (2003).
65. M. Levitt, M. Hirshberg, R. Sharon, V. Daggett, Potential Energy Function and Parameters for Simulations of the Molecular Dynamics of Proteins and Nucleic Acids in Solution. Comp. Phys. Commun. 91, 215-231 (1995).
66. S. Jo, T. Kim, V. G. Iyer, W. Im, CHARMM-GUI: a web-based graphical user interface for CHARMM. Journal of computational chemistry 29, 1859-1865 (2008).
67. S. Nose, A unified formulation of the constant temperature molecular dynamics method. J. Chem. Phys. 81, 511-519 (1984).
68. W. G. Hoover, Canonical Dynamics—Equilibrium Phase-Space Distributions. Physical Review A 31, 1695-1697 (1985).
69. M. Parrinello, A. Rahman, Polymorphic transitions in single crystals: A new molecular dynamics method. Journal of Applied Physics 52, 7182-7190 (1981).
70. B. Hess, H. Bekker, H. J. C. Berendsen, J. G. E. M. Fraaije, LINCS: A Linear Constraint Solver for molecular simulations. Journal of computational chemistry 18, 1463-1472 (1997).
71. T. A. Darden, D. York, L. G. Pedersen, Particle mesh Ewald: An N log(N) method for Ewald sums in large systems. J. Chem. Phys. 98, 10089-10092 (1993).
72. M. J. Abraham et al., GROMACS: High performance molecular simulations through multi-level parallelism from laptops to supercomputers. SoftwareX 1-2, 19-25 (2015).
73. J. B. Klauda et al., Update of the CHARMM all-atom additive force field for lipids: validation on six lipid types. J Phys Chem B 114, 7830-7843 (2010).
74. R. B. Best et al., Optimization of the additive CHARMM all-atom protein force field targeting improved sampling of the backbone φ, ψ and side-chain χ1 and χ2 dihedral angles. J. Chem. Theory and Comp. 8, 3257-3273 (2012).
75. K. Vanommeslaeghe et al., CHARMM general force field: A force field for drug-like molecules compatible with the CHARMM all-atom additive biological force fields. J Comput Chem 31, 671-690 (2010).
76. W. L. Jorgensen, J. Chandrasekhar, J. D. Madura, R. W. Impey, M. L. Klein, Comparison of Simple Potential Functions for Simulating Liquid Water. J. Chem. Phys. 79, 926-935 (1983).
77. R. O. Dror et al., Activation mechanism of the beta2-adrenergic receptor. Proc. Natl. Acad. Sci. U.S.A 108, 18684-18689 (2011).
78. W. Humphrey, A. Dalke, K. Schulten, VMD: Visual Molecular Dynamics. J. Molec. Graph. 14, 33-38 (1996).
79. J. Huang et al., CHARMM36m: an improved force field for folded and intrinsically disordered proteins. Nature Methods 14, 71-73 (2017).
80. M. Levitt, S. Lifson, Refinement of Protein Conformations Using a Macromolecular Energy Minimization Procedure. Journal of Molecular Biology 46, 269-279 (1969).
81. H. J. C. Berendsen, J. P. M. Postma, W. F. van Gunsteren, A. DiNola, J. R. Haak, Molecular Dynamics with Coupling to an External Bath. J. Chem. Phys. 81, 3684-3690 (1984).
82. H. Goel et al., Rapid and accurate estimation of protein-ligand relative binding affinities using site-identification by ligand competitive saturation. Chemical Science 12, 8844-8858 (2021).
83. R. D. Taylor, M. MacCoss, A. D. Lawson, Rings in drugs. J Med Chem 57, 5845-5859 (2014).
84. V. D. Ustach et al., Optimization and Evaluation of Site-Identification by Ligand Competitive Saturation (SILCS) as a Tool for Target-Based Ligand Optimization. Journal of chemical information and modeling 59, 3018-3035 (2019).
85. N. Parvaiz, F. Ahmad, W. Yu, A. D. MacKerell, Jr., S. S. Azam, Discovery of beta-lactamase CMY-10 inhibitors for combination therapy against multi-drug resistant Enterobacteriaceae. PLoS One 16, e0244967 (2021).
86. A. P. Nayak et al. OGR1-dependent regulation of the allergen-induced asthma phenotype. Am J Physiol Lung Cell Mol Physiol 321, L 1044-L1054, (2021).
87. Sharma, P. et al. Bitter Taste Receptor Agonists Mitigate Features of Allergic Asthma in Mice. Sci Rep 7, 46166, (2017).
88. Shah, S. D. et al. Targeted Inhibition of Select ERK1/2 Functions Mitigates Pathological Features of Asthma in Mice. Am J Respir Cell Mol Biol, (2022).
89. Singh, B. K. et al. Diacylglycerol kinase zeta promotes allergic airway inflammation and airway hyperresponsiveness through distinct mechanisms. Science signaling 12, (2019).

1. A compound of formula (I), or a pharmaceutically acceptable salt, solvate, hydrate, cocrystal, or prodrug thereof: 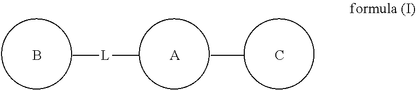
wherein in formula (I):
A is an optionally substituted 6-membered heteroaryl or optionally substituted 6-membered heterocyclyl, provided that the optionally substituted 6-membered heteroaryl or optionally substituted 6-membered heterocyclyl comprises two or more nitrogen atoms;
B is an optionally substituted monocyclic aryl or optionally substituted monocyclic or bicyclic heteroaryl;
C is an optionally substituted heteroaryl or optionally substituted cycloalkyl;
L is a linker comprising one or more of a bond, —NR^{a}—, —S—, —S(O)—, —S(O)₂—, —O—, —CR^{a}₂—, —C(O)O—, —OC(O)—, —C(O)S—, —SC(O)—, —C(O)NR^{a}—, —NR^{a}C(O)—, —C(O)NR^{a}SO₂—, —SO₂NR^{a} C(O)—, —OC(O)O—, —OC(O)S—, —SC(O)O—, —OC(O)NR^{a}—, —NR^{a}C(O)O—, —CR^{a}═N—NR^{a}—, disubstituted alkyl, disubstituted heteroalkyl, disubstituted alkenyl, disubstituted alkynyl, disubstituted cycloalkyl, disubstituted heterocycloalkyl, disubstituted aryl, disubstituted arylalkyl, disubstituted heteroaryl, and/or disubstituted heteroarylalkyl; and
R^{a} is each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl.

2. The compound of claim 1, wherein A is selected from optionally substituted pyrimidine, optionally substituted pyridazine, optionally substituted pyrazine, and optionally substituted piperazine.

3. The compound of claim 2, wherein A is selected from: 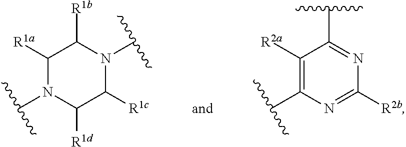
wherein R^{1a}, R^{1b}, R^{1c}, R^{1d}, R^{2a}, and R^{2b} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R³, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(OR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

4. The compound of any one of claims 1-3, wherein B is optionally substituted aryl, optionally substituted pyridyl, or optionally substituted quinoxaline.

5. The compound of any one of claims 1-4, wherein C is optionally substituted 3- to 7-membered cycloalkyl, optionally substituted pyrrole, optionally substituted imidazole, optionally substituted pyrazole, or optionally substituted triazole.

6. The compound of any one of claims 3-5, or a pharmaceutically acceptable salt, solvate, hydrate, cocrystal, or prodrug thereof, wherein the compound has a structure according to formula (1): 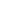
wherein in formula (1):
B is an optionally substituted monocyclic heteroaryl; and
C is an optionally substituted 3- to 7-membered cycloalkyl.

7. The compound of any one of claims 3-5, or a pharmaceutically acceptable salt, solvate, hydrate, cocrystal, or prodrug thereof, wherein the compound has a structure according to formula (2): 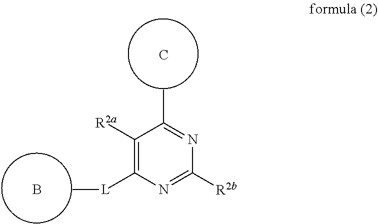
wherein in formula (2):
B is an optionally substituted monocyclic aryl; and
C is an optionally heteroaryl.

8. The compound of any one of claims 1-7, wherein B is selected from: 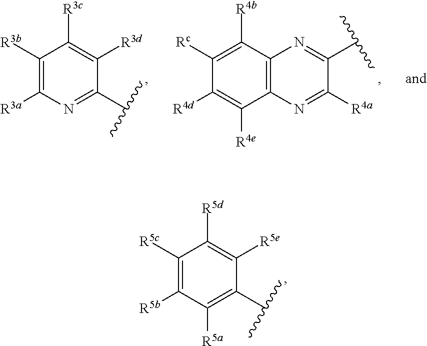
wherein R^{3a}, R^{3b}, R^{3c}, R^{3d}, R^{4a}, R^{4b}, R^{4c}, R^{4d}, R^{4e}, R^{5a}, R^{5b}, R^{5c}, R^{5d}, and R^{5e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)^{t}R^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

9. The compound of claim 8, or a pharmaceutically acceptable salt, solvate, hydrate, cocrystal, or prodrug thereof, wherein the compound has a structure according to formula (10):

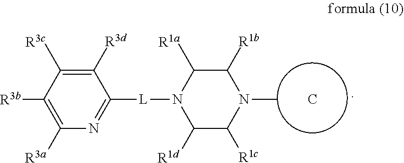

10. The compound of claim 8, or a pharmaceutically acceptable salt, solvate, hydrate, cocrystal, or prodrug thereof, wherein the compound has a structure according to formula (11):

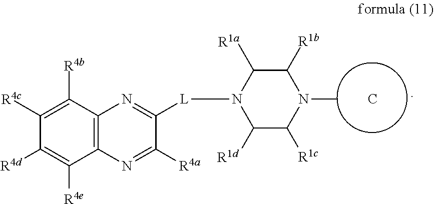

11. The compound of claim 9 or 10, wherein R^{1a}, R^{1b}, and R^{1d} are each H.

12. The compound of any one of claims 9-11, wherein R^{1e} is substituted C₁₋₆ alkyl, optionally substituted ethyl, optionally —(CH₂)₂—OH.

13. The compound of any one of claims 9, 11, or 12, wherein R^{3a}, R^{3b}, R^{3c}, and R^{3d} are each H.

14. The compound of any one of claims 10-12, wherein R^{4b}, R^{4c}, R^{4d}, and R^{4e} are each H.

15. The compound of any one of claims 10-12 or 14, wherein R^{4a} is C₁₋₆ alkyl, optionally —CH₃.

16. The compound of any one of claims 1-6 or 8-15, wherein C is selected from: 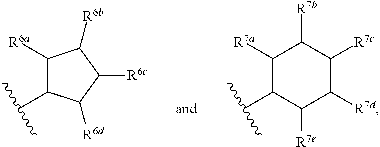
wherein R^{6a}, R^{6b}, R^{6c}, R^{6d}, R^{7a}, R^{7b}, R^{7c}, R^{7d}, and R^{7e} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R^{a})C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R^{b}, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

17. The compound of claim 16, wherein C is: 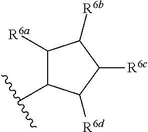
and
R^{6a}, R^{6b}, R^{6c}, and R^{6d} are each H.

18. The compound of claim 16, wherein C is: 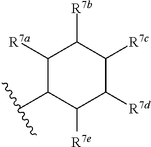
and
R^{7a}, R^{7b}, R^{7c}, R^{7d}, and R^{7e} are each H.

19. The compound of any one of claims 1-18, wherein L is —(CH₂)₁₋₆—, optionally L is —(CH₂)—.

20. The compound of any one of claims 2-5, 7, or 8, or a pharmaceutically acceptable salt, solvate, hydrate, cocrystal, or prodrug thereof, wherein the compound has a structure according to formula (3): 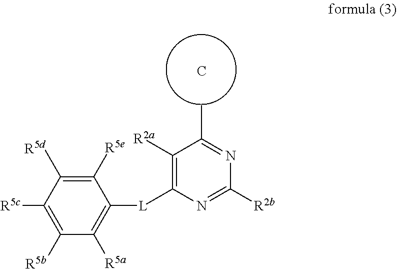
wherein in formula (3):
B is an optionally substituted monocyclic aryl; and
C is an optionally heteroaryl.

21. The compound of claim 20, wherein R^{2a} and R^{2b} are each H.

22. The compound of claim 20 or 21, wherein R^{5b}, R^{5c}, R^{5d}, and R^{5e} are each H.

23. The compound of any one of claims 20-22, wherein R^{5a} is —OR^{a}, optionally —OH.

24. The compound of any one of claims 20-23, wherein C is: 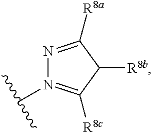
wherein R^{8a}, R^{8b}, and R^{8c} are each independently selected from H, OH, halo, cyano, fluoroalkyl, trifluoromethyl, trifluoromethoxy, nitro, trimethylsilanyl, —OR^{a}, —SR^{a}, —OC(O)R^{a}, —N(R^{a})R^{b}, —C(O)R^{a}, —C(O)OR^{a}, —OC(O)N(R^{a})R^{b}, —C(O)N(R^{a})R^{b}, —N(R^{a})C(O)OR^{a}, —N(R⁸)C(O)R^{a}, —N(R^{a})C(O)N(R^{a})R^{b}, —N(R^{a})C(NR^{a})N(R^{a})R^{b}, —N(R^{a})S(O)ₜR^{a}, —C(O)N(R^{a})S(O)ₜR^{a}, —S(O)ₜOR^{a}, —S(O)ₜN(R^{a})R¹, —S(O)ₜN(R^{a})C(O)R^{b}, or —P(O)(OR^{a})(OR^{b}), optionally substituted alkyl, optionally substituted alkylaryl, optionally substituted alkylheteroaryl, optionally substituted alkenyl, optionally substituted alkynyl, optionally substituted cycloalkyl, optionally substituted aryl, optionally substituted aralkyl, optionally substituted haloalkyl, optionally substituted alkoxy, optionally substituted heterocyclyl, and optionally substituted heteroaryl;
R^{a} and R^{b} are each independently selected from the group consisting of hydrogen, alkyl, fluoroalkyl, cycloalkyl, carbocyclyl, carbocyclylalkyl, aryl, aralkyl, heterocycloalkyl, heterocycloalkylalkyl, heteroaryl, heteroarylalkyl, halogen, —O-alkyl, —O-aryl, cyano, nitro, —OH, —NH₂, —NH-alkyl, and —NH-aryl; and
t is 1 or 2.

25. The compound of claim 24, wherein R^{8a} and R^{8c} are each H.

26. The compound of claim 24 or 25, wherein R⁸ is C₁₋₆ alkyl, optionally —CH₃.

27. The compound of any one of claims 1-26, wherein L is —CR^{a}═N—NR^{a}—, optionally L is —CH═N—NH—.

28. The compound of any one of claims 1-27, wherein the compound is an allosteric activator of the β₂-adrenoceptor.

29. The compound of any one of claims 1-27, wherein the compound is an allosteric inhibitor of the β₂-adrenoceptor.

30. The compound of any one of claims 1-29, wherein the compound is not a compound of any one of formula 1001-1008:

| Compound # | Structure |
|---|---|
| 1001 | 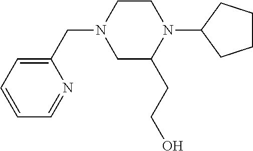 [SMILES: OCCC1CN(Cc2ccccn2)CCN1C1CCCC1] |
| 1002 | 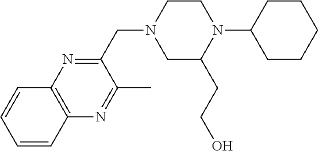 [SMILES: Cc1nc2ccccc2nc1CN1CCN(C2CCCCC2)C(CCO)C1] |
| 1003 | 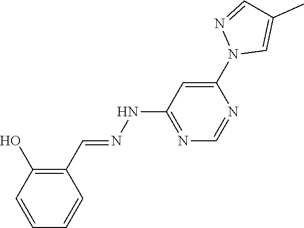 [SMILES: Cc1cnn(-c2cc(N/N=C/c3ccccc3O)ncn2)c1] |
| 1004 | 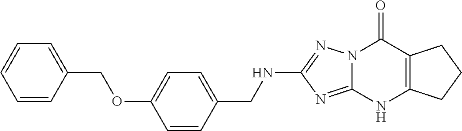 [SMILES: O=c1c2c([nH]c3nc(NCc4ccc(OCc5ccccc5)cc4)nn13)CCC2] |
| 1005 | 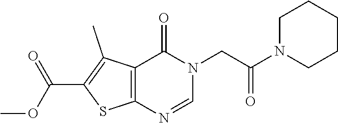 [SMILES: COC(=O)c1sc2ncn(CC(=O)N3CCCCC3)c(=O)c2c1C] |
| 1006 | 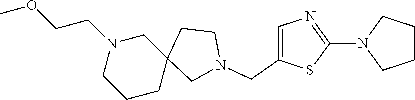 [SMILES: COCCN1CCCC2(CCN(Cc3cnc(N4CCCC4)s3)C2)C1] |
| 1007 | 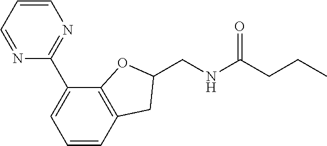 [SMILES: CCCC(=O)NCC1Cc2cccc(-c3ncccn3)c2O1] |
| 1008 | 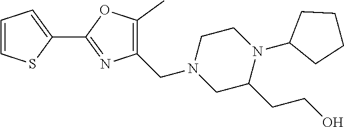 [SMILES: Cc1oc(-c2cccs2)nc1CN1CCN(C2CCCC2)C(CCO)C1] |

31. A pharmaceutical composition comprising a compound of any one of claims 1-30, or a pharmaceutically acceptable salt, solvate, hydrate, cocrystal, or prodrug thereof, and a physiologically compatible carrier medium.

32. A method of treating a disease or disorder alleviated by allosterically modulating β₂-adrenoceptor in a patient in need thereof, the method comprising administering to the patient a therapeutically effective amount of a compound of any one of claims 1-29, or a pharmaceutically acceptable salt, solvate, hydrate, cocrystal, or prodrug thereof.

33. A method of treating a disease or disorder alleviated by allosterically modulating β₂-adrenoceptor in a patient in need thereof, the method comprising administering to the patient a therapeutically effective amount of a pharmaceutical composition of claim 31, or a pharmaceutically acceptable salt, solvate, hydrate, cocrystal, or prodrug thereof.

34. The method of claim 32 or 33, wherein the method further comprises allosterically activating β₂-adrenoceptor.

35. The method of any one of claims 32-34, wherein the disease or disorder is an inflammatory disease.

36. The method of claim 35, wherein the inflammatory disease is selected from the group consisting of chronic obstructive pulmonary disease (COPD), asthma, acute respiratory distress syndrome (ARDS), and acute lung injury (ALI), optionally wherein the inflammatory disease is asthma or COPD.

37. The method of any one of claims 32-34, wherein the disease or disorder is a cardiovascular disease.

38. The method of claim 37, wherein the cardiovascular disease is selected from the group consisting of cardiac arrhythmia, cardiomyopathy, myocardial infarction, heart valve disease, and heart failure, optionally wherein the cardiovascular disease is heart failure.

39. The method of any one of claims 32-38, wherein the compound is of formula 1001, or a pharmaceutically acceptable salt, solvate, hydrate, cocrystal, or prodrug thereof:

| Compound # | Structure |
|---|---|
| 1001 | 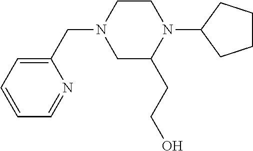 [SMILES: OCCC1CN(Cc2ccccn2)CCN1C1CCCC1] |

40. The method of any one of claims 32-38, wherein the compound is of formula 1002, or a pharmaceutically acceptable salt, solvate, hydrate, cocrystal, or prodrug thereof:

| Compound # | Structure |
|---|---|
| 1002 | 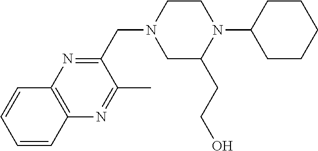 [SMILES: Cc1nc2ccccc2nc1CN1CCN(C2CCCCC2)C(CCO)C1] |

41. The method of any one of claims 32-38, wherein the compound is of formula 1003, or a pharmaceutically acceptable salt, solvate, hydrate, cocrystal, or prodrug thereof:

| Compound # | Structure |
|---|---|
| 1003 | 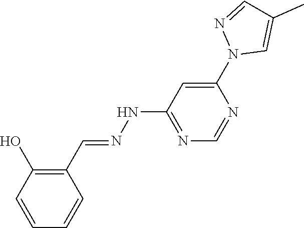 [SMILES: Cc1cnn(-c2cc(N/N=C/c3ccccc3O)ncn2)c1] |

42. The method of any one of claims 32-41, wherein the compound, or pharmaceutically acceptable salt, solvate, hydrate, cocrystal, or prodrug thereof, is administered in a dosage unit form.

43. The method of claim 42, wherein the dosage unit form comprises a physiologically compatible carrier medium.
